(19) United States
(12) Patent Application Publication
(10) Pub. No.: US 2008/0021026 A1
(43) Pub. Date: Jan. 24, 2008

(54) BENZOTHIOPHENE INHIBITORS OF RHO KINASE

(75) Inventors: Mehmet Kahraman, San Diego, CA (US); Allen J. Borchardt, San Diego, MO (US); Travis G. Cook, La Jolla, CA (US); Robert L. Davis, Carlsbad, CA (US); Elisabeth M.M. Gardiner, San Diego, CA (US); James W. Malecha, San Diego, CA (US); Stewart A. Noble, San Diego, CA (US); Thomas J. Prins, San Diego, CA (US)

Correspondence Address: GLOBAL PATENT GROUP - KAL;ATTN: MS LAVERN HALL, 10411 Clayton Road, Suite 304, St. Louis, MO, 63131, US

(73) Assignees: Mehmet Kahraman, San Diego, CA (US); Allen J. Borchardt, San Diego, CA (US); Travis G. Cook, La Jolla, CA (US); Robert L. Davis, Carlsbad, CA (US); Elizabeth M.M. Gardiner, San Diego, CA (US); James W. Malecha, San Diego, CA (US); Stewart A. Noble, San Diego, CA (US); Thomas J. Prins, San Diego, CA (US)

(21) Appl. No.: 11/780,834

(22) Filed: Jul. 20, 2007

Related U.S. Application Data
(60) Provisional application No. 60/832,634, filed on Jul. 20, 2006.
(60) Provisional application No. 60/915,772, filed on May 3, 2007.

(51) Int. Cl. A61K 31/44; A61K 31/415; A61K 31/497; A61K 31/535

(52) U.S. Cl. 514/233.5; 514/252.13; 514/275; 514/300; 514/337; 514/403

(57) ABSTRACT

The present invention relates to compounds and methods which may be useful as inhibitors of Rho kinase for the treatment or prevention of disease.

This application claims the benefit of priority of U.S. provisional application No. 60/832,634, filed Jul. 20, 2006 and U.S. provisional application No. 60/915,772, filed May 3, 2007, the disclosures of which is hereby incorporated by reference as if written herein in its entirety.

The present invention is directed to new benzothiophene compounds and compositions and their application as pharmaceuticals for the treatment of disease. Methods of inhibition of Rho kinase activity in a human or animal subject are also provided for the treatment of diseases such as opthalmologic diseases.

Many cell signaling events activate one or more members of the small monomeric GTPase superfamily. The Rho subfamily of GTPases (consisting of RhoA, RhoB, and RhoC) transmits signals, frequently from cell surface receptors, to effectors that play critical roles in control of cytoskeletal dynamics and gene regulation [Ridley, A. J., 2001, Trends Cell Biol. 11:471-477; Jaffe, A. B. and Hall, A., 2005, Annu Rev Cell Dev Biol. 21:247-269]. In particular, Rho-mediated effects on the cytoskeleton influence non-muscle cell shape, smooth muscle cell contraction, cell-cell and cell-matrix adhesion, intracellular vesicle transport, axonal and dendrite growth, vascular architecture, immune and inflammatory cell migration, and cleavage furrow formation and function during cell division [Bussey, H., 1996, Science. 272:224-225; Fukata, Y. et al., 2001, Trends Pharmacol Sci. 22:32-39; Luo, L., 2000, Nat Rev Neurosci. 1:173-180; Hu, E. and Lee, D., 2003, Curr Opin Investig Drugs. 4:1065-1075; Bokoch, G. M. 2005, Trends Cell Biol. 15:163-171; Wadsworth, P., 2005, Curr Biol. 15:R871-874].

Although the Rho GTPase cycle is complex, it can be briefly summarized as follows. Inactive, GDP-bound Rho, complexed with a GDP dissociation inhibitor protein (GDI), is recruited to the plasma membrane in response to signaling events, such as ligand binding to cell surface receptors. The GDI is displaced, whereby the inactive GDP-bound Rho is converted to active GTP-bound Rho by membrane-localized guanine-nucleotide exchange factors. GTP-bound Rho then binds and activates a number of effectors at the plasma membrane. Many proteins controlled by Rho activity have been identified, including a variety of protein and lipid kinases [Kaibuchi, K. et al., 1999, Annu Rev Biochem. 68:459-486; Bishop, A. L. and Hall, A., 2000, Biochem J. 348:241-255]. The intrinsic GTPase activity of Rho, stimulated by GTPase activating proteins, converts Rho back to the inactive, GDP-bound form, whereupon GDP-bound Rho can be extracted from the plasma membrane by the GDI (although in some instances, the GDI may extract GTP-bound Rho to extinguish a signal, or redirect GTP-bound Rho to a different compartment) [Sasaki T., and Takai Y., 1998, Biochem Biophys Res Commun. 245:641-645; Olofsson, B., 1999, Cell Signal. 11:545-554; Schmidt, A. and Hall, A., 2002, Genes Dev. 16:1587-1609; Moon, S. Y. and Zheng, Y., 2003, Trends Cell Biol. 13:13-22].

Of identified Rho effectors, the Rho-associated coiled-coil containing kinases, here referred to as Rho kinases, have been the subject of intense investigation in molecular and cell biological studies, and as pharmaceutical targets in multiple therapeutic areas. Rho kinases are serine-threonine protein kinases of approximately 160 kD molecular weight that contain an amino-terminal kinase catalytic domain, a long amphipathic alpha helical (coiled-coil) domain, an activated Rho binding domain, and a carboxy-terminal pleckstrin-homology domain (promoting binding to plasma membrane phosphoinositides) that is split by a cysteine rich zinc-finger like motif [Ishizaki, T., et al., 1996, EMBO J. 15, 1885-1893; Fujisawa, K. et al., 1996, J Biol Chem. 271:23022-23028; Matsui, T. et al., 1996, EMBO J. 15:2208-2216]. There are two known isoforms of Rho kinase, although splice variants may exist. These isoforms are referred to as Rho kinase (ROK) alpha (referred to here as ROCK2), and Rho kinase (ROK) beta, also known as p160 ROCK (referred to here as ROCK1) [Leung, T. et al., 1996, Mol Cell Biol. 16:5313-5327; Nakagawa, O. et al., 1996, FEBS Lett. 392:189-[93]. Many protein kinases are controlled by reversible phosphorylation events that switch them between active and inactive states. By contrast, Rho kinases switch from low, basal activity to high activity by reversible binding to GTP-bound Rho. Active Rho kinases then phosphorylate additional effectors of Rho signaling in the vicinity of the plasma membrane. Both Rho kinases are expressed in a mostly ubiquitous fashion in mammalian tissues at low to moderate levels, although expression is highly enriched in some cell types. Rho kinases share functional homology in their catalytic domains with the protein kinase A and C families, and a variety of small molecule inhibitors of Rho kinases also bind and inhibit protein kinase A in particular [Breitenlechner, C. et al., 2003, Structure. 11:1595-[607]. ROCK1 has 64% sequence identity to ROCK2 throughout the protein structure, and the kinase domains are highly conserved (90% identical).

As effectors of Rho signaling, Rho kinases are directly involved in controlling cytoskeleton dynamics, gene regulation, cell proliferation, cell division, and cell survival. Constitutively active mutants of Rho kinases can be generated by truncating carboxy-terminal regions, as far as the kinase domain, suggesting important negative regulation by the carboxy-terminal sequences. Expressed in cells, these mutants generate phenotypes consistent with hyperactive Rho kinase activity (e.g. increased stress fiber formation and cell-substrate focal adhesions). By contrast, deletion of the catalytic domain of Rho kinases results in a trans-dominant inhibitory effect in cells [Amano, M. et al., 1997, Science. 275:1308-1311; Leung, T. et al., 1996, Mol Cell Biol. 16:5313-5327; Amano, M. et al., 1999, J Biol Chem. 274:32418-32424]. There is data consistent with separable functions for ROCK1 and ROCK2 in cells, although these observations may be cell-type specific [Yoneda, A. et al., 2005, J Cell Biol. 170:443-453]. Although genetic knockout of ROCK1 leads to perinatal lethality due to omphaloceles in newborns, and genetic knockout of ROCK2 leads to a high incidence of embryonic lethality due to poor placental development, neither knockout alone is consistent with the necessity of ROCK1 or ROCK2 for most normal cell behaviors of the embryo during development [Shimizu, Y. et al., 2005, J Cell Biol. 168:941-953; Thumkeo, D. et al., 2003, Mol Cell Biol. 23:5043-5055].

Rho kinases can phosphorylate a variety of substrates to control various aspects of cytoskeletal behavior [Riento, K. and Ridley, A. J. 2003, Nat Rev Mol Cell Biol. 4:446-456]. Many studies have focused on control of the myosin light chain (MLC) regulatory subunit. Phosphorylation of the MLC regulatory subunit leads to increased actomyosin activity (e.g. smooth muscle cell contraction or increased non-muscle cell stress fibers). Rho kinases stimulate actomyosin activity by direct phosphorylation of the MLC regulatory subunit, and by inactivation of myosin light chain phosphatase through the phosphorylation of its myosin binding subunit [Amano, M. et al., 1996, J Biol Chem. 271:20246-20249; Kimura, K. et al., 1996, Science. 273:245-248; Kureishi, Y. et al., 1997, J Biol Chem. 272:12257-[2260]. LIM kinase, ezrin/radixin/moesin (ERM) family proteins, and adducin are some additional substrates of Rho kinases, and the phosphorylation of these and other proteins alters various aspects of cytoskeletal function [Oshiro, N., et al., 1998, J Biol Chem. 273:34663-34666; Kimura, K., et al., 1998, J Biol Chem. 273:5542-5548; Matsui, T., et al., 1998, J Cell Biol. 140:647-657; Fukata, Y., et al., 1999, J Cell Biol. 145:347-361; Kosako, H., et al., 1997, J Biol Chem. 272:10333-10336; Goto, H., et al., 1998, J Biol Chem. 273:11728-11736; Maekawa, M., et al., 1999, Science. 285:895-898; Ohashi, K., et al., 2000, J Biol Chem. 275:3577-3582].

Small molecule compounds such as Y-27632, Y-32885, Y-39983, HA-1077 (fasudil), hydroxy-fasudil, and a dimethylated analog of fasudil (H-1152P, or HMN-1152) have been demonstrated to directly inhibit Rho kinases. The Y compounds, which are more selective Rho kinase inhibitors, contain a common pyridine moiety, while fasudil and its analogs contain a common isoquinoline scaffold. Crystal structures for the kinase domain of ROCK1 complexed with Y-27632, fasudil, hydroxy-fasudil, and H-1152P have been reported (Jacobs, M. et al., 2006, J Biol Chem. 281:260-268]. All of these compounds occupy part of the ATP-binding pocket, consistent with the fact that they are reversible ATP competitive inhibitors.

These same Rho kinase inhibitors are cell permeable, and cause changes in cytoskeletal function and cell behavior consistent with loss of Rho kinase activity, similar to effects of the trans-dominant inhibitory mutants. Effects have been observed both in cultured cells in vitro and in physiologically responsive tissues in vivo [Nagumo, H. et al., 2000, Am J Physiol Cell Physiol. 278:C57-C65; Sinnett-Smith, J. et al., 2001, Exp Cell Res. 266:292-302; Chrissobolis, S. and Sobey, C. G., 2001, Circ Res. 88:774-779; Honjo, M. et al., 2001, Invest Opthalmol Vis Sci. 42:137-144; Takahara, A. et al., 2003, Eur J Pharmacol. 460:51-57; Fournier, A. E. et al., 2003, J Neurosci. 23:1416-1423; Rikitake, Y. et al., 2005, Stroke. 36:2251-2257; Slotta, J. E. et al. 2006, Inflamm Res. 55:364-367; Ying, H. et al., 2006, Mol Cancer Ther. 5:2158-2164]. The correlation between small molecule inhibition of Rho kinases and changes in cell behavior both in vitro and in vivo (e.g., vascular smooth muscle relaxation, bronchial smooth muscle relaxation, inhibition of immune and inflammatory cell migration, inhibition of tumor cell migration, inhibition of experimentally induced fibrosis, promotion of neural regenerative activity) supports the notion that Rho kinases are significant pharmaceutical targets for a wide range of therapeutic indications. In addition, it is now more appreciated that some of the “pleiotropic” and beneficial cardiovascular effects of clinically useful HMG Coenzyme A reductase inhibitors (i.e., the “statin” drug class) are a consequence of decreased Rho, and therefore decreased Rho kinase, activity, especially in endothelial cells [Eto, M. et al., 2002, Circulation. 105:1756-1759; Rikitake, Y. and Liao, J. K., 2005, Circ Res. 97:1232-1235; Kozai, T. et al., 2005, Cardiovasc Res. 68:475-482; Girgis, R. E. et al., 2007, Am J Physiol Lung Cell Mol Physiol. 292:L1105-L1110]. Interestingly, Rho kinase inhibition has been recently implicated in the enhanced survival and cloning efficiency of dissociated human embryonic stem cells, which suggests the utility of Rho kinase inhibitors for stem cell therapies [Watanabe, K. et al., 2007, Nat Biotechnol. 25:681-686].

Novel compounds and pharmaceutical compositions, certain of which have been found to inhibit Rho kinase have been discovered, together with methods of synthesizing and using the compounds including methods for the treatment of Rho kinase-mediated diseases in a patient by administering the compounds.

The present invention discloses a class of compounds, certain of which may be useful in treating Rho kinase-mediated disorders and conditions, defined by structural Formula I:

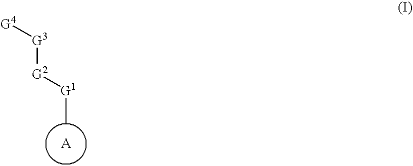

A is optionally substituted heteroaryl;

G¹ is optionally substituted fused bicyclic heteroaryl;

G² is selected from the group consisting of (CR^{a}R^{b})ₘZ(CR^{c}R^{d})ₚ and null;

m and p are independently 0, 1, 2, 3, or 4;

Z is selected from the group consisting of O, N(R¹), S(O)ₙ, N(R^{e})CO, CON(R^{e}), N(R^{e})SO₂, SO₂N(R^{e}), C(O), optionally substituted cycloalkyl, and null;

R^{e} is selected from the group consisting of hydrogen and optionally substituted C₁-C₄ alkyl;

n is 0, 1 or 2;

R^{a}, R^{b}, R^{c}, and R^{d} are independently selected from the group consisting of hydrogen, alkyl, amino, aminoalkyl, amidoalkyl, aminoalkylcarboxyl, carboxylalkyl, halo, heterocycloalkyl, heterocycloalkylalkyl, hydroxyalkyl, heteroarylalkyl and heterocycloalkylalkylcarboxyl;

G³ is selected from the group consisting of lower alkyl, cycloalkyl, aryl, arylalkyl, heterocycloalkyl, heteroaryl, lower alkoxy, lower alkylthio, acyl, carboxyl, sulfonamide, hydroxy, and null, any of which may be optionally substituted;

G⁴ is selected from the group consisting of hydrogen, halogen, alkyl, alkoxy, amino, aminoalkyl, amido, amidoalkyl, alkylamido, aminoalkylcarboxyl, carboxyl, alkylcarboxyl, cycloalkyl, heterocycloalkyl, heterocycloalkylcarbonyl, heterocycloalkylalkyl, heterocycloalkylalkoxy, heterocycloalkylalkylcarboxy, heterocycloalkylalkylamido, aryl, arylalkoxy, arylamido, arylalkyl, arylacyl, arylcarboxy, heteroarylalkyl, and urea, any of which may be optionally substituted; and

R¹ is selected from the group consisting of alkyl, alkylcarbonyl, alkylene, alkynyl, amino, alkylamino, carbonyl, cycloalkyl, ester, heterocycloalkyl, heterocycloalkylalkyl, heteroalkyl, and hydrogen, any of which may be optionally substituted.

Certain compounds according to the present invention possess useful Rho kinase inhibiting activity, and may be used in the treatment or prophylaxis of a disease or condition in which Rho kinase plays an active role. Thus, in broad aspect, the certain embodiments of the present invention also provide pharmaceutical compositions comprising one or more compounds disclosed herein together with a pharmaceutically acceptable carrier, as well as methods of making and using the compounds and compositions. Certain embodiments of the present invention provide methods for inhibiting Rho kinase. Other embodiments of the present invention provide methods for treating a Rho kinase-mediated disorder in a patient in need of such treatment, comprising administering to said patient a therapeutically effective amount of a compound or composition according to the present invention. The present invention also contemplates the use of certain compounds disclosed herein for use in the manufacture of a medicament for the treatment of a disease or condition ameliorated by the inhibition Rho kinase.

In further embodiments, A is selected from the group consisting of optionally substituted monocyclic 5 to 6 membered heteroaryl containing at least one ring nitrogen, or an optionally substituted bicyclic heteroaryl which comprises a five-membered ring fused to a six-membered ring and which contains at least one ring nitrogen.

In yet further embodiments, G¹ is selected from the group consisting of:

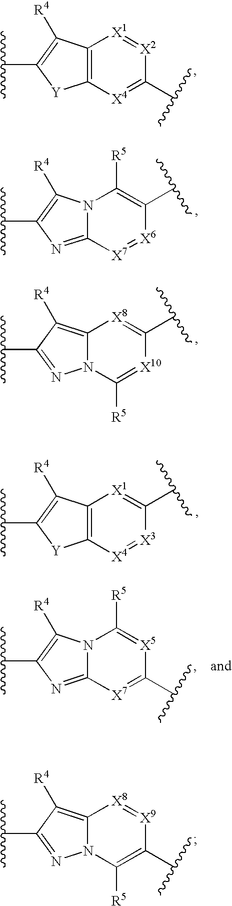

X¹ is N or C(R⁶);

X² is N or C(R⁷);

X³ is N or C(R⁸);

X⁴ is N or C(R⁹);

X⁵ is N or C(R¹⁰);

X⁶ is N or C(R¹¹);

X⁷ is N or C(R¹²);

X⁸ is N or C(R¹³);

X⁹ is N or C(R¹⁴);

X¹⁰ is N or C(R¹⁵);

Y is O or S; and

R⁴-R¹⁵ are independently selected from the group consisting of hydrogen, halogen, lower alkyl, cycloalkyl, aryl, heterocycloalkyl, heteroaryl, lower alkoxy, lower alkylthio, lower haloalkyl, acyl, amino, carboxyl, cyano, and nitro, any of which may be optionally substituted.

In yet further embodiments, A is selected from the group consisting of

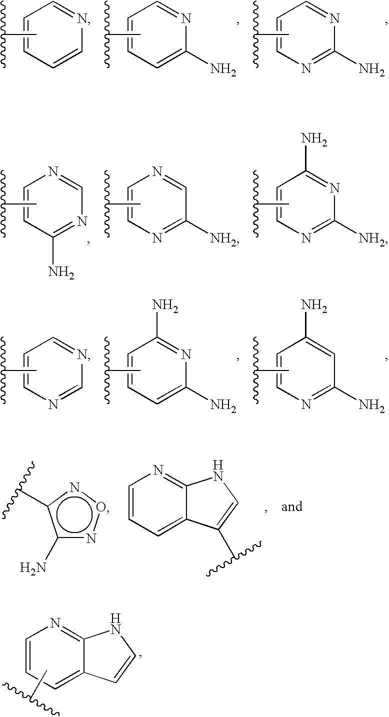

any of which may be optionally substituted.

In yet further embodiments,

G² is (CR^{a}R^{b})ₘZ(CR^{c}R^{d})ₚ;

m and p are independently 0, 1, or 2;

Z is selected from the group consisting of O, N(R¹), S(O)ₙ, N(R^{e})CO, CON(R^{e}), C(O), and null;

R^{e} is selected from the group consisting of hydrogen and optionally substituted C₁-C₄ alkyl; and

n is 0 or 2.

In yet further embodiments, wherein G¹ is:

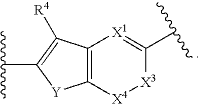

In yet further embodiments, A is selected from the group consisting of

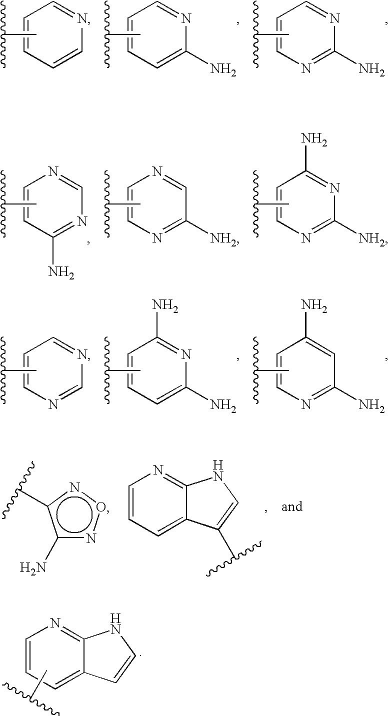

In yet further embodiments, the compounds of the present invention have structural Formula II

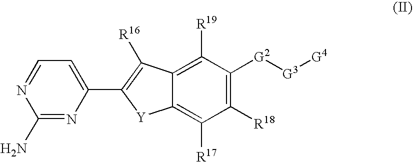

wherein:

Y is O or S;

G² is (CR^{a}R^{b})ₘZ(CR^{c}R^{d})ₚ;

m and p are independently 0, 1, or 2;

Z is selected from the group consisting of O, N(R¹), S(O)ₙ, N(R^{e})CO, CON(R^{e}), C(O), and null;

R^{e} is selected from the group consisting of hydrogen and optionally substituted C₁-C₄ alkyl; and

n is 0 or 2;

G³ is selected from the group consisting of lower alkyl, cycloalkyl, aryl, arylalkyl, heterocycloalkyl, heteroaryl, lower alkoxy, lower alkylthio, acyl, carboxyl, sulfonamide, hydroxy, and null, any of which may be optionally substituted;

G⁴ is selected from the group consisting of hydrogen, halogen, alkyl, alkoxy, amino, aminoalkyl, amido, amidoalkyl, alkylamido, aminoalkylcarboxyl, carboxyl, alkylcarboxyl, cycloalkyl, heterocycloalkyl, heterocycloalkylcarbonyl, heterocycloalkylalkyl, heterocycloalkylalkoxy, heterocycloalkylalkylcarboxy, heterocycloalkylalkylamido, aryl, arylalkoxy, arylamido, arylalkyl, arylacyl, arylcarboxy, heteroarylalkyl, and urea, any of which may be optionally substituted;

R¹⁶ is selected from the group consisting of lower alkenyl, alkynyl, lower alkyl, alkylthio, haloalkyl, heteroalkyl, hydroxyalkyl, halogen, and hydrogen; and

R¹⁷-R¹⁹ are independently selected from the group consisting of acyl, lower alkenyl, alkynyl, lower alkoxy, lower alkoxyalkyl, lower alkyl, alkylthio, amido, amino, aminoalkyl, aminocarbonyl, carboxyl, haloalkyl, hydroxyalkyl and hydrogen, any of which may be optionally substituted.

In yet further embodiments,

Y is S;

R¹⁶ is selected from the group consisting of lower alkyl and hydrogen; and

R¹⁷-R¹⁹ are all hydrogen.

In yet further embodiments, G³ is selected from the group consisting of aryl, heterocycloalkyl, heteroaryl, any of which may be optionally substituted.

In yet further embodiments, either

m and p are both 0; and

Z is selected from the group consisting of O, NH, S, and C(O); or

m is 1;

Z is null; and

p is 0.

In yet further embodiments, R¹⁶ is selected from the group consisting of methyl, ethyl, heteroalkyl, and halogen.

In yet further embodiments, G⁴ is selected from the group consisting of hydrogen, halogen, alkoxy, amino, alkylamido, carboxyl, alkylcarboxyl, heterocycloalkylalkyl, heterocycloalkylalkoxy, heterocycloalkylalkylcarboxy, and heterocycloalkylalkylamido, any of which may be optionally substituted.

In certain further embodiments, compounds of structural Formulas I-IV may find use in the inhibition of Rho kinase for the treatment of disease.

In certain further embodiments, compounds of structural Formulas I-IV may be administered in combination with at least one other therapeutic agent.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycloalkyl, or any other moiety were the atom attached to the carbonyl is carbon. An “acetyl” group, which is a type of acyl, refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether

(R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—N(R)₂ group with R as defined herein. The term “N-amido” as used herein, alone or in combination, refers to a RC(═O)N(R′)— group, with R and R′ as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —N(R)(R′) or —N⁺(R)(R′)(R″), wherein R, R′ and R″ are independently selected from the group consisting of hydrogen, alkyl, acyl, heteroalkyl, aryl, cycloalkyl, heteroaryl, and heterocycloalkyl, any of which may themselves be optionally substituted.

The term “amino acid,” as used herein, alone or in combination, means a substituent of the form —NRCH(R′)C(O)OH, wherein R is typically hydrogen, but may be cyclized with N (for example, as in the case of the amino acid proline), and R′ is selected from the group consisting of hydrogen, alkyl, heteroalkyl, cycloalkyl, heterocycloalkyl, aryl, heteroaryl, amino, amido, cycloalkylalkyl, heterocycloalkylalkyl, arylalkyl, heteroarylalkyl, aminoalkyl, amidoalkyl, hydroxyalkyl, thiol, thioalkyl, alkylthioalkyl, and alkylthio, any of which may be optionally substituted. The term “amino acid” includes all naturally occurring amino acids as well as synthetic analogues.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxyl,” as used herein, refers to —C(O)OH, O-carboxy, C-carboxy, or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxyl group bridging two moieties linked at carbon atoms.

The term “ether,” as used herein, alone or in combination, typically refers to an oxy group bridging two moieties linked at carbon atoms. “Ether” may also include polyethers, such as, for example, —RO(CH₂)₂O(CH₂)₂O(CH₂)₂OR′, —RO(CH₂)₂O(CH₂)₂OR′, —RO(CH₂)₂OR′, and —RO(CH₂)₂OH.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCI—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃. The term heteroalkyl may include ethers.

The term “heteroaryl,” as used herein, alone or in combination, refers to 3 to 7 membered unsaturated heteromonocyclic rings, or fused polycyclic rings in which at least one of the fused rings is unsaturated, wherein at least one atom is selected from the group consisting of O, S, and N. In certain embodiments, said heteroaryl will comprise from 5 to 7 carbon atoms. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one heteroatom as ring members, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sugars, sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycloalkyl groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycloalkyl groups may be optionally substituted unless specifically prohibited.

The term “hydrazinyl” as used herein, alone or in combination, refers to two amino groups joined by a single bond, i.e., —N—N—.

The term “hydroxamic acid” as used herein, refers to —C(O)ON(R)O(R′), wherein R and R′ are as defined herein, or the corresponding “hydroxamate” anion, including any corresponding hydroxamic acid salt. Hydroxamate also includes reverse hydroxamates of the form —ON(R)O(O)CR′.

The term “hydroxy,” or, equivalently, “hydroxyl,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “phosphoamide” as used herein, alone or in combination, refers to a phosphate group [(OH)₂P(O)O—] in which one or more of the hydroxyl groups has been replaced by nitrogen, amino, or amido.

The term “phosphonate” as used herein, alone or in combination, refers to a group of the form ROP(OR′)(OR)O— wherein R and R′ are selected from the group consisting of hydrogen, alkyl, acyl, heteroalkyl, aryl, cycloalkyl, heteroaryl, and heterocycloalkyl, any of which may themselves be optionally substituted. “Phosphonate” includes “phosphate [(OH)₂P(O)O—] and related phosphoric acid anions which may form salts.

The terms “sulfonate,” “sulfonic acid,” and “sulfonic,” as used herein, alone or in combination, refers to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′-group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent. A “null” group occurring between two other groups may also be understood to be a collapsing of flanking groups. For example, if in —(CH₂)ₛG¹G²G³, the element G² were null, said group would become —(CH₂)ₛG¹G³.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, hydroxyl, halogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers, including keto-enol tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the body or of one of its parts that impairs normal functioning and is typically manifested by distinguishing signs and symptoms.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“Rho kinase inhibitor” is used herein to refer to a compound that exhibits an IC₅₀ with respect to Rho kinase activity of no more than about 100 μM and more typically not more than about 50 μM, as measured in the Rho kinase assay described generally hereinbelow. “IC₅₀” is that concentration of inhibitor which reduces the activity of an enzyme (e.g., Rho kinase) to half-maximal level. Certain representative compounds of the present invention have been discovered to exhibit inhibition against Rho kinase. In certain embodiments, compounds will exhibit an IC₅₀ with respect to Rho kinase of no more than about 10 μM; in further embodiments, compounds will exhibit an IC₅₀ with respect to Rho kinase of no more than about 5 μM; in yet further embodiments, compounds will exhibit an IC₅₀ with respect to Rho kinase of not more than about 1 μM, as measured in the Rho kinase assay described herein. In yet further embodiments, compounds will exhibit an IC₅₀ with respect to Rho kinase of not more than about 200 nM.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain of the present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The compounds of the present invention can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds of the present invention which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxyl group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds of the present invention, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating Rho kinase-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of Rho kinase-mediated disorders.

Compounds of the subject invention may be useful in treating Rho kinase-mediated disease, disorders and conditions. In certain embodiments, said compounds may find use in treating acute and chronic pain and inflammation. The compounds of the present invention may be useful to treat patients with neuropathy, neuropathic pain, or inflammatory pain such as reflex sympathetic dystrophy/causalgia (nerve injury), peripheral neuropathy (including diabetic neuropathy), intractable cancer pain, complex regional pain syndrome, and entrapment neuropathy (carpel tunnel syndrome). The compounds may also be useful in the treatment of pain associated with acute herpes zoster (shingles), postherpetic neuralgia (PHN), and associated pain syndromes such as ocular pain. The compounds may further be useful as analgesics in the treatment of pain such as surgical analgesia, or as an antipyretic for the treatment of fever. Pain indications include, but are not limited to, post-surgical pain for various surgical procedures including post-cardiac surgery, dental pain/dental extraction, pain resulting from cancer, muscular pain, mastalgia, pain resulting from dermal injuries, lower back pain, headaches of various etiologies, including migraine, and the like. The compounds may also be useful for the treatment of pain-related disorders such as tactile allodynia and hyperalgesia. The pain may be somatogenic (either nociceptive or neuropathic), acute and/or chronic. The Rho kinase inhibitors of the subject invention may also be useful in conditions where NSAIDs, morphine or fentanyl opiates and/or other opioid analgesics would traditionally be administered.

Furthermore, compounds of the subject invention may be used in the treatment or prevention of opiate tolerance in patients needing protracted opiate analgesics, and benzodiazepine tolerance in patients taking benzodiazepines, and other addictive behavior, for example, nicotine addiction, alcoholism, and eating disorders. Moreover, the compounds and methods of the present invention may be useful in the treatment or prevention of drug withdrawal symptoms, for example treatment or prevention of symptoms of withdrawal from opiate, alcohol, or tobacco addiction.

In addition, compounds of the subject invention may be used to treat insulin resistance and other metabolic disorders such as atherosclerosis that are typically associated with an exaggerated inflammatory signaling.

The present invention encompasses therapeutic methods using novel selective Rho kinase inhibitors to treat or prevent respiratory disease or conditions, including therapeutic methods of use in medicine for preventing and treating a respiratory disease or condition including: asthmatic conditions including allergen-induced asthma, exercise-induced asthma, pollution-induced asthma, cold-induced asthma, and viral-induced-asthma; asthma-related diseases such as airway hyperreactivity and small airway disease; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchiolitis, bronchioectasis, cystic fibrosis, pigeon fancier's disease, farmer's lung, acute respiratory distress syndrome, pneumonia, pneumonitis, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus, hypoxia, dyspnea, hypercapnea, hyperinflation, hypoxemia, and cough. Further, compounds disclosed herein would find use in the treatment of allergic disorders such as delayed type hypersensitivity reaction, allergic contact dermatitis, allergic rhinitis, and chronic sinusitis.

Other disorders or conditions which may be treated by the compounds of the present invention include inflammation and related disorders. The compounds of the present invention may be useful as anti-inflammatory agents with the additional benefit of having significantly less harmful side effects. The compounds may be useful to treat arthritis, including but not limited to rheumatoid arthritis, spondyloarthropathies, gouty arthritis, osteoarthritis, juvenile arthritis, acute rheumatic arthritis, enteropathic arthritis, neuropathic arthritis, psoriatic arthritis, reactive arthritis (Reiter's syndrome), and pyogenic arthritis, and autoimmune diseases, including systemic lupus erythematosus, hemolytic syndromes, autoimmune hepatitis, autoimmune neuropathy, vitiglio (autoimmune thyroiditis), Hashimoto's thyroiditis, anemias, myositis including polymyositis, alopecia greata, Goodpasture's syndrome, hypophytis, and pulmonary fibrosis.

The compounds may also be useful in treating osteoporosis and other related bone disorders.

These compounds may also be used to treat gastrointestinal conditions such as reflux esophagitis, diarrhea, inflammatory bowel disease, Crohn's disease, gastritis, irritable bowel syndrome, Graves' disease (hyperthyroidism), necrotizing enterocolitis, and ulcerative colitis. The compounds may also be used in the treatment of pulmonary inflammation, such as that associated with viral infections and cystic fibrosis.

In addition, compounds of invention may also be useful in organ transplant patients either alone or in combination with conventional immunomodulators. Examples of conditions to be treated in said patients include graft vs. host reaction (i.e., graft vs. host disease), allograft rejections (e.g., acute allograft rejection, and chronic allograft rejection), transplant reperfusion injury, and early transplantation rejection (e.g., acute allograft rejection).

Yet further, the compounds of the invention may be useful in the treatment of pruritis and vitaligo.

The compounds of the present invention may also be useful in treating tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephritis, nephrotic syndrome, Langerhans' cell histiocytosis, glomerulonephritis, reperfusion injury, pancreatitis, interstitial cystitis, Behcet's syndrome, polymyositis, gingivitis, periodontis, hypersensitivity, swelling occurring after injury, ischemias including myocardial ischemia, cardiovascular ischemia, and ischemia secondary to cardiac arrest, cirrhosis, septic shock, endotoxic shock, gram negative sepsis, toxic shock syndrome, stroke, ischemia reperfusion injury, multi-organ dysfunction, restenosis including restenosis following coronary bypass surgery, and the like.

The compounds of the subject invention may also be useful for the treatment of certain diseases and disorders of the nervous system. Central nervous system disorders in which Rho kinase inhibition may be useful include cortical dementias including Alzheimer's disease and mild cognitive impairment (MCI), central nervous system damage resulting from stroke, ischemias including cerebral ischemia (both focal ischemia, thrombotic stroke and global ischemia (for example, secondary to cardiac arrest), and trauma. Neurodegenerative disorders in which Rho kinase inhibition may be useful include nerve degeneration or nerve necrosis in disorders such as hypoxia, hypoglycemia, epilepsy, and in cases of central nervous system (CNS) trauma (such as spinal cord and head injury), hyperbaric oxygen convulsions and toxicity, dementia (e.g. pre-senile dementia), and AIDS-related dementia, cachexia, Sydenham's chorea, Huntington's disease, Parkinson's Disease, amyotrophic lateral sclerosis (ALS), multiple sclerosis, Korsakoff's syndrome, and imbecility relating to a cerebral vessel disorder. Further disorders in which Rho kinase inhibition might prove useful include neuropathies of the central and peripheral nervous system (including, for example, IgA neuropathy, membranous neuropathy and idiopathic neuropathy), chronic inflammatory demyelinating polyneuropathy, transverse myelitis, Gullain-Barre disease, encephalitis, and cancers of the nervous system. Disorders of CNS function in which Rho kinase inhibitors may find use include sleeping disorders, schizophrenia, depression, depression or other symptoms associated with Premenstrual Syndrome (PMS), and anxiety.

Furthermore, the compounds of the present invention may also be useful in inhibiting Rho kinase activity for the amelioration of systemic disorders including septic and/or toxic hemorrhagic shock induced by a wide variety of agents; as a therapy with cytokines such as TNF, IL-1 and IL-2; and as an adjuvant to short term immunosuppression in transplant therapy.

Still other disorders or conditions which may be treated by the compounds of the subject invention include the prevention or treatment of cancer, such as colorectal cancer, and cancer of the breast, lung, prostate, bladder, cervix and skin. Compounds of the invention may be used in the treatment and prevention of neoplasias including but not limited to brain cancer, bone cancer, leukemia, lymphoma, epithelial cell-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. The neoplasia can be selected from gastrointestinal cancer, liver cancer, bladder cancer, pancreas cancer, ovary cancer, prostate cancer, cervical cancer, lung cancer, breast cancer and skin cancer, such as squamous cell and basal cell cancers. The present compounds and methods may also be used to treat the fibrosis which occurs with radiation therapy. The present compounds and methods may be used to treat subjects having adenomatous polyps, including those with familial adenomatous polyposis (FAP). Additionally, the present compounds and methods may be used to prevent polyps from forming in patients at risk of FAP.

The compounds of the subject invention may be used in the treatment of ophthalmic diseases, such as dry eye, glaucoma, corneal neovascularization, optic neuritis, Sjogren's syndrome, retinal ganglion degeneration, ocular ischemia, retinitis, retinopathies, uveitis, ocular photophobia, and of inflammation and pain associated with acute injury to the eye tissue. Specifically, the compounds may be used to treat glaucomatous retinopathy and/or diabetic retinopathy. The compounds may also be used to treat post-operative inflammation or pain as from ophthalmic surgery such as cataract surgery and refractive surgery.

The compounds of the subject invention may be used in the treatment of menstrual cramps, dysmenorrhea, premature labor, endometriosis, tendonitis, bursitis, skin-related conditions such as psoriasis, eczema, burns, sunburn, dermatitis, pancreatitis, hepatitis, lichen planus, scleritis, scleroderma, dermatomyositis, and the like. Other conditions in which the compounds of the subject invention may be used include diabetes (type I or type II), myocarditis, pathological angiogenesis, and aortic aneurysm.

Moreover, compounds of the subject invention may be used in the treatment of cardiovascular disease, such as angina, coronary artery vasospasm, myocardial infarction, coronary ischemia, congestive heart failure, cardiac allograft vasculopathy, vein graft disease and vascular restenosis, ischemic reperfusion injury, cerebral artery vasospasm, stroke, cerebral ischemia, essential hypertension, pulmonary hypertension, renal hypertension and other secondary hypertensive disorders, atherosclerosis and erectile dysfunction.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds of the subject invention may also be used to prevent tissue damage when therapeutically combined with antibacterial or antiviral agents.

Differentiated cells produced from hES cells may be useful for treating degenerative diseases whose symptoms are caused by loss of a few particular cell types. Specific types of neurons have been generated from mouse ES (mES) cells, and similar selective differentiation methods have been applied to hES cells. However, hES cells have been technically much harder to culture than mES cells, showing problematic properties such as slow growth and insensitivity to the trophic substance leukemia inhibitory factor (LIF). In addition, hES cells are vulnerable to apoptosis upon cellular detachment and dissociation. They undergo massive cell death particularly after complete dissociation, and the cloning efficiency of dissociated hES cells is generally ≦1%. Thus, hES cells are difficult, if not impossible, to use in dissociation culture, which is important for such procedures as clonal isolation following gene transfer and differentiation induction. Poor survival of human embryonic stem (hES) cells after cell dissociation is an obstacle to research, hindering manipulations such as subcloning.

Recent evidence suggests that addition of selective inhibitors of Rho kinase may enable hES cells to grow and differentiate as mES cells do under unfavorable culture conditions such as dissociation and suspension. Rho kinase inhibition has been shown to markedly diminish dissociation-induced apoptosis, increase cloning efficiency (from about 1% to about 27%) and facilitate subcloning after gene transfer in hES cells. The improvement in cloning efficiency conferred Rho kinase inhibition may be particularly advantageous for isolating relatively rare clones (e.g., those for homologous recombination) and also for recloning hES cells to obtain a uniform cell quality. Furthermore, dissociated hES cells treated with selective inhibitors of Rho kinase are protected from apoptosis even in serum-free suspension (SFEB) culture, form floating aggregates, and survive and differentiate, as do SFEB-cultured mouse ES cells.

Many methods exist for the production or derivation of hES cells. For example, histocompatible parthenogenetic human embryonic stem cells (phESC) may be derived from human parthenogenetic blastocysts. The utility of Rho kinase inhibitors disclosed above, and the methods below, would be expected to be applicable to any hES cells demonstrating typical hES cell morphology and/or properties, regardless of origin.

Accordingly, the invention contemplates the use of certain compounds and compositions disclosed herein: for reduction of apoptosis of human embryonic stem cells; for increasing survival of human embryonic stem cells; for increasing cloning efficiency of human embryonic stem cells after gene transfer; and for enhancing differentiation of cultured human embryonic stem cells. In further embodiments, said prevention of apoptosis of human embryonic stem cells and/or said increasing of survival of human embryonic stem cells occurs in dissociated culture, such as, for example, serum-free suspension (SFEB) culture.

Besides being useful for human treatment, the compounds and formulations of the present invention are also useful for veterinary treatment of companion animals, exotic animals and farm animals, including mammals, rodents, and the like. More preferred animals include horses, dogs, and cats.

### General Synthetic Methods for Preparing Compounds

The following schemes can be used to practice the present invention.

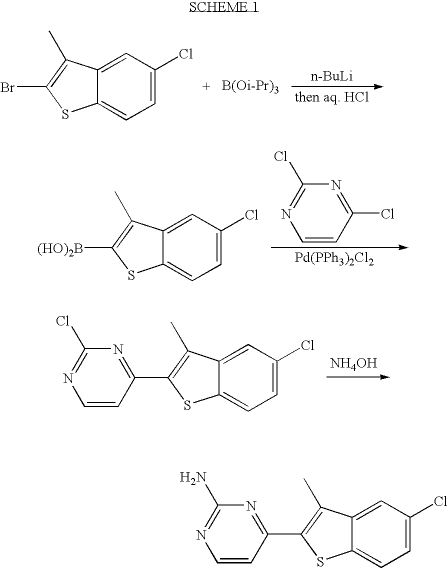

Examples 1-2 can be synthesized using the following general synthetic procedure set forth in Scheme 1.

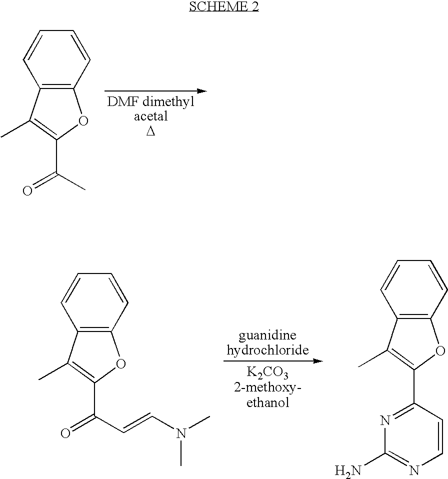

Examples 3-12 can be synthesized using the following general synthetic procedure set forth in Scheme 2.

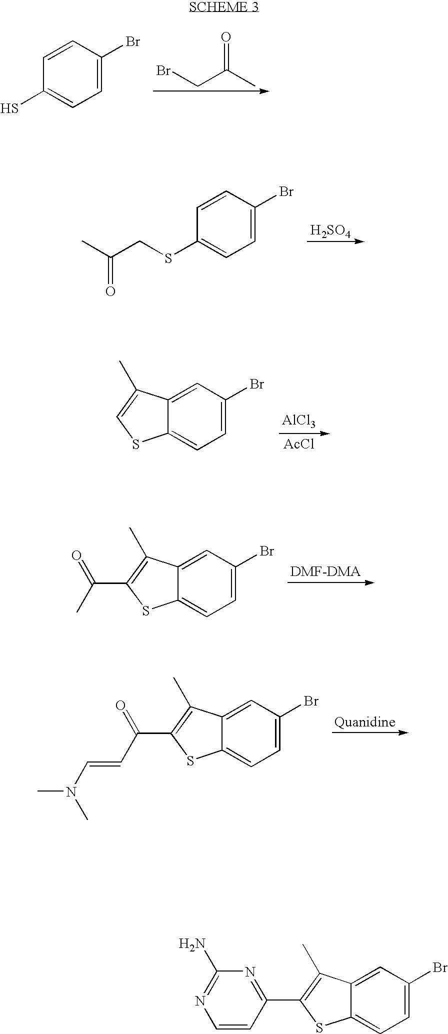

Examples 13-14 can be synthesized using the following general synthetic procedure set forth in Scheme 3.

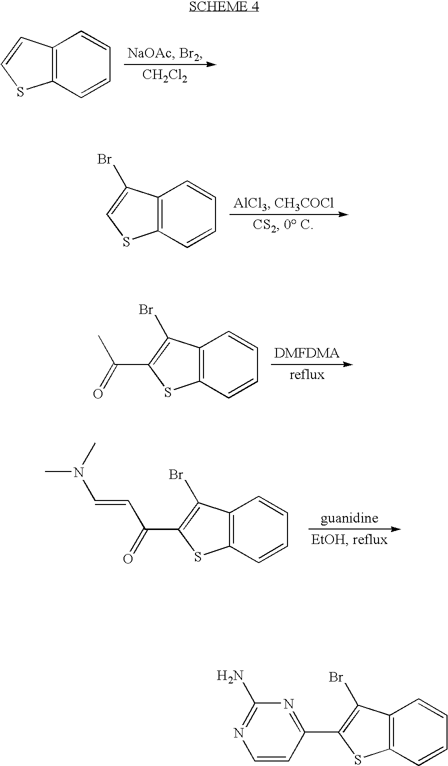

Example 15 can be synthesized using the following general synthetic procedure set forth in Scheme 4.

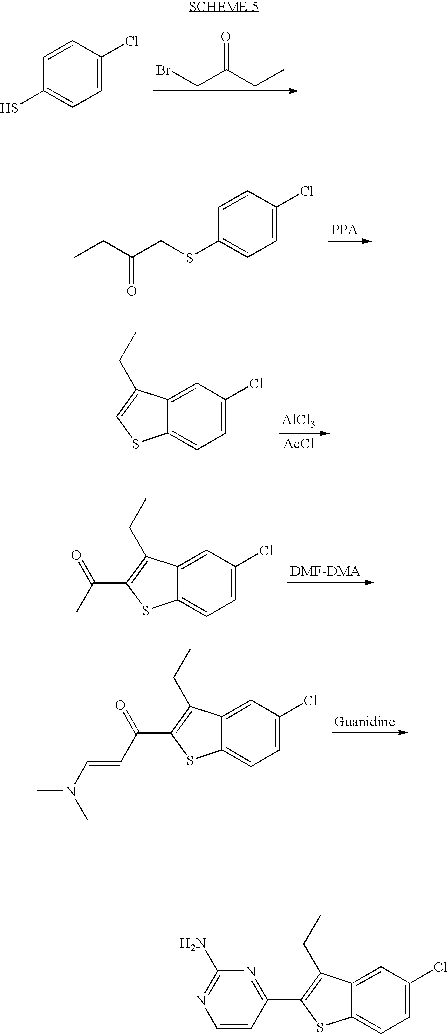

Example 16 can be synthesized using the following general synthetic procedure set forth in Scheme 5.

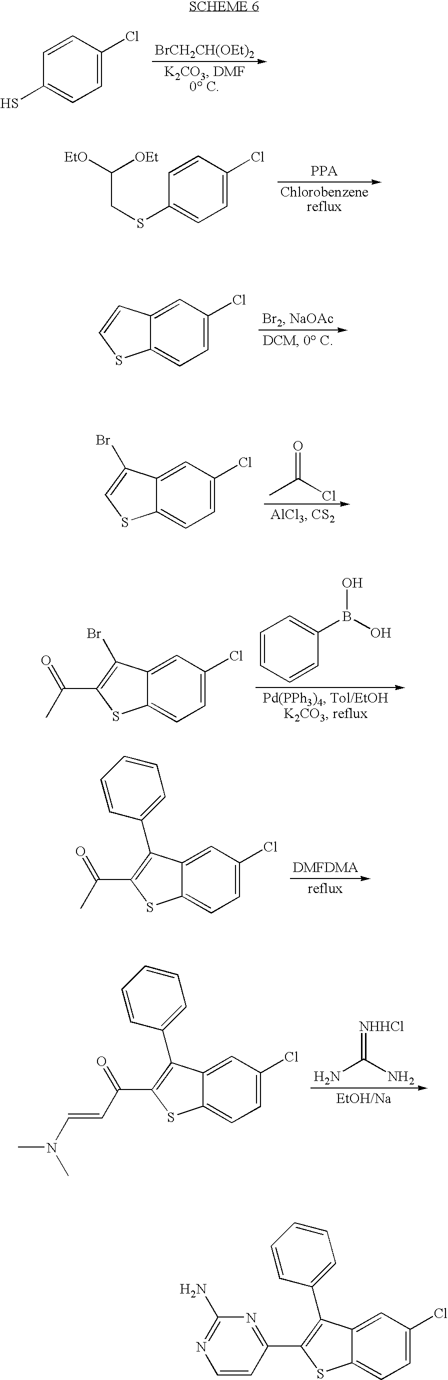

Example 17 can be synthesized using the following general synthetic procedure set forth in Scheme 6.

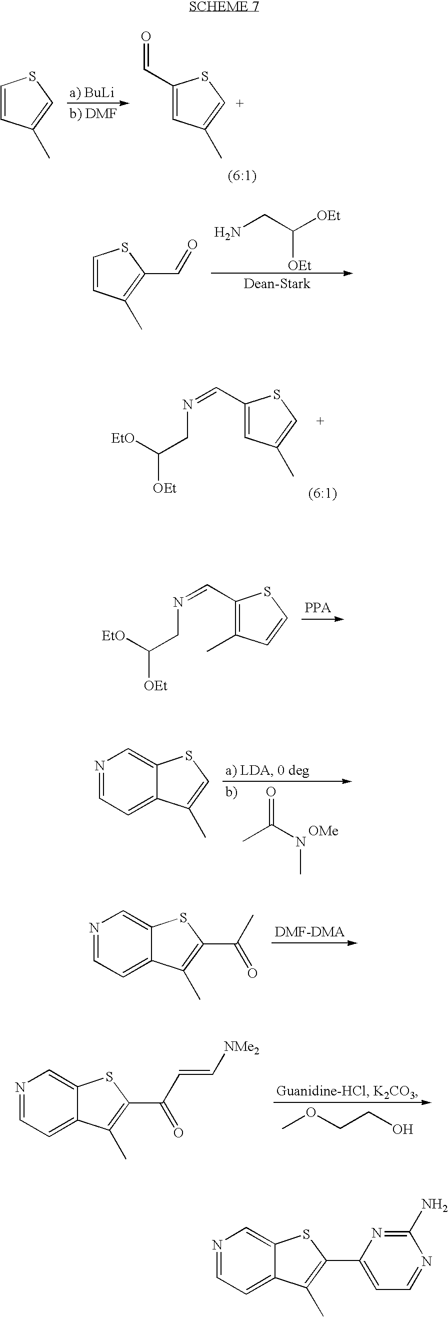

Examples 18-28 can be synthesized using the following general synthetic procedure set forth in Scheme 7.

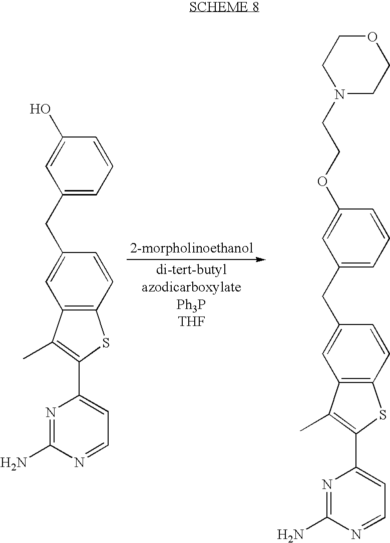

Examples 29-31 can be synthesized using the following general synthetic procedure set forth in Scheme 8.

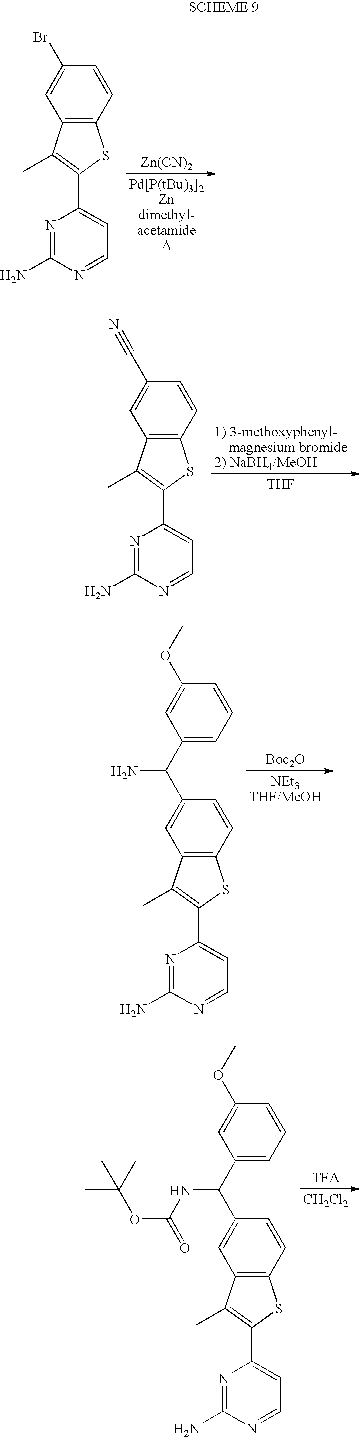

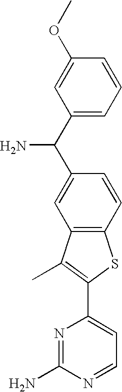

Examples 32-77 can be synthesized using the following general synthetic procedure set forth in Scheme 9.

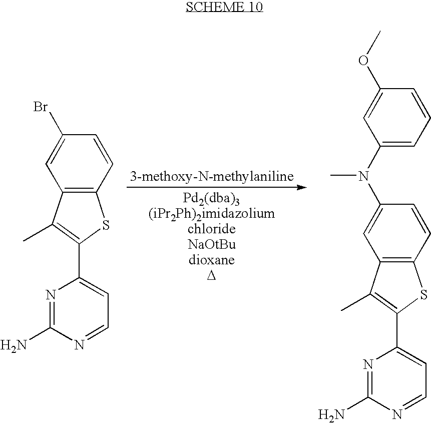

Example 78 can be synthesized using the following general synthetic procedure set forth in Scheme 10.

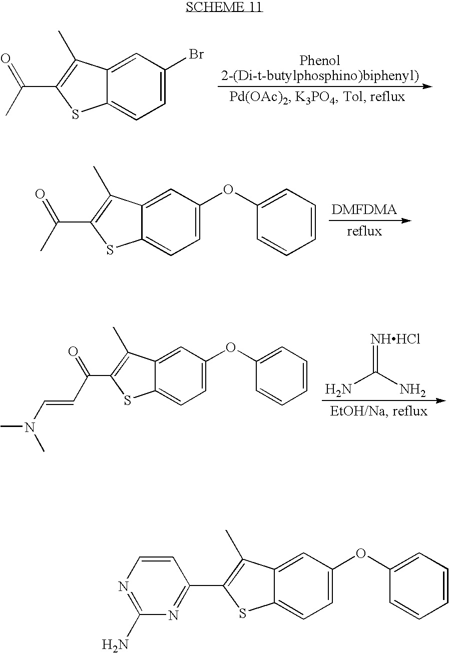

Examples 79-90 can be synthesized using the following general synthetic procedure set forth in Scheme 11.

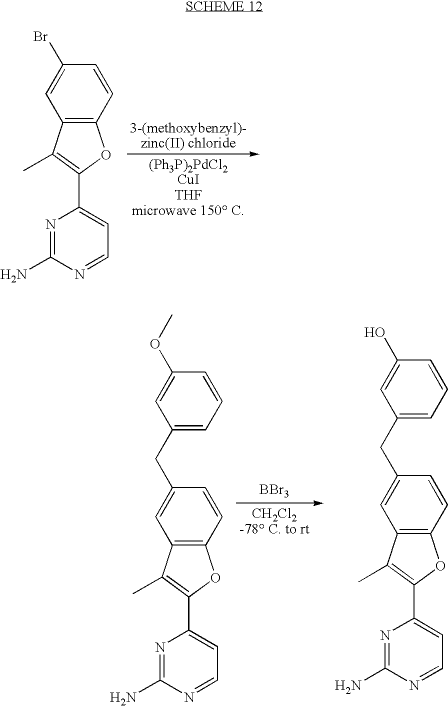

Example 92 can be synthesized using the following general synthetic procedure set forth in Scheme 12.

The invention is further illustrated by the following examples.

### EXAMPLE 1

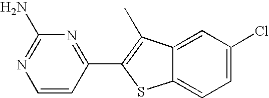

### 4-(5-Chloro-3-methylbenzo[b]thiophen-2-yl)pyrimidin-2-amine

Step 1

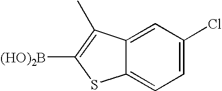

### 5-Chloro-3-methylbenzo[b]thiophen-2-ylboronic acid

To a solution of 2-bromo-5-chloro-3-methylbenzo[b]thiophene (1 g, 3.8 mmol), and triisopropyl borate (0.85 g, 4.56 mmol) in 4:1 THF/toluene, was added n-butyllithium (4.56 mol, 2.8 mL of 1.6M solution in hexanes) at −78° C. over 15 minutes. The mixture was gradually warmed to room temperature, and stirred for 30 min. The reaction was quenched by addition of an aqueous solution of hydrochloric acid (2M) while stirring vigorously for 10 minutes. The reaction mixture was diluted with THF followed by addition of solid NaCl (10 g). The mixture was extracted with EtOAc, washed with water, brine, dried over Na₂SO₄, and filtered. The filtrate was concentrated, and the crude product was purified by silica gel column chromatography eluted with 10% methanol in methylene chloride to afford 0.73 g (85% yield) as a off-white solid. ¹H NMR (400 MHz, DMSO-d₆) δ: 7.99 (d, 1H), 7.87 (d, 1H), 7.39 (dd, 1H), 2.73 (s, 3H).
Step 2

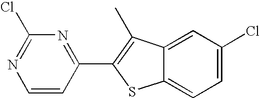

### 2-Chloro-4-(5-chloro-3-methylbenzo[b]thiophen-2-yl)pyrimidine

To a solution of 5-chloro-3-methylbenzo[b]thiophen-2-ylboronic acid (0.3 g, 1.3 mmol), and 2,4-dichloropyrimidine (0.2 g, 1.3 mmol) in 3:1 THF/water, was added an aqueous solution of Na₂CO₃ (1.6 mL, 2M). The mixture was degassed three times and back filled with nitrogen, followed by the addition of Pd(Ph₃P)₂Cl₂ (0.091 g 0.13 mmol) in one portion. The reaction mixture was then heated to 70° C. for 2 hours. LCMS confirmed the completion of the reaction. The vessel was cooled down to room temperature, and diluted with ethyl acetate (100 mL). The organic layer was washed with water, brine, dried over Na₂SO₄, and filtered. The filtrate was concentrated, and the crude product was purified by silica gel column chromatography eluted with 0-50% ethyl acetate in hexanes to afford an off-white solid (0.22 g, 56%). ¹H NMR (400 MHz, CDCl₃) δ: 8.66 (d, 1H), 7.81-7.77 (m, 2H), 7.56 (d, 1H), 7.41 (dd, 1H), 2.77 (s, 3H).
Step 3

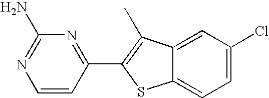

### 4-(5-Chloro-3-methylbenzo[b]thiophen-2-yl)pyrimidin-2-amine

To a solution of 2-chloro-4-(5-chloro-3-methylbenzo[b]thiophen-2-yl)pyrimidine in EtOH (3.4 mL), was added NH₄OH (0.26 mL of 28% in water) in a pressure tube. The reaction vessel was sealed and heated to 80° C. overnight. The reaction mixture was extracted three times with ethyl acetate (100 mL), washed with water, brine, dried over Na₂SO₄, and filtered. The filtrate was concentrated and purified by silica gel column chromatography eluted with 0-50% ethyl acetate in hexanes to afford an off-white solid (0.085 g, 45%). ¹H NMR (400 MHz, DMSO-d₆) δ: 8.35 (d, 1H), 8.01-7.97 (m, 2H), 7.45 (dd, 1H), 6.98 (d, 1H), 6.80 (s, br, 2H), 2.65 (s, 3H); LCMS: (M+1)⁺: 278.93.

### EXAMPLE 2

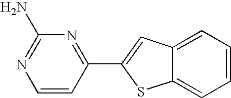

### 4-(Benzo[b]thiophen-2-yl)pyrimidin-2-amine

The title compound was prepared analogously to 4-(5-chloro-3-methylbenzo[b]thiophen-2-yl)pyrimidin-2-amine as described in Example 1, where benzo[b]thiophen-2-ylboronic acid was substituted for 5-chlorobenzo[b]thiophen-2-ylboronic acid in step 1 of that sequence. ¹H NMR (400 MHz, DMSO-d₆) δ: 8.37 (s, 1H), 8.34 (d, 1H), 8.02-8.00 (m, 1H), 7.93-7.91 (m, 1), 7.47-7.40 (m, 2H), 7.35 (d, 1H): LCMS: (M+1)⁺: 227.83.

### EXAMPLE 3

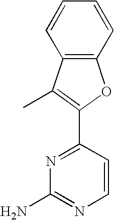

### 4-(3-Methylbenzofuran-2-yl)pyrimidin-2-amine

Step 1

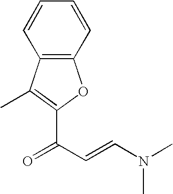

### (E)-3-(Dimethylamino)-1-(3-methylbenzofuran-2-yl)prop-2-en-1-one

A 20 mL screw cap vial was charged with 1-(3-methylbenzofuran-2-yl)ethanone (174 mg, 1.00 mmol), and N,N-dimethylformamide dimethyl acetal (3 mL), then placed in a 100° C. oil bath and stirred for 16 h and then evaporated. The crude product was purified by silica gel chromatography, eluting with EtOAc in hexanes, giving the product as a pale yellow solid (161 mg, 70%.) LCMS (M+1⁺): 230.09.
Step 2

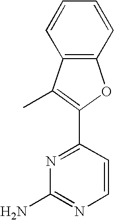

### 4-(3-Methylbenzofuran-2-yl)pyrimidin-2-amine

A 20 mL screw cap vial was charged with (E)-3-(dimethylamino)-1-(3-methylbenzofuran-2-yl)prop-2-en-1-one (153 mg, 0.667 mmol), guanidine hydrochloride (191 mg, 2.00 mmol), K₂CO₃ (277 mg, 2.00 mmol), and 2-methoxyethanol (3.3 mL), then placed in a 130° C. oil bath and stirred for 1.5 h. The reaction was concentrated, slurried in H₂O (10 mL), and the resulting solid material was collected by filtration and washed with H₂O (10 mL). The filter cake was dissolved in methanol, filtered and evaporated to give the product as an off-white solid (125 mg, 83%). ¹H NMR (400 MHz, DMSO-d₆) δ: 8.34 (d, 1H), 7.71 (m, 1H), 7.60 (m, 1H), 7.41 (m, 1H), 7.31 (m, 1H), 7.00 (d, 1H), 6.72 (bs, 2H), 2.70 (s, 3H). LCMS (M+1⁺): 226.18.

### EXAMPLE 4

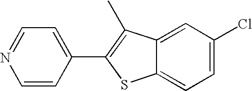

### 4-(5-Chloro-3-methylbenzo[b]thiophen-2-yl)pyridine

The title compound was prepared analogously to 4-(5-chloro-3-methylbenzo[b]thiophen-2-yl)pyrimidin-2-amine as described in Example 1, where 4-bromopyridine was substituted for 2,4-dichloropyrimidine in step 2 of that sequence. ¹H NMR (400 MHz, CDCl₃) δ: 8.71 (d, 2H), 7.77-7.73 (m, 2H), 7.45-7.43 (m, 2H), 7.35 (dd, 1H), 2.49 (s, 3H): LCMS: (M+1)⁺: 259.38.

### EXAMPLE 5

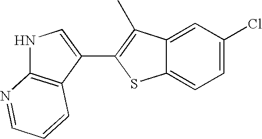

### 3-(5-Chloro-3-methylbenzo[b]thiophen-2-yl)-1H-pyrrolo[2,3-b]pyridine

The title compound was prepared analogously to 4-(5-chloro-3-methylbenzo[b]thiophen-2-yl)pyrimidin-2-amine as described in Example 1, where 3-bromo-1H-pyrrolo[2,3-b]pyridine (prepared as described in J. Am. Chem. Soc. 1956, 78, 1247 by R. Robinson et. al.) was substituted for 2,4-dichloropyrimidine in step 2 of that sequence. ¹H NMR (400 MHz, DMSO-d₆) δ: 12.22 (s, 1H), 8.31 (d, 1H), 8.12 (d, 2H), 7.99 (d, 1H), 7.85 (s, 2H), 7.40 (d, 1H), 7.2-7.15 (m, 1H), 2.41 (s, 3H): LCMS: (M+1)⁺: 300.63.

### EXAMPLE 6

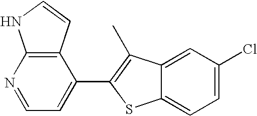

### 4-(5-Chloro-3-methylbenzo[b]thiophen-2-yl)-1H-pyrrolo[2,3-b]pyridine

The title compound was prepared analogously to 4-(5-chloro-3-methylbenzo[b]thiophen-2-yl)pyrimidin-2-amine as described in Example 1, where 4-bromo-1H-pyrrolo[2,3-b]pyridine (prepared as described in Org. Lett. 2003, 5, 5023-5025) was substituted for 2,4-dichloropyrimidine in step 2 of that sequence. ¹H NMR (400 MHz, DMSO-d₆) δ: 11.96 (s, 1H), 8.33 (d, 1H), 8.06 (d, 2H), 7.94 (d, 1H), 7.60-7.59 (m, 1H), 7.46 (dd, 1H), 7.19 (d, 1H), 6.51-6.50 (m, 1H), 2.40 (s, 3H); LCMS: (M+1)⁺: 300.64.

### EXAMPLE 7

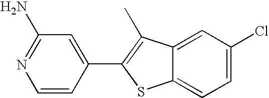

### 4-(5-Chloro-3-methylbenzo[b]thiophen-2-yl)pyridin-2-amine

Step 1

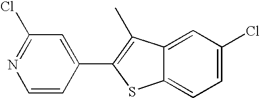

### 2-Chloro-4-(5-chloro-3-methylbenzo[b]thiophen-2-yl)pyridine

To a solution of 5-chloro-3-methylbenzo[b]thiophen-2-ylboronic acid (0.3 g, 1.3 mmol) and 2-chloro-4-iodopyridine (0.32 g, 1.3 mmol) in 3:1 THF/water, was added aqueous solution of Na₂CO₃ (1.6 mL, 2M). The mixture was degassed three times, back filled with nitrogen, and Pd(Ph₃P)₂Cl₂ (0.091 g 0.13 mmol) was added in one portion. The reaction mixture was stirred and heated to 70° C. for 2 hours, until LCMS confirmed the completion of the reaction. The reaction mixture was extracted three times with ethyl acetate (100 mL), washed with water, brine, dried over Na₂SO₄, and filtered. The filtrate was concentrated in vacuo to give the crude product that was purified by silica gel column chromatography eluted with 0-50% ethyl acetate in hexanes to afford a yellow solid (0.31 g, 79% yield). LCMS: (M+1)⁺: 293.76.
Step 2

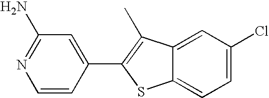

### 4-(5-Chloro-3-methylbenzo[b]thiophen-2-yl)pyridin-2-amine

To a solution of 2-chloro-4-(5-chloro-3-methylbenzo[b]thiophen-2-yl)pyridine (0.05 g, 0.17 mmol) in THF, was added Pd₂(dba)₃ (4.9 mg, 0.009 mmol), and biphenyl-2-yldicyclohexylphosphine (7.1 mg, 0.02 mmol). The reaction mixture was degassed three times and back filled with nitrogen. LHMDS (0.22 mmol, 0.22 mL of 1M THF solution) was added in one portion. The mixture was stirred and heated to 65° C. for 4 hours. The reaction mixture was cooled down, and diluted with water. It was extracted three times with ethyl acetate (25 mL), washed with water, brine, dried over Na₂SO₄, and filtered. The filtrate was concentrated and purified by reversed phase C-18 column chromatography eluted with 30-100% acetonitrile in water in the presence of 0.1% TFA affording an off-white solid (0.006 g, 13% yield). LCMS: (M+1)⁺: 274.87.

### EXAMPLE 8

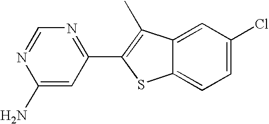

### 6-(5-Chloro-3-methylbenzo[b]thiophen-2-yl)pyrimidin-4-amine

The title compound was prepared analogously to 4-(5-chloro-3-methylbenzo[b]thiophen-2-yl)pyrimidin-2-amine as described in Example 7, where 4,6-dichloropyrimidine was substituted for 2,4-dichloropyrimidine in step 2 of that sequence. ¹H NMR (400 MHz, DMSO-d₆) δ: 8.50 (s, 1H), 8.03 (d, 1H), 7.98 (d, 1H), 7.59 (s, 2H), 7.47 (dd, 1H), 6.90 (s, 1H), 2.62 (s, 3H): LCMS: (M+11): 278.02.

### EXAMPLE 9

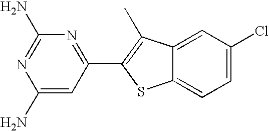

### 6-(5-Chloro-3-methylbenzo[b]thiophen-2-yl)pyrimidine-2,4-diamine

The title compound was prepared analogously to 4-(5-chloro-3-methylbenzo[b]thiophen-2-yl)pyrimidin-2-amine as described in Example 7, where 6-chloropyrimidine-2,4-diamine was substituted for 2,4-dichloropyrimidine in step 2 of that sequence. LCMS: (M+1)⁺: 291.09.

### EXAMPLE 10

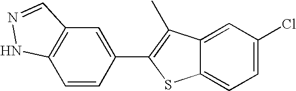

### 5-(5-Chloro-3-methylbenzo[b]thiophen-2-yl)-1H-indazole

The title compound was prepared analogously to 4-(5-chloro-3-methylbenzo[b]thiophen-2-yl)pyrimidin-2-amine as described in Example 7, where 5-bromo-1H-indazole was substituted for 2,4-dichloropyrimidine in step 2 of that sequence. ¹H NMR (400 MHz, DMSO-d₆) δ: 8.17 (s, 1H), 7.99 (d, 1H), 7.95 (s, 1H), 7.86 (d, 2H), 7.67 (d, 1H), 7.53 (dd, 1H), 7.40 (dd, 1H), 2.42 (s, 3H): LCMS: (M+1)⁺: 298.96.

### EXAMPLE 11

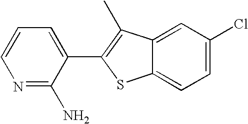

### 3-(5-Chloro-3-methylbenzo[b]thiophen-2-yl)pyridin-2-amine

The title compound was prepared analogously to 4-(5-chloro-3-methylbenzo[b]thiophen-2-yl)pyrimidin-2-amine as described in Example 7, where 3-bromopyridin-2-amine was substituted for 2,4-dichloropyrimidine in step 2 of that sequence. ¹H NMR (400 MHz, CD₃OD) δ: 8.04-7.99 (m, 2H), 7.89 (d, 1H), 7.86 (d, 1H), 7.43 (dd, 1H), 7.05 (dd, 1H), 2.29 (s, 3H): LCMS: (M+1)⁺: 275.01

### EXAMPLE 12

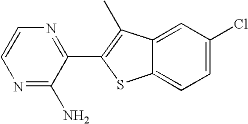

### 3-(5-Chloro-3-methylbenzo[b]thiophen-2-yl)pyrazin-2-amine

The title compound was prepared analogously to 4-(5-chloro-3-methylbenzo[b]thiophen-2-yl)pyrimidin-2-amine as described in Example 7, where 3-bromopyrazin-2-amine was substituted for 2,4-dichloropyrimidine in step 2 of that sequence. ¹H NMR (400 MHz, CD₃OD) δ: 8.04 (d, 2H), 7.92 (d, 1H), 7.88 (d, 1H), 7.84 (d, 1H), 7.40 (dd, 1H), 2.32 (s, 3H): LCMS: (M+11): 275.99.

### EXAMPLE 13

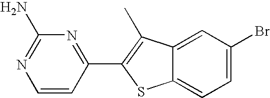

### 4-(5-Bromo-3-methylbenzo[b]thiophen-2-yl)pyrimidin-2-amine

Step 1

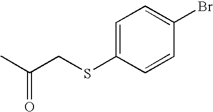

### 1-(4-Bromophenylthio)propan-2-one

A 500 mL round bottom flask was charged with a solution of 4-bromobenzenethiol (9 g, 47.62 mmol), pyridine (20 g, 253.16 mmol), in Et₂O (80 mL). To the reaction mixture 1-bromopropan-2-one (6.9 g, 51.49 mmol) was added in several batches, and the resulting solution was allowed to stir at room temperature. The mixture was then filtered, and the filtered solid was washed twice with 0.2N hydrochloric acid (100 mL). The filtrate was dried over MgSO₄, concentrated, and purified by silica gel column chromatography eluted with 10:1 petroleum ether/ethyl acetate to afford the product in 10 g (80% yield) as a white solid.
Step 2

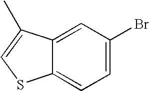

### 5-Bromo-3-methylbenzo[b]thiophene

A 500 mL round bottom flask was charged with 1-(4-bromophenylthio)propan-2-one (12.2 g, 49.80 mmol), in aqueous H₂SO₄ (250 mL). The resulting solution was heated to 110° C. for 10 hours. Work up: the reaction mixture was extracted three times with methylene chloride (100 mL), washed with Na₂CO₃ (20% aqueous solution), dried over Na₂SO₄, and concentrated. The crude product was purified by silica gel column chromatography eluted with 10:1 petroleum ether/ethyl acetate to afford the product in 8 g (42% yield) as a yellow oil.
Step 3

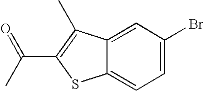

### 1-(5-Bromo-3-methylbenzo[b]thiophen-2-yl)ethanone

A 250 mL round bottom flask was charged with a solution of 5-bromo-3-methylbenzo[b]thiophene (5 g, 21.81 mmol) in CS₂ (10 mL). To this mixture was added AlCl₃ (5.9 g, 43.79 mmol) followed by addition of acetyl chloride (2.1 g, 26.48 mmol) dropwise at 0° C. The resulting solution was stirred, and allowed to warm to room temperature for 3 hours. The reaction was quenched by addition of water/ice (20 mL), and the pH was adjusted to 4 by the addition of hydrochloric acid (5% aqueous solution). The resulting mixture was extracted three times with ethyl acetate (30 mL), dried over MgSO₄, and concentrated. The crude product was purified by silica gel column chromatography eluted with 10:1 petroleum ether/ethyl acetate to afford title compound in 3.5 g (51% yield) as a white solid.
Step 4

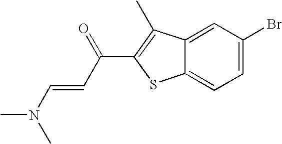

### (E)-1-(5-Bromo-3-methylbenzo[b]thiophen-2-yl)-3-(dimethylamino)prop-2-en-1-one

A 100 mL round bottom flask was charged with (5-bromo-3-methylbenzo[b]thiophen-2-yl)ethanone (3.5 g, 13.11 mmol), and DMFDMA (10 mL). The resulting solution was heated to 80° C. overnight. The residue was concentrated to afford 2.5 g (59% yield) of the product as a yellow solid. The product was used in the next step without further purification
Step 5

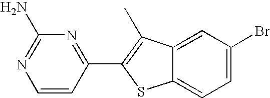

### 4-(5-Bromo-3-methylbenzo[b]thiophen-2-yl)pyrimidin-2-amine

A 100 mL round bottom flask was charged with a solution of (E)-1-(5-bromo-3-methylbenzo[b]thiophen-2-yl)-3-(dimethylamino)prop-2-en-1-one (1.5 g, 4.66 mmol), sodium ethoxide (1.8 g, 26.47 mmol), and guanidine hydrochloride (1.5 g, 15.71 mmol) and EtOH (50 mL). The resulting mixture was refluxed for 36 hours. The mixture was filtered, and the filtrate was concentrated to afford 0.8 g (54% yield) of the title compound as a yellow powder. ¹H NMR (300 MHz, CDCl₃) δ: 8.36 (d, 1H), 8.10 (s, 1H), 7.95 (d, 1H), 7.57 (d, 1H), 6.98 (d, 1H), 6.79 (s, 2H), 2.66 (s, 3H). LCMS: (M+1)⁺: 321.00.

### EXAMPLE 14

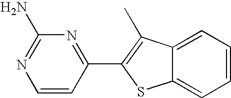

### 4-(3-Methylbenzo[b]thiophen-2-yl)pyrimidin-2-amine

A 10 mL round bottom flask was charged with 4-(5-bromo-3-methylbenzo[b]thiophen-2-yl)pyrimidin-2-amine (0.05 g, 0.15 mmol) prepared as described in Example 13, and THF (0.8 mL), then cooled to −78° C. To the resulting mixture was added dropwise n-butyl lithium (0.39 mmol, 0.24 mL of 1.6 M solution in hexanes) at −78° C. over 15 min. Work up: the reaction was quenched with methanol at −78° C., warmed to room temperature, and concentrated. The crude material was purified by C18 reverse phase semi-preparative HPLC, eluted with 10-100% acetonitrile in water (0.1% TFA), affording 0.02 g (53% yield) as a pale yellow solid. ¹H NMR (400 MHz, DMSO-d₆) δ: 8.35 (d, 1H), 7.99-7.91 (m, 2H), 7.46-7.44 (m, 2H), 7.06 (d, 1H), 2.70 (s, 3H). LCMS: (M+1)⁺: 242.03.

### EXAMPLE 15

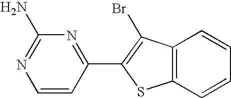

### 4-(3-Bromobenzo[b]thiophen-2-yl)pyrimidin-2-amine

Step 1:

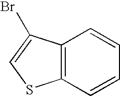

### 3-Bromobenzo[b]thiophene

A 2 L round bottom flask was charged with benzo[b]thiophene (50 g, 373.13 mmol), CH₂Cl₂ (800 mL), and NaOAc (62 g, 756.10 mmol). To this was added a solution of Br₂ (34 g, 212.50 mmol) and CH₂Cl₂ (700 mL), dropwise at 0° C. over 3 hours. The resulting solution was stirred for 1 hour while the temperature was maintained at 0° C. Reaction progress was monitored by TLC (EtOAc/petroleum ether=1:100). Work up: the resulting mixture was washed three times with saturated NaHSO₃(200 mL). The organic layers were combined, dried over MgSO₄, concentrated, and purified by flash chromatography with a 1:1000 EtOAc/petroleum ether. This resulted in 70 g (88%) of product as a colorless oil.
Step 2

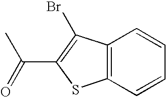

### (E)-3-(Dimethylamino)-1-(3-methyl-5-phenoxybenzo[b]thiophen-2-yl)prop-2-en-1-one

A 1000 mL round bottom flask was charged with 3-bromobenzo[b]thiophene (30 g, 141.51 mmol), and CS₂ (500 mL). To this solution was added AlCl₃ (37.6 g, 284.85 mmol) in several batches. To the above was added acetyl chloride (11.2 g, 143.59 mmol) dropwise with stirring at 0° C. The resulting solution was stirred for 1.5 hours while the temperature was maintained at 0° C. in an ice bath. Reaction progress was monitored by TLC (EtOAc/petroleum ether=1:5). Work up: the reaction mixture was then quenched by the adding 1000 g of H₂O/ice and stirring for 10 min. The aqueous layer was extracted three times with of CH₂Cl₂ (300 mL). The combined organic layers were washed three times with brine (200 mL), dried over MgSO₄, and concentrated, giving 22 g (62%), of the product as a light yellow solid.
Step 3

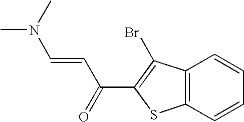

### 4-(3-Methyl-5-phenoxybenzo[b]thiophen-2-yl)pyrimidin-2-amine

A 500 mL round bottom flask was charged with 1-(3-bromobenzo[b]thiophen-2-yl)ethanone (20 g, 78.74 mmol), and DMFDMA (200 mL). The resulting solution was stirred for 15 hours at reflux. Reaction progress was monitored by TLC (EtOAc/petroleum ether=10:1). Work up: the reaction mixture was cooled at which point a solid formed. The solid was filtered, and washed three times with hexanes (100 mL). This resulted in 20 g of product as a yellow solid, that was used directly without further purification.
Step 4

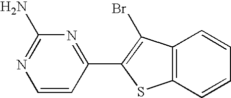

### 4-(3-Bromobenzo[b]thiophen-2-yl)pyrimidin-2-amine

A 500 mL round bottom flask was charged with 1-(3-bromobenzo[b]thiophen-2-yl)-3-(dimethylamino)prop-2-en-1-one (20 g, 64.72 mmol), ethanol (300 mL), and guanidine (9.5 g, 161.02 mmol). The resulting solution was stirred for 1 hour at reflux. Reaction progress was monitored by TLC (EtOAc/petroleum ether=1:1). Work up: half of solvent was removed by evaporation giving slurry. Solid was isolated by filtration, then washed three times with 80 mL of cold ethanol, giving 20.5 g (94.6%) of the title compound. ¹H NMR (300 MHz, DMSO-d₆) δ: 8.45 (d, 1H), 8.09 (d, 1H), 7.88 (m, 1H), 7.64 (m, 1H), 7.60-7.54 (m, 2H), 6.90 (s, 2H). LCMS (M+1)⁺: 306.10.

### EXAMPLE 16

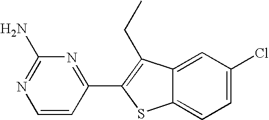

### 4-(5-Chloro-3-ethylbenzo[b]thiophen-2-yl)pyrimidin-2-amine

Step 1

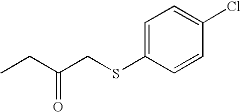

### 1-(4-Chlorophenylthio)butan-2-one

A 100 mL round bottom flask was charged with 4-chlorobenzenethiol (7 g, 48.28 mmol), K₂CO₃ (115 g, 833.33 mmol) and DMF (80 mL). To the reaction mixture 1-bromobutan-2-one (7.4 g, 49.01 mmol) was added dropwise at 0° C. The resulting solution was stirred at room temperature for 2 hours. Work up: the reaction mixture was diluted with ethyl acetate (200 mL), washed three times with water (400 mL), dried over MgSO₄, filtered, and concentrated. The crude product was purified by silica gel chromatography eluted with EtOAc/PE (1/30) affording the title compound in 5 g (48% yield) as a colorless oil.
Step 2

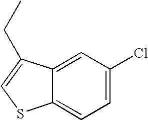

### 5-Chloro-3-ethylbenzo[b]thiophene

A 500 mL 3-necked round bottom flask was charged with polyphosphoric acid (50 g), in 1-chlorobenzene (300 mL). To this was added 1-(4-chlorophenylthio)butan-2-one (21 g, 97.67 mmol) dropwise while refluxing. The resulting solution was refluxed overnight. The reaction was cooled, and the pH adjusted to 7 by addition of KOH (50% aqueous solution). The mixture was extracted three times with EtOAc (300 mL), dried over MgSO₄, filtered, and concentrated. The crude product was purified by silica gel column eluted with EtOAc/PE (1/100) resulting in 17 g (89% yield) of the title compound as a white solid.
Step 3

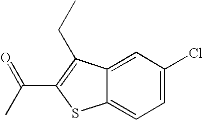

### 1-(5-Chloro-3-ethylbenzo[b]thiophen-2-yl)ethanone

A 500 mL 3-necked round bottom flask was charged with 5-chloro-3-ethylbenzo[b]thiophene (8.5 g, 10.26 mmol), and acetyl chloride (800 mg, 10.26 mmol) in CS₂ (125 mL). To this mixture was added AlCl₃ (1.4 g, 10.37 mmol) in several batches at 0° C. The resulting solution was allowed stir at 0° C. overnight. Work up: the reaction was poured over 200 g of ice water, extracted three times with methylene chloride (50 mL), washed with brine, dried over MgSO₄, and concentrated. The crude product was purified by silica gel column chromatography eluted with a 1:10 EtOAc/PE. The title compound was obtained in 1 g (41% yield) a white solid.
Step 4

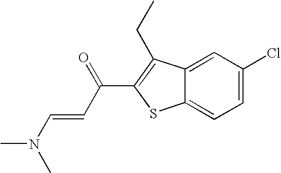

### (E)-1-(5-Chloro-3-ethylbenzo[b]thiophen-2-yl)-3-(dimethylamino)prop-2-en-1-one

A 100 mL round bottom flask was charged with 1-(5-chloro-3-ethylbenzo[b]thiophen-2-yl)ethanone (1 g, 4.20 mmol) and DMFDMA (10 mL) at room temperature. The resulting solution was refluxed for 1 hour. Work-up: the mixture was diluted with EtOAc (50 mL), washed three times with water (50 mL), brine (50 mL), and dried over Na₂SO₄. The reaction afforded 1.1 g (92% yield) of the title compound as a yellow solid. The product was used in the next step without further purification.
Step 5

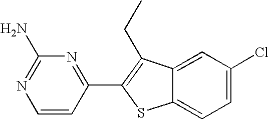

### 4-(5-Chloro-3-ethylbenzo[b]thiophen-2-yl)pyrimidin-2-amine

A 100 mL round bottom flask was charged with ethanol (20 mL). To this was added Na (150 mg, 6.52 mmol) at room temperature in small portions, followed by addition of guanidine hydrochloride (450 mg, 4.74 mmol). To the resulting mixture (E)-1-(5-chloro-3-ethylbenzo[b]thiophen-2-yl)-3-(dimethylamino)prop-2-en-1-one (1.2 g, 4.10 mmol) in ethanol (40 mL) was added dropwise. The reaction mixture was heated to reflux for 3 hours. Work up: the mixture was concentrated, neutralized, and purified by recrystallization from ethanol to afford 1 g (84% yield) of the title compound as a white solid. ¹H NMR (300 MHz, DMSO-d₆) δ: 8.37 (d, 1H), 8.01 (dd, 2H), 7.46 (s, 1H), 6.92 (d, 1H), 6.82 (s, 2H), 3.17 (q, 2H), 1.26 (t, 3H): LCMS (M+H)⁺: 290

### EXAMPLE 17

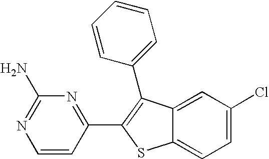

### 4-(5-Chloro-3-phenylbenzo[b]thiophen-2-yl)pyrimidin-2-amine

Step 1

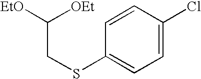

### (4-Chlorophenyl)(2,2-diethoxyethyl)sulfane

A 3 L round bottom flask was charged with 4-chlorobenzenethiol (72.5 g, 500 mol), K₂CO₃ (138 g, 1.00 mol), and DMF (0.5 L). To this mixture was added a solution of 2-bromo-1,1-diethoxyethane (138 g, 0.60 mol) in DMF (250 mL) dropwise at 0° C., over 3 hours. The reaction was stirred at 0° C. for 2 h. Work-up: the mixture was diluted with EtOAc (750 mL), washed three times with water (500 mL), and dried over MgSO₄. The crude product was distilled (66-68° C., at 17 mm Hg) to remove the excess 2-bromo-1,1-diethoxyethane. The remaining residue was purified by silica gel column chromatography eluted with 1:60 EtOAc/PE affording 90 g (55% yield) of the title compound as pale yellow oil. ¹H-NMR (300 MHz, CDCl₃): δ:: 7.24-7.35 (m, 4H) 4.63-4.69 (m, 1H), 3.50-3.75 (m, 4H), 3.12 (d, 2H), 1.19-1.28 (m, 6H).
Step 2

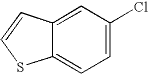

### 5-Chlorobenzo[b]thiophene

A 25 mL round bottom flask was charged with (4-chlorophenyl)(2,2-diethoxyethyl)sulfane (500 mg, 1.92 mmol) and chlorobenzene (2 mL). The resulting mixture was added dropwise into boiling polyphosphoric acid (1 g) in chlorobenzene (5 mL) over 5 min. Work-up: the mixture was poured over ice water (25 mL), extracted three times with EtOAc (25 mL), washed with brine (50 mL), and dried over Na₂SO₄. The mixture was concentrated, and purified by SiO₂ flash chromatography eluting with PE to afford the title compound in 290 mg (90% yield), as an off white solid.
Step 3

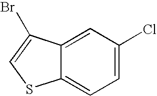

### 3-Bromo-5-chlorobenzo[b]thiophene

A solution of Br₂ (160 mg, 1.00 mmol) in methylene chloride (5 mL) was added dropwise to a 25 mL round bottom flask charged with 5-chlorobenzo[b]thiophene (169 mg, 1.00 mmol), and NaOAc (164 mg, 2.00 mmol) in methylene chloride (10 mL) at 0° C. over 5 min. The resulting mixture was added dropwise into boiling polyphosphoric acid (1 g) in chlorobenzene (5 mL) over 5 min. Work-up: the mixture was poured into 10% aqueous solution of NaHSO₃ (20 mL), extracted three times with EtOAc (20 mL), and dried over MgSO₄. The mixture was concentrated to give the title compound in 0.247 g (99% yield) as a pale yellow solid (mp 84° C.). ¹H-NMR (300 MHz, DMSO-d₆): δ:: 7.45-7.56 (m, 1H), 7.76-7.77 (d, 1H), 7.99-8.18 (m, 2H).
Step 4

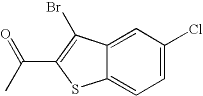

### 1-(3-Bromo-5-chlorobenzo[b]thiophen-2-yl)ethanone

A 25 mL round bottom flask was charged with 3-bromo-5-chlorobenzo[b]thiophene (148 mg, 0.60 mmol) and CS₂ (5 mL). To the resulting mixture, AlCl₃ (0.153 g, 0.60 mmol) was added, followed by dropwise addition (10 min.) of acetyl chloride (55 mg, 0.70 mmol) in CS₂ (1 mL) at 0° C. The resulting solution was stirred at this 0° C. for 3 hours. Work-up: the mixture was washed with water (5 mL) and the pH was adjusted to 4 by the addition of hydrochloric acid (10% aqueous solution). The resulting mixture was extracted three times with EtOAc (10 mL), and dried over MgSO₄. The mixture was concentrated to give the title compound in 0.17 g (98% yield) as a pale yellow solid. ¹H-NMR (300 MHz, DMSO-d₆): δ:: 8.17-8.20 (d, 1H), 7.93-7.94 (d, 1H), 7.66-7.70 (dd, 1H), 2.78 (s, 3H).
Step 5

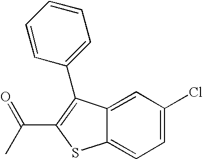

### 1-(5-Chloro-3-phenylbenzo[b]thiophen-2-yl)ethanone

A 50 mL round bottom flask purged with nitrogen was charged with 1-(3-bromo-5-chlorobenzo[b]thiophen-2-yl)ethanone (1.2 g, 4.14 mmol), K₂CO₃ (1.72 g, 12.45 mmol), phenylboronic acid (600 mg, 4.92 mmol), EtOH (5 mL), Pd[(PPh₃)]₄ (600 mg, 0.52 mmol), and toluene (20 mL). The mixture was refluxed for 4 hours. Work-up: the mixture was washed with water (5 mL), the pH was adjusted to 7 by the addition of hydrochloric acid (1 M aqueous solution, 10 mL), extracted three times with EtOAc (10 mL), and dried over MgSO₄. The crude material was concentrated and purified by silica gel column chromatography eluted with EtOAc/PE (1/25) affording 0.68 g (57% yield) of the title compound as a white solid.
Step 6

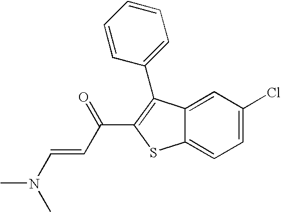

### (E)-1-(5-Chloro-3-phenylbenzo[b]thiophen-2-yl)-3-(dimethylamino)prop-2-en-1-one

A 25 mL round bottom flask was charged with 1-(5-chloro-3-phenylbenzo[b]thiophen-2-yl)ethanone (240 mg, 0.84 mmol) and DMFDMA (6 mL) at room temperature. The resulting solution was refluxed for 12 h. Work-up: the mixture was diluted with EtOAc (10 mL), washed three times with water (50 mL), dried over MgSO₄, and concentrated affording 0.25 g (87% yield), as a yellow solid. The crude product was used in the next step without further purification.
Step 7

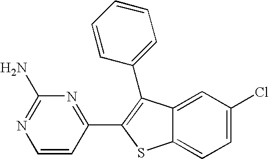

### 4-(5-Chloro-3-phenylbenzo[b]thiophen-2-yl)pyrimidin-2-amine

Guanidine hydrochloride (2.09 g, 21.88 mmol) was added to a 100 mL round bottom flask charged with a freshly prepared solution of EtONa (21.91 mmol) in ethanol (50 mL) at room temperature. The resulting solution was refluxed for 0.5 hours. The solution was cooled and filtered to remove sodium chloride. To the filtrate was added (E)-1-(5-chloro-3-phenylbenzo[b]thiophen-2-yl)-3-(dimethylamino) prop-2-en-1-one (2.5 g, 7.31 mmol). The resulting solution was refluxed for 4 hours, then cooled, and filtered. The filtered solid was washed three times with cold ethanol (10 mL) affording 1.9 g (80% yield) of the title compound as a pale yellow solid. ¹H-NMR (300 MHz, DMSO-d₆): δ: 8.13 (d, 1H), 8.02 (d, 1H), 7.42-7.61 (m, 6H), 7.23 (d, 1H), 6.84 (s, 2H), 5.96 (d, 1H): LCMS (M+H)⁺: 338.

### EXAMPLE 18

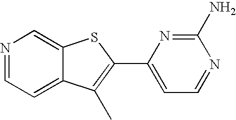

### 4-(3-Methylthieno[2,3-c]pyridin-2-yl)pyrimidin-2-amine

Step 1

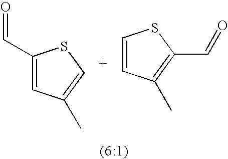

### 4-Methylthiophene-2-carbaldehyde

A 1000 mL round bottom flask under nitrogen was charged with ether (500 mL, anhydrous), and nBuLi (163 mL, 325 mmol), then cooled to 0° C., where 3-methylthiophene (28.4 mL, 295 mmol) was added dropwise over 15 min. This solution was stirred for 2 hr at room temperature. To the anion was added dropwise a solution of DMF (30 mL, 384 mmol) dissolved in ether (100 mL, anhydrous). The resulting solution was stirred overnight at room temperature. Reaction progress was monitored by TLC (20% ethyl acetate/hexanes). Work-up: the mixture was poured onto ice, washed with HCl (1N aq.), NaHCO₃ (1N aq.), brine, dried with MgSO₄, concentrated, and distilled under high vacuum. The product was collected at 92° C., had a mass of 30.6 g, 82% yield. It contained 17% of the 3-methyl isomer as indicated by NMR. ¹H NMR (400 MHz, CDCl₃) δ 9.87 (s, 1H), 7.58 (s, 1H), 7.37 (s, 1H), 2.33 (s, 3H).
Step 2

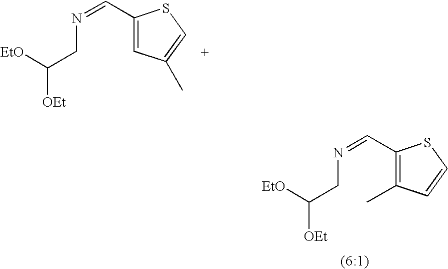

### 2,2-Diethoxy-N-((4-methylthiophen-2-yl)methylene)ethanamine

A 100 mL round bottom flask equipped with Dean-Stark trap was charged with 4-methylthiophene-2-carbaldehyde (5.93 mL, 55 mmol), 2,2-diethoxyethanamine (6.31 g, 50 mmol), and toluene (30 mL). The resulting solution was refluxed overnight, at which time the theoretical amount of water had been collected. The reaction was concentrated under vacuum to an oil, which was used in the following step without further purification.
Step 3

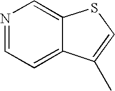

### 3-Methylthieno[2,3-c]pyridine

A 500 mL round bottom flask was charged with polyphosphoric acid (216 g), heated to 120° C., where 2,2-diethoxy-N-((4-methylthiophen-2-yl)methylene)ethanamine (55 mmol crude from previous step) was added slowly over 15 min, while vigorously stirred. The resulting black mixture was stirred for an additional 20 min. at this temperature. Reaction progress was monitored by TLC (40% ethyl acetate/hexanes, Rf=0.4). Work-up: the mixture was poured onto ice (exothermic), and extracted with ether (2×200 mL) which was discarded. The remaining aqueous solution was carefully made basic (very exothermic) with a syrup of concentrated NaOH/water while being cooled in an ice bath. The resulting solution was extracted with ether (4×500 mL), dried with MgSO₄, filtered, concentrated, and purified by flash chromatography (30 to 80% ethyl acetate/hexanes, gradient elution). This resulted in a brown oil that solidified after drying overnight under high vacuum (1.95 g, 18% yield for two steps). ¹H NMR (400 MHz, CDCl₃) δ 9.12 (s, 1H), 8.53 (d, 1H), 7.61 (d, 1H), 7.33 (s, 1H), 2.46 (s, 3H). LCMS (M+1)⁺: 150.11.
Step 4

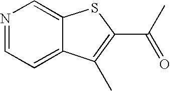

### 1-(3-Methylthieno[2,3-c]pyridin-2-yl)ethanone

A 50 mL round bottom flask under nitrogen atmosphere was charged with diisopropylamine (1.90 mL, 13.4 mmol), THF (27 mL, anhydrous), cooled to 0° C., and treated with n-butyl lithium (8.4 mL, 13.4 mmol). After 10 min at this temperature, 3-methylthieno[2,3-c]pyridine (1.00 g, 6.7 mmol) dissolved in THF (7 mL, anhydrous) was added in one portion. The resulting dark green/yellow solution was stirred for 1 hour, then treated with N-methoxy-N-methylacetamide (1.38 g, 13.4 mmol) and stirred for an additional 2 hours at room temperature. Reaction progress was monitored by TLC (40% ethyl acetate hexanes, Rf=0.2). Work-up: the reaction mixture was quenched with NH₄Cl (1N aqueous), extracted with ether (2×100 mL), dried with MgSO₄, filtered, and concentrated to a slurry. The solid from the slurry was isolated by filtration, rinsed with ether, and dried under high vacuum, giving the product as tan solid (0.60 g, 47% yield). ¹H NMR (400 MHz, CDCl₃) δ 9.18 (s, 1H), 8.61 (d, 1H), 7.73 (d, 1H), 2.75 (s, 3H), 2.69 (s, 3H). LCMS (M+1)⁺: 192.12.
Step 5

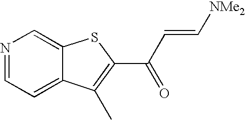

### (E)-3-(Dimethylamino)-1-(3-methylthieno[2,3-c]pyridin-2-yl)prop-2-en-1-one

A 10 mL round bottom flask was charged with of 1-(3-methylthieno[2,3-c]pyridin-2-yl)ethanone (191 mg, 1.0 mmol), and dimethylformamide dimethyl acetal (3 mL). The resulting solution was stirred overnight in an 80° C. oil bath. Reaction progress was monitored by LCMS. Work-up: the reaction was cooled to room temperature where a solid formed, then diluted with ether and sonicated giving a slurry. The solid was isolated by filtration, then rinsed with ether, and dried under high vacuum, giving the product as a bright yellow solid (218 mg, 89% yield). ¹H NMR (400 MHz, CDCl₃) δ 9.10 (s, 1H), 8.55 (d, 1H), 7.80 (d, 1H), 7.65 (d, 1H), 5.62 (d, 1H), 3.19 (s, 1H), 2.96 (s, 1H), 2.71 (s, 3H).
Step 6

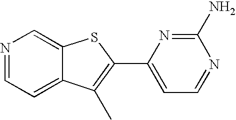

### 4-(3-Methylthieno[2,3-c]pyridin-2-yl)pyrimidin-2-amine

A 10 mL round bottom flask was charged with (E)-3-(dimethylamino)-1-(3-methylthieno[2,3-c]pyridin-2-yl)prop-2-en-1-one (123 mg, 0.5 mmol), guanidine-HCl (143 mg, 1.5 mmol), K₂CO₃ (207 mg, 1.5 mmol), and 2-methoxyethanol (2.0 mL). The resulting mixture was heated in a 130° C. oil bath for 1.5 hr. Reaction progress was monitored by LCMS. Work-up: the reaction was concentrated, diluted with water, extracted with 2% methanol/methylene chloride (3×30 mL), dried with MgSO₄, filtered, and concentrated to a slurry. Solid was isolated by filtration, rinsed with methlyene chloride, and dried under high vacuum, giving the title compound as a light yellow powder (76 mg, 63% yield). ¹H NMR (400 MHz, DMSO-d₆) δ 9.23 (s, 1H), 8.52 (d, 1H), 8.39 (d, 1H), 7.86 (d, 1H), 7.05 (d, 1H), 6.87 (s, 2H), 2.66 (s, 3H). LCMS (M+1)⁺: 243.09.

### EXAMPLE 19

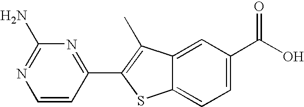

### 2-(2-Aminopyrimidin-4-yl)-3-methylbenzo[b]thiophene-5-carboxylic acid

A 50 mL 3-necked round bottom flask purged and back filled with nitrogen was charged with a solution of 4-(5-bromo-3-methylbenzo[b]thiophen-2-yl)pyrimidin-2-amine (1.2 g, 3.72 mmol) and THF (10 mL). To this was added n-butyl lithium (4.5 mL, 2.5M in hexanes) dropwise at −78° C. The reaction mixture was then saturated with CO₂(solid) and stirred at −78° C. for 3 hours. The reaction was the quenched by addition of concentrated hydrochloric acid (0.94 mL, 12M), concentrated, and extracted three times with EtOAc (20 mL). The crude product was recrystallized in methanol, resulting in 0.2 g (20% yield) of the title compound as a yellow solid. ¹H NMR (400 MHz, DMSO-d₆) δ:: 8.37 (s, 1H), 8.32 (d, 1H), 7.98 (d, 1H), 7.79 (d, 1H), 6.95 (d, 1H), 6.72 (s, 2H), 2.69 (s, 3H): LCMS (M+1)⁺: 286

### EXAMPLE 20

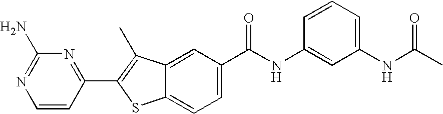

### N-(3-Acetamidophenyl)-2-(2-aminopyrimidin-4-yl)-3-methylbenzo[b]thiophene-5-carboxamide

A 50 mL round bottom flask was charged with 2-(2-aminopyrimidin-4-yl)-3-methylbenzo[b]thiophene-5-carboxylic acid (0.020 g, 0.07 mmol), N-(3-aminophenyl)acetamide (0.016 g, 0.1 mmol), TEA (0.020 g, 0.20 mmol), HATU (0.038 g, 0.1 mmol), and DMF. The resulting mixture was allowed to stir at room temperature for 2 hours. Work-up: the mixture was diluted with EtOAc (50 mL), washed three times with water (50 mL), brine (50 mL), and dried over Na₂SO₄. The crude material was purified by C18 reverse phase semi-preparative HPLC eluted with 10-100% acetonitrile in water in the presence of 0.1% TFA affording the title compound in 20 mg (69% yield) as an off-white solid. LCMS: (M+1)⁺: 417.91.

### EXAMPLE 21

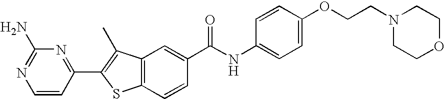

### 2-(2-Aminopyrimidin-4-yl)-3-methyl-N-(4-(2-morpholinoethoxy)phenyl)benzo[b]thiophene-5-carboxamide

The title compound was prepared analogously to N-(3-acetamidophenyl)-2-(2-aminopyrimidin-4-yl)-3-methylbenzo[b]thiophene-5-carboxamide, where 4-(2-morpholinoethoxy)aniline was substituted for N-(3-aminophenyl)acetamide as described in Example 20. LCMS (M+1)⁺: 490.04.

### EXAMPLE 22

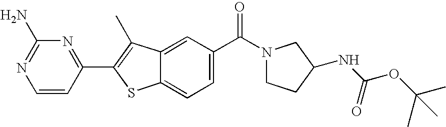

### tert-Butyl 1-(2-(2-aminopyrimidin-4-yl)-3-methylbenzo[b]thiophene-5-carbonyl)pyrrolidin-3-ylcarbamate

The title compound was prepared analogously to N-(3-acetamidophenyl)-2-(2-aminopyrimidin-4-yl)-3-methylbenzo[b]thiophene-5-carboxamide as described in Example 20, where tert-butyl pyrrolidin-3-ylcarbamate was substituted for N-(3-aminophenyl)acetamide. LCMS: (M+1)⁺: 453.98.

### EXAMPLE 23

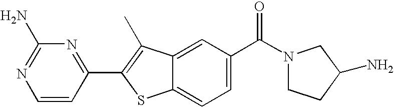

### (2-(2-Aminopyrimidin-4-yl)-3-methylbenzo[b]thiophen-5-yl)(3-aminopyrrolidin-1-yl)methanone

A 5 mL round bottom flask was charged with tert-butyl 1-(2-(2-aminopyrimidin-4-yl)-3-methylbenzo[b]thiophene-5-carbonyl)pyrrolidin-3-ylcarbamate (0.006 g, 0.013 mmol) in methylene chloride (1 mL), and trifluoroacetic acid (1 mL). The resulting mixture was stirred overnight at room temperature. The mixture was concentrated, and dissolved in methanol (1 mL). The crude product was purified by reverse phase C18 column chromatography eluted with 10-100% acetonitrile in water in the presence of 0.1% TFA affording the title compound in 3 mg (38% yield) as an off-white solid. LCMS: (M+1)⁺: 353.95.

### EXAMPLE 24

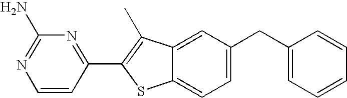

### 4-(5-Benzyl-3-methylbenzo[b]thiophen-2-yl)pyrimidin-2-amine

To a solution of 4-(5-bromo-3-methylbenzo[b]thiophen-2-yl)pyrimidin-2-amine (0.01 g, 0.03 mmol) in THF (0.3 mL) in a microwave reaction vessel, were added Pd(PPh₃)₂Cl₂ (0.002 g, 0.003 mmol), and CuI (0.001 g, 0.006 mmol). This mixture was degassed and back filled with nitrogen three times. To this mixture was added benzylzinc (II) bromide (0.0015 g, 0.12 mL THF solution, 0.5M) in one portion at room temperature. The microwaved at 150° C. for 5 minutes. Work up: the reaction was diluted with water (2 mL), extracted three times with ethyl acetate (100 mL), washed with brine, and dried over Na₂SO₄. The material was concentrated in vacuo to give the crude product that was purified by reverse phase C18 column chromatography eluted with 30-100% acetonitrile in water in the presence of 0.1% TFA. This afforded the title compound in 3 mg (29% yield) as a off-white solid. ¹H NMR (400 MHz, CD₃OD) δ: 7.80 (d, 1H), 7.77 (d, 1H), 7.35 (dd, 1H), 7.28-7.16 (m, 7H), 4.14 (s, 2H), 2.79 (s, 3H); LCMS: (M+1)⁺: 332.30.

### EXAMPLE 25

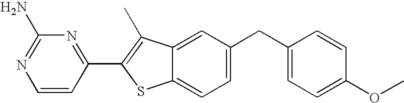

### 4-(5-(4-Methoxybenzyl)-3-methylbenzo[b]thiophen-2-yl)pyrimidin-2-amine

The title compound was prepared analogously to 4-(5-benzyl-3-methylbenzo[b]thiophen-2-yl)pyrimidin-2-amine as described in Example 24, where (4-methoxybenzyl)zinc (II) bromide was substituted for benzylzinc (II) bromide. ¹H NMR (400 MHz, CDCl₃) δ: 8.33 (d, 1H), 7.74 (d, 1H), 7.58 (d, 1H), 7.23 (dd, 1H), 7.14 (d, 2H), 6.98 (d, 1H), 6.84 (d, 2H), 5.21 (s, 2H), 4.07 (s, 2H), 3.78 (s, 3H), 2.68 (s, 3H). LCMS: (M+1)⁺: 362.61.

### EXAMPLE 26

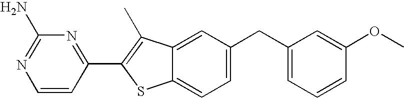

### 4-(5-(3-Methoxybenzyl)-3-methylbenzo[b]thiophen-2-yl)pyrimidin-2-amine

The title compound was prepared analogously to 4-(5-benzyl-3-methylbenzo[b]thiophen-2-yl)pyrimidin-2-amine as described in Example 24, where (3-methoxybenzyl)zinc (II) bromide was substituted for benzylzinc (II) bromide. ¹H NMR (400 MHz, CDCl₃) δ: 8.34 (d, 1H), 7.74 (d, 1H), 7.60 (d, 1H), 7.23-7.20 (m, 3H), 6.99 (d, 1H), 6.81 (d, 1H), 6.76-6.75 (m, 1H) 5.07 (s, 2H), 4.09 (s, 2H), 3.77 (s, 3H), 2.68 (3H). LCMS: (M+1)⁺: 362.20.

### EXAMPLE 27

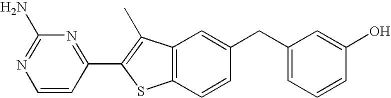

### 3-((2-(2-Aminopyrimidin-4-yl)-3-methylbenzo[b]thiophen-5-yl)methyl)phenol

The title compound was prepared analogously to 3-(2-(2-aminopyrimidin-4-yl)-3-methylbenzo[b]thiophen-5-yloxy)phenol, where 4-(5-(3-methoxybenzyl)-3-methylbenzo[b]thiophen-2-yl)pyrimidin-2-amine was substituted for 4-(5-(3-methoxyphenoxy)-3-methylbenzo[b]thiophen-2-yl)pyrimidin-2-amine as described in Example 82. ¹H NMR (400 MHz, DMSO-d₆) δ: 9.23 (s, 1H), 8.32 (d, 1H), 7.84 (d, 1H), 7.75 (s, 1H), 7.22 (d, 1H), 7.05 (t, 1H), 6.95 (d, 1H), 6.74 (s, 2H), 6.68 (d, 1H), 6.60 (s, 1H), 6.54 (d, 1H), 3.98 (s, 2H), 2.64 (s, 3H). LCMS (M+1)⁺: 348.13.

### EXAMPLE 28

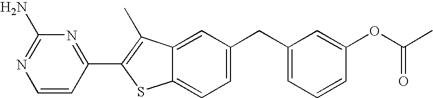

### 3-((2-(2-Aminopyrimidin-4-yl)-3-methylbenzo[b]thiophen-5-yl)methyl)phenyl acetate

A 20 mL screw cap vial was charged with 3-((2-(2-aminopyrimidin-4-yl)-3-methylbenzo[b]thiophen-5-yl)methyl)phenol (0.02 g, 0.057 mmol, prepared in Example 27), K₂CO₃ (0.008 g, 0.057 mmol), DMF (1.1 mL), and acetic anhydride (0.006 g, 0.057 mmol). The reaction mixture was then stirred at room temperature for 16 h and progress was monitored by LCMS. Work-up: the reaction mixture was extracted with EtOAc (3×50 mL) and the combined organic phases were washed with water and brine, then dried over Na₂SO₄ and evaporated. The crude material was purified by silica gel column chromatography eluting with EtOAc in hexanes to provide the title compound (22 mg, 98% yield) as an off-white solid. ¹H NMR (400 MHz, CD₃OD) δ: 8.26 (d, 1H), 7.82-7.78 (m, 2H), 7.36 (dd, 1H), 7.28 (d, 1H), 7.10 (t, 1H), 6.72 (d, 1H), 6.65-6.60 (m, 2H), 4.06 (s, 2H), 2.80 (s, 3H); LCMS: (M+1)⁺: 348.04.

### EXAMPLE 29

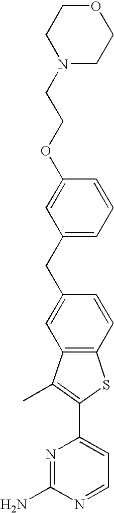

### 4-(3-Methyl-5-(3-(2-morpholinoethoxy)benzyl)benzo[b]thiophen-2-yl)pyrimidin-2-amine

An 8 mL screw cap vial was charged with 3-((2-(2-aminopyrimidin-4-yl)-3-methylbenzo[b]thiophen-5-yl)methyl)phenol (35 mg, 0.10 mmol, prepared as described in Example 27), 2-morpholinoethanol (0.024 mL, 0.20 mmol), triphenylphosphine (52 mg, 0.20 mmol), THF (1 mL) and di-tert-butyl azodicarboxylate (46 mg, 0.20 mmol), then stirred 16 h and evaporated. To the residue was added CH₂Cl₂ (1 mL) and TFA (0.5 mL) and the mixture was stirred for 2 h, then evaporated to dryness. The crude product was purified by C18 reverse phase semi-preparative HPLC, giving the product as a faintly yellow solid (bis TFA salt, 33 mg, 48%.) ¹H NMR (400 MHz, CD₃OD) δ: 8.27 (bs, 1H), 7.82 (m, 2H), 7.35 (m, 2H), 7.26 (m, 1H), 6.94 (m, 1H), 6.87 (m, 2H), 4.34 (m, 2H), 4.12 (s, 2H), 4.01 (bs, 2H), 3.80 (bs, 2H), 3.59 (m, 2H), 3.54 (bs, 2H), 3.25 (bs, 2H), 2.82 (s, 3H). LCMS (M+1⁺): 461.22.

### EXAMPLE 30

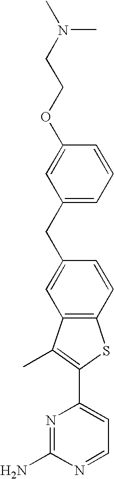

### 4-(5-(3-(2-(Dimethylamino)ethoxy)benzyl)-3-methylbenzo[b]thiophen-2-yl)pyrimidin-2-amine

The title compound was prepared analogously to 4-(3-methyl-5-(3-(2-morpholinoethoxy)benzyl)benzo[b]thiophen-2-yl)pyrimidin-2-amine in Example 29, where 2-(dimethylamino)ethanol was substituted for 2-morpholinoethanol. ¹H NMR (400 MHz, CD₃OD) δ: 8.26 (m, 1H), 7.82 (m, 2H), 7.37 (m, 1H), 7.33 (m, 1H), 7.26 (m, 1H), 6.94 (m, 1H), 6.88 (m, 2H), 4.30 (m, 2H), 4.13 (s, 2H), 3.55 (m, 2H), 2.94 (s, 6H), 2.83 (s, 3H). LCMS (M+1⁺): 419.17.

### EXAMPLE 31

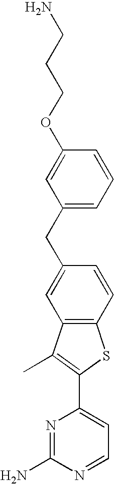

### 4-(5-(3-(3-Aminopropoxy)benzyl)-3-methylbenzo[b]thiophen-2-yl)pyrimidin-2-amine

The title compound was prepared analogously to 4-(3-methyl-5-(3-(2-morpholinoethoxy)benzyl)benzo[b]thiophen-2-yl)pyrimidin-2-amine in Example 29, where tert-butyl 3-hydroxypropylcarbamate was substituted for 2-morpholinoethanol. ¹H NMR (400 MHz, CD₃OD) δ: 8.24 (m, 1H), 7.81 (m, 2H), 7.36 (m, 1H), 7.32 (m, 1H), 7.22 (m, 1H), 6.83 (m, 3H), 4.10 (s, 2H), 4.07 (m, 2H), 3.12 (m, 2H), 2.82 (s, 3H), 2.11 (m, 2H). LCMS (M+11): 405.19.

### EXAMPLE 32

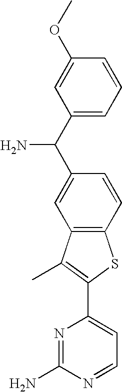

### 4-(5-(Amino(3-methoxyphenyl)methyl)-3-methylbenzo[b]thiophen-2-yl)pyrimidin-2-amine

Step 1

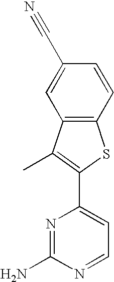

### 2-(2-Aminopyrimidin-4-yl)-3-methylbenzo[b]thiophene-5-carbonitrile

A 50 mL round bottom flask was charged with 4-(5-bromo-3-methylbenzo[b]thiophen-2-yl)pyrimidin-2-amine (1.28 g, 4.00 mmol, prepared as described in Example 13), zinc cyanide (258 mg, 2.20 mmol), bis(tri-tert-butylphosphine)palladium (90 mg, 0.18 mmol), and zinc (52 mg, 0.80 mmol), then evacuated and back-filled with nitrogen. N,N-Dimethylacetamide (20 mL) was added and the reaction vessel vacuum flushed with nitrogen three times. The mixture was placed in a 95° C. oil bath and stirred for 16 h. After cooling, the reaction mixture was filtered through Celite. To the filtrate was added 3 N NH₄OH (1.6 mL), and H₂O (80 mL). The resulting mixture was stirred for 2.5 h. Solid material formed and was collected by filtration, washed with water (60 mL) and air dried. The resulting solid was dissolved in hot THF (50 mL), and filtered. The filtrate was concentrated and purified by silica gel chromatography, eluting with EtOAc and hexanes to afford the title compound (650 mg, 61%) as a pale yellow solid. ¹H NMR (400 MHz, DMSO-d₆) δ: 8.48 (m, 1H), 8.37 (m, 1H), 8.20 (m, 1H), 7.28 (m, 1H), 7.02 (m, 1H), 6.84 (bs, 2H), 2.69 (s, 3H). LCMS (M+1⁺): 267.08.
Step 2

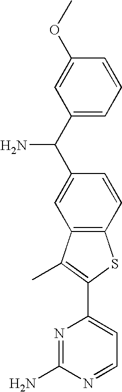

### 4-(5-(Amino(3-methoxyphenyl)methyl)-3-methylbenzo[b]thiophen-2-yl)pyrimidin-2-amine

A 20 mL screw cap vial was charged with 2-(2-aminopyrimidin-4-yl)-3-methylbenzo[b]thiophene-5-carbonitrile (67 mg, 0.25 mmol) and THF (1.25 mL). To this was added a solution of 3-methoxyphenylmagnesium bromide (1.0 M, 1.25 mL, 1.25 mmol). The reaction vessel was placed in 70° C. oil bath and stirred for 16 h, then allowed to cool. Methanol (2 mL) was added carefully, followed by NaBH₄ (28 mg, 0.74 mmol) and the reaction mixture was stirred for 1 h, then evaporated and partitioned between H₂O (20 mL) and EtOAc (3×30 mL). The combined organic phases were dried over Na₂SO₄ and evaporated. The crude product was purified by silica gel chromatography, eluting with 10% methanol in CH₂Cl₂ to afford the title compound (45 mg) as a film contaminated with an unknown impurity. LCMS (M+1⁺): 377.13.
Step 3

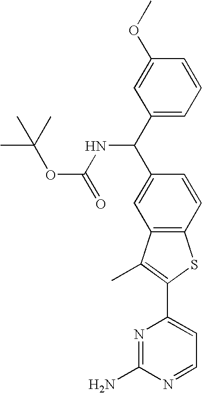

### tert-Butyl (2-(2-aminopyrimidin-4-yl)-3-methylbenzo[b]thiophen-5-yl)(3-methoxyphenyl)methylcarbamate

An 8 mL screw cap vial was charged with impure 4-(5-(amino(3-methoxyphenyl)methyl)-3-methylbenzo[b]thiophen-2-yl)pyrimidin-2-amine (43 mg, 0.11 mmol), triethylamine (0.032 mL, 0.23 mmol), methanol (0.5 mL), and THF (0.5 mL). Di-tert-butyl dicarbonate (25 mg, 0.11 mmol) was added and the reaction mixture was stirred for 1 h, then evaporated and the crude product was purified by silica gel chromatography, eluting with 10% methanol and CH₂Cl₂ to afford the title compound (32 mg, 59%) as a film. ¹H NMR (400 MHz, DMSO-d₆) δ: 8.32 (m, 1H), 8.04 (bd, 1H), 7.89 (m, 2H), 7.41 (m, 1H), 7.20 (m, 1H), 6.94 (m, 3H), 6.76 (m, 3H), 5.93 (bd, 1H), 3.70 (s, 3H), 2.65 (s, 3H), 1.39 (bs, 9H). LCMS (M+1⁺): 477.25.
Step 4

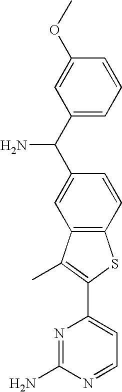

### 4-(5-(Amino(3-methoxyphenyl)methyl)-3-methylbenzo[b]thiophen-2-yl)pyrimidin-2-amine

A 25 mL round bottom flask was charged with tert-butyl (2-(2-aminopyrimidin-4-yl)-3-methylbenzo[b]thiophen-5-yl)(3-methoxyphenyl)methylcarbamate (10 mg, 0.021 mmol), CH₂Cl₂ (2 mL) and TFA (1 mL). After stirring 75 min, the reaction mixture was evaporated to dryness giving the title compound as a yellow film (bis TFA salt, 7.7 mg, 48%). ¹H NMR (400 MHz, DMSO-d₆) δ: 8.95 (b, 3H), 8.36 (m, 1H), 8.10 (m, 1H), 8.03 (m, 1H), 7.48 (m, 1H), 7.36 (m, 1H), 7.14 (m, 1H), 7.04 (m, 2H), 6.93 (m, 2H), 5.78 (bm, 1H), 3.75 (s, 3H), 2.69 (s, 3H). LCMS (M+1⁺): 377.14.

### EXAMPLE 33

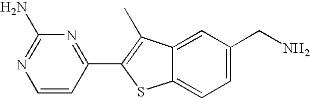

### 4-(5-(Aminomethyl)-3-methylbenzo[b]thiophen-2-yl)pyrimidin-2-amine

A 50 mL round bottom flask was charged with a solution of 2-(2-aminopyrimidin-4-yl)-3-methylbenzo[b]thiophene-5-carbonitrile (500 mg, 1.88 mmol) prepared as described in Example 32, in THF (20 mL). To this mixture was added LiAlH₄ (300 mg, 7.89 mmol). The resulting mixture was heated to 60° C. overnight. After cooling to room temperature, the reaction mixture was quenched by addition of 10 mL of water/ice. The resulting solution was extracted three times with EtOAc (50 mL), washed with brine, dried over Na₂SO₄, and concentrated to afford the product in 0.5 g (91% yield) as a white solid. ¹H NMR (300 MHz, CD₃OD) δ:: 8.31 (d, 1H), 7.85 (s, 1H), 7.83 (d, 1H), 7.43 (d, 1H), 7.05 (d, 1H), 3.95 (s, 2H), 2.76 (s, 3H).

### EXAMPLE 34

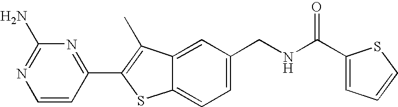

### N-((2-(2-Aminopyrimidin-4-yl)-3-methylbenzo[b]thiophen-5-yl)methyl)thiophene-2-carboxamide

A 50 mL round bottom flask was charged with 4-(5-(aminomethyl)-3-methylbenzo[b]thiophen-2-yl)pyrimidin-2-amine (0.025 g, 0.09 mmol) prepared as described in Example 33, thiophene-2-carboxylic acid (0.013 g, 0.1 mmol), TEA (0.018 g, 0.18 mmol), and HATU (0.051 g) in DMF. The resulting mixture was allowed to stir at room temperature for 4 h. Work-up: the mixture was washed with water (50 mL), extracted three times with EtOAc (25 mL), washed with brine (50 mL), and dried over Na₂SO₄. The mixture was concentrated, and purified by reverse phase C18 column chromatography eluted with 10-100% acetonitrile in water in the presence of 0.1% TFA affording the product in 10 mg (27% yield) as an off white solid. LCMS: (M+1)⁺: 380.90.

### EXAMPLE 35

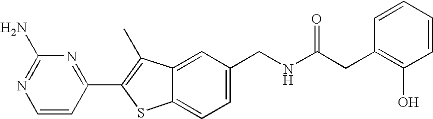

### N-((2-(2-Aminopyrimidin-4-yl)-3-methylbenzo[b]thiophen-5-yl)methyl)-2-(2-hydroxyphenyl)acetamide

The title compound was prepared analogously to N-((2-(2-aminopyrimidin-4-yl)-3-methylbenzo[b]thiophen-5-yl)methyl)thiophene-2-carboxamide as described in Example 34, where 2-(2-hydroxyphenyl)acetic acid was substituted for thiophene-2-carboxylic acid in that procedure. LCMS: (M+1)⁺: 404.92.

### EXAMPLE 36

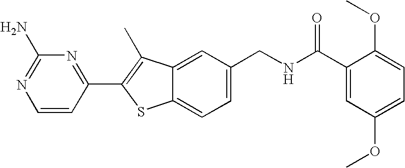

### N-((2-(2-Aminopyrimidin-4-yl)-3-methylbenzo[b]thiophen-5-yl)methyl)-2,5-dimethoxybenzamide

The title compound was prepared analogously to N-((2-(2-aminopyrimidin-4-yl)-3-methylbenzo[b]thiophen-5-yl)methyl)thiophene-2-carboxamide as described in Example 34, where 2,5-dimethoxybenzoic acid was substituted for thiophene-2-carboxylic acid in that procedure. LCMS: (M+1)⁺: 434.94.

### EXAMPLE 37

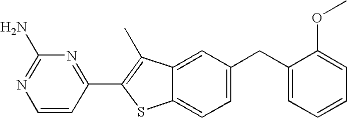

### 4-(5-(2-Methoxybenzyl)-3-methylbenzo[b]thiophen-2-yl)pyrimidin-2-amine

The title compound was prepared analogously to 4-(5-benzyl-3-methylbenzo[b]thiophen-2-yl)pyrimidin-2-amine as described in Example 24, where (2-methoxybenzyl)zinc (II) bromide was substituted for benzylzinc (II) bromide. ¹H NMR (400 MHz, CDCl₃) δ: 7.73 (d, 1H), 7.63 (d, 1H), 7.28 (dd, 1H), 7.26-7.20 (m, 2H), 7.08 (dd, 1H), 6.98 (d, 1H), 6.90-6.86 (m, 2H) 5.13 (s, 2H), 4.11 (s, 2H), 3.84 (s, 3H), 2.67 (s, 3H). LCMS: (M+1)⁺: 361.80.

### EXAMPLE 38

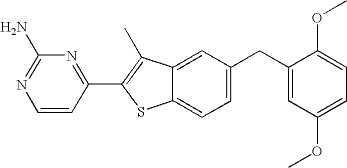

### 4-(5-(2,5-Dimethoxybenzyl)-3-methylbenzo[b]thiophen-2-yl)pyrimidin-2-amine

The title compound was prepared analogously to 4-(5-benzyl-3-methylbenzo[b]thiophen-2-yl)pyrimidin-2-amine as described in Example 24, where (2,5-dimethoxybenzyl)zinc (II) bromide was substituted for benzylzinc (II) bromide. ¹H NMR (400 MHz, CDCl₃) δ: 8.34 (d, 1H), 7.73 (d, 1H), 7.62 (d, 1H), 7.28 (dd, 1H), 6.82 (d, 1H), 6.73-6.66 (m, 2H) 5.10 (s, 2H), 4.08 (s, 2H), 3.79 (s, 3H), 3.71 (s, 3H), 2.67 (s, 3H). LCMS: (M+1)⁺: 391.83.

### EXAMPLE 39

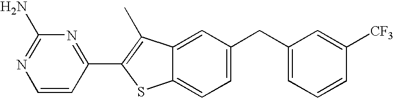

### 4-(3-Methyl-5-(3-(trifluoromethyl)benzyl)benzo[b]thiophen-2-yl)pyrimidin-2-amine

The title compound was prepared analogously to 4-(5-benzyl-3-methylbenzo[b]thiophen-2-yl)pyrimidin-2-amine as described in Example 24, where (3-(trifluoromethyl)benzyl) zinc (II) bromide was substituted for benzylzinc (II) bromide. ¹H NMR (400 MHz, CDCl₃) δ: 8.36 (d, 1H), 7.78 (d, 1H), 7.59 (d, 1H), 7.50-7.47 (m, 4H), 7.21 (d, 1H), 6.99-6.97 (m, 1H) 5.12 (s, 2H), 4.18 (s, 2H), 2.68 (s, 3H). LCMS: (M+1)⁺: 400.03.

### EXAMPLE 40

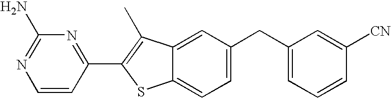

### 3-((2-(2-Aminopyrimidin-4-yl)-3-methylbenzo[b]thiophen-5-yl)methyl)benzonitrile

The title compound was prepared analogously to 4-(5-benzyl-3-methylbenzo[b]thiophen-2-yl)pyrimidin-2-amine as described in Example 24, where (3-cyanobenzyl) zinc (II) bromide was substituted for benzylzinc (II) bromide. ¹H NMR (400 MHz, CDCl₃) δ: 8.36 (d, 1H), 7.78 (d, 1H), 7.57 (d, 1H), 7.52-7.26 (m, 4H), 7.19 (d, 1H), 6.99 (d, 1H) 5.08 (s, 2H), 4.15 (s, 2H), 2.69 (s, 3H). LCMS: (M+1)⁺: 357.04.

### EXAMPLE 41

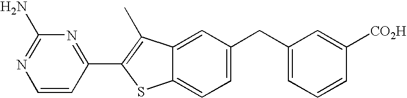

### 3-((2-(2-Aminopyrimidin-4-yl)-3-methylbenzo[b]thiophen-5-yl)methyl)benzoic acid

A 25 mL round bottom flask was charged with 3-((2-(2-aminopyrimidin-4-yl)-3-methylbenzo[b]thiophen-5-yl)methyl)benzonitrile (0.040 g, 0.11 mmol, prepared in Example 40), methanol (2.2 mL), and NaOH aq. (2 M, 2.3 mL), then refluxed overnight. Work-up: the reaction was concentrated, suspended in EtOH, pH adjusted to 5 by addition of concentrated HCl aq. A white precipitate formed that was collected by filtration, washed with EtOH, then purified by C18 reverse phase semi-preparative HPLC, giving the title compound (0.02 g, 48% yield) as an off-white solid. ¹H NMR (400 MHz, CD₃OD) δ: 8.26 (d, 1H), 7.78-7.73 (m, 3H), 7.72-7.70 (m, 1H), 7.46-7.36 (m, 3H), 7.26 (m, 1H), 4.20 (s, 2H), 2.80 (s, 3H); LCMS: (M+1)⁺: 375.02.

### EXAMPLE 42

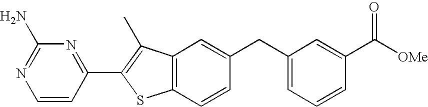

### Methyl 3-((2-(2-aminopyrimidin-4-yl)-3-methylbenzo[b]thiophen-5-yl)methyl)benzoate

A flask was charged with 3-((2-(2-aminopyrimidin-4-yl)-3-methylbenzo[b]thiophen-5-yl)methyl)benzoic acid (16.0 mg, 0.0426 mmol, prepared as described in Example 41), (trimethylsilyl)diazomethane (2.0 M solution in Et₂O, 4.9 mg, 0.0426 mmol), and THF:methanol (0.5 mL, 1:1). The resulting mixture was stirred overnight at room temperature. The mixture was concentrated, and then purified by SiO₂ flash chromatography, eluting with 10% methanol and methylene chloride to afford the title compound in 7.2 mg (43% yield), as an off white solid. ¹H NMR (400 MHz, DMSO-d₆) δ: 8.38 (d, 1H), 7.89-7.78 (m, 4H), 7.59 (d, 1H), 7.44 (t, 1H), 7.39 (d, 1H), 6.97 (d, 1H), 6.73 (s, 2H), 4.18 (s, 2H), 3.81 (s, 3H), 2.64 (s, 3H). LCMS (M+1)⁺: 390.11.

### EXAMPLE 43

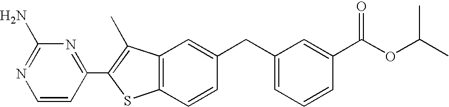

### Isopropyl 3-((2-(2-aminopyrimidin-4-yl)-3-methylbenzo[b]thiophen-5-yl)methyl)benzoate

A 5 mL round bottom flask was charged with 3-((2-(2-aminopyrimidin-4-yl)-3-methylbenzo[b]thiophen-5-yl)methyl)benzoic acid (24.4 mg, 0.0650 mmol) prepared as described in Example 41 in 1.0 M solution of H₂SO₄ in i-propanol (1 mL). The resulting mixture was stirred overnight at 92° C. Work-up: the mixture was diluted with EtOAc (50 mL), washed with saturated aqueous NaHCO₃ (50 mL), washed three times with water (50 mL), brine (50 mL), and dried over Na₂SO₄. The mixture was concentrated, and then purified by SiO₂ flash chromatography, eluting with 10% methanol and methylene chloride to afford the title compound in 5.9 mg (22% yield), as an off white solid ¹H NMR (400 MHz, CD₃OD) δ: 8.38 (d, 1H), 7.94-7.75 (m, 4H), 7.56 (d, 1H), 7.41 (t, 1H), 7.36 (d, 1H), 7.01 (d, 1H), 5.20 (m, 1H), 4.18 (s, 2H), 2.67 (s, 3H), 1.35 (d, 6H). LCMS (M+1)⁺: 418.18.

### EXAMPLE 44

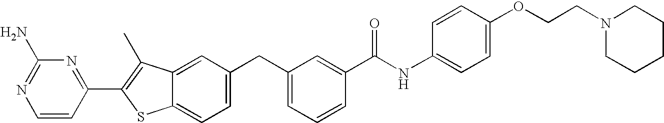

### 3-((2-(2-Aminopyrimidin-4-yl)-3-methylbenzo[b]thiophen-5-yl)methyl)-N-(4-(2-(piperidin-1-yl)ethoxy)phenyl)benzamide

A 20 mL screw cap vial was charged with 3-((2-(2-aminopyrimidin-4-yl)-3-methylbenzo[b]thiophen-5-yl)methyl)benzoic acid (0.050 g, 0.13 mmol, prepared in Example 41), 4-(2-(piperidin-1-yl)ethoxy)aniline (0.029 g, 0.1 mmol), triethylamine (0.026 g, 0.26 mmol), HATU (0.049 g, 0.13 mmol) and DMF. After stirring 2 h, LCMS analysis showed the reaction was complete. Work-up: water was added and the mixture was extracted with EtOAc (3×25 mL). The combined organic phases were washed with water and brine, then dried over Na₂SO₄ and evaporated. The crude product was purified by C18 reverse phase semi-preparative HPLC, giving the title compound (30 mg, 35% yield) as an off-white solid. ¹H NMR (400 MHz, CD₃OD) δ: 8.20 (d, 1H), 7.83 (s, 2H), 7.76 (dd, 1H), 7.61-7.59 (m, 2H), 7.49-7.38 (m, 3H), 7.29 (d, 1H), 7.02-6.99 (m, 2H), 4.38 (t, 2H), 4.21 (s, 2H), 4.10-3.70 (m, 4H), 3.63 (t, 2H), 3.62-3.57 (m, 2H), 2.80 (s, 3H); LCMS: (M+1)⁺: 580.17.

### EXAMPLE 45

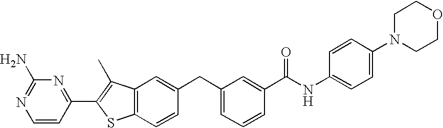

### 3-((2-(2-Aminopyrimidin-4-yl)-3-methylbenzo[b]thiophen-5-yl)methyl)-N-(4-morpholinophenyl)benzamide

A flask was charged with 3-((2-(2-aminopyrimidin-4-yl)-3-methylbenzo[b]thiophen-5-yl)methyl)benzoic acid (9.8 mg, 0.0261 mmol), prepared as described in Example 41, 4-morpholinoaniline (5.1 mg, 0.0287 mmol), HATU (10.9 mg, 0.0287 mmol), triethylamine (7.9 mg, 0.0783 mmol), in DMF (0.2 mL). The resulting mixture was stirred overnight at room temperature. Work-up: the mixture was diluted with EtOAc (50 mL), washed three times with water (50 mL), brine (50 mL), and dried over Na₂SO₄. The crude material was purified by C18 reverse phase semi-preparative HPLC, giving the product as white solid (mono TFA salt, 2.4 mg, 17% yield). ¹H NMR (400 MHz, DMSO-d₆) δ: 10.04 (s, 1H), 8.35 (d, 1H), 7.91-7.84 (m, 3H), 7.75 (d, 1H), 7.59 (m, 2H), 7.48-7.35 (m, 3H), 7.06 (d, 1H), 6.93 (d, 2H), 4.17 (s, 2H), 3.73 (t, 4H), 3.06 (t, 4H), 2.70 (s, 3H). LCMS (M+1)⁺: 536.12.

### EXAMPLE 46

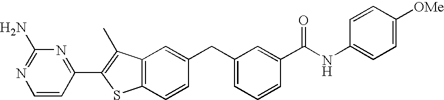

### 3-((2-(2-Aminopyrimidin-4-yl)-3-methylbenzo[b]thiophen-5-yl)methyl)-N-(4-methoxyphenyl)benzamide

The title compound was prepared analogously to 3-((2-(2-aminopyrimidin-4-yl)-3-methylbenzo[b]thiophen-5-yl)methyl)-N-(4-morpholinophenyl)benzamide, where p-anisidine was substituted for 4-morpholinoaniline as described in Example 45. ¹H NMR (400 MHz, CD₃OD) δ: 8.24 (d, 1H), 7.84 (m, 3H), 7.79 (d, 1H), 7.54-7.41 (m, 5H), 7.32 (d, 1H), 6.91 (d, 2H), 4.24 (s, 2H), 3.79 (s, 3H), 2.83 (s, 3H). LCMS (M+1)⁺: 481.01.

### EXAMPLE 47

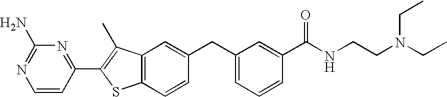

### 3-((2-(2-Aminopyrimidin-4-yl)-3-methylbenzo[b]thiophen-5-yl)methyl)-N-(2-(diethylamino)ethyl)benzamide

The title compound was prepared analogously to 3-((2-(2-aminopyrimidin-4-yl)-3-methylbenzo[b]thiophen-5-yl)methyl)-N-(4-morpholinophenyl)benzamide, where N,N-diethylethylenediamine was substituted for 4-morpholinoaniline as described in Example 45. ¹H NMR (400 MHz, CD₃OD) δ: 8.26 (d, 1H), 7.84-7.69 (m, 4H), 7.51-7.30 (m, 4H), 4.24 (s, 2H), 3.72 (t, 2H), 3.37-3.26 (m, 6H), 2.82 (s, 3H), 1.33 (t, 6H). LCMS (M+1)⁺: 474.63.

### EXAMPLE 48

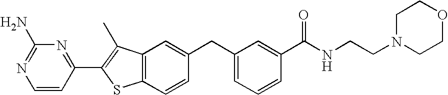

### 3-((2-(2-Aminopyrimidin-4-yl)-3-methylbenzo[b]thiophen-5-yl)methyl)-N-(3-morpholinoethyl)benzamide

The title compound was prepared analogously to 3-((2-(2-aminopyrimidin-4-yl)-3-methylbenzo[b]thiophen-5-yl)methyl)-N-(4-morpholinophenyl)benzamide, where 2-morpholinoethylamime was substituted for 4-morpholinoaniline as described in Example 45. ¹H NMR (400 MHz, CD₃OD) δ: 8.26 (d, 1H), 7.84-7.71 (m, 4H), 7.51-7.32 (m, 4H), 4.22 (s, 2H), 4.05 (m, 2H), 3.76 (t, 2H), 3.65 (m, 2H), 3.38 (t, 2H), 3.26 (m, 2H), 2.83 (s, 3H). LCMS (M+1)⁺: 488.62.

### EXAMPLE 49

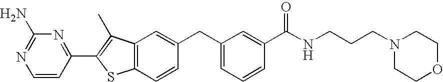

### 3-((2-(2-Aminopyrimidin-4-yl)-3-methylbenzo[b]thiophen-5-yl)methyl)-N-(3-morpholinopropyl)benzamide

The title compound was prepared analogously to 3-((2-(2-aminopyrimidin-4-yl)-3-methylbenzo[b]thiophen-5-yl)methyl)-N-(4-morpholinophenyl)benzamide, where 4-(3-aminopropyl)-morpholine was substituted for 4-morpholinoaniline as described in Example 45. ¹H NMR (400 MHz, CD₃OD) δ: 8.25 (d, 1H), 7.84-7.68 (m, 4H), 7.49-7.32 (m, 4H), 4.21 (s, 2H), 4.05-4.03 (m, 2H), 3.76 (t, 2H), 3.48 (t, 2H), 3.20 (t, 2H), 3.15-3.10 (m, 2H), 2.83 (s, 3H), 2.05 (m, 2H). LCMS (M+1)⁺: 502.64.

### EXAMPLE 50

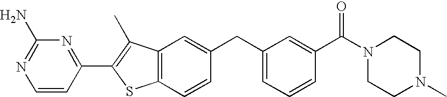

### (3-((2-(2-Aminopyrimidin-4-yl)-3-methylbenzo[b]thiophen-5-yl)methyl)phenyl)(4-methylpiperazin-1-yl)methanone

The title compound was prepared analogously to 3-((2-(2-aminopyrimidin-4-yl)-3-methylbenzo[b]thiophen-5-yl)methyl)-N-(4-morpholinophenyl)benzamide, where 1-methylpiperazine was substituted for 4-morpholinoaniline as described in Example 45. ¹H NMR (400 MHz, CD₃OD) δ: 8.35 (d, 1H), 7.91-7.85 (m, 2H), 7.46-7.26 (m, 5H), 7.06 (d, 1H), 4.14 (s, 2H), 3.29 (m, 4H), 3.04 (m, 4H), 2.79 (s, 3H), 2.69 (s, 3H). LCMS (M+1)⁺: 458.17.

### EXAMPLE 51

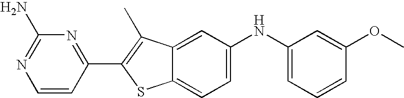

### 4-(5-(3-Methoxyphenylamino)-3-methylbenzo[b]thiophen-2-yl)pyrimidin-2-amine

A 20 mL screw cap vial was charged with 4-(5-bromo-3-methylbenzo[b]thiophen-2-yl)pyrimidin-2-amine (0.128 g, 0.4 mmol, prepared in Example 13), 3-methoxyaniline (0.1 g, 0.8 mmol), tert-BuONa (0.19 g, 2 mmol), 1,3-bis(2,6-di-1-propylphenyl)imidazolium chloride (0.034 g, 0.08 mmol) and Pd₂(dba)₃ (0.023 g, 0.04 mmol). This mixture was degassed and back filled with nitrogen three times, then heated to 95-100° C. overnight. Reaction progress was monitored by LCMS. Work-up: after cooling to room temperature, water (10 mL) was added and the mixture was extracted with EtOAc (3×100 mL). The combined organic phases were washed with water and brine, then dried over Na₂SO₄ and evaporated. The crude product was purified by silica gel chromatography, eluting with 10% methanol in CH₂Cl₂ to afford the title compound (70 mg, 48% yield) as a yellow solid. ¹H NMR (400 MHz, DMSO-d₆) δ: 8.31 (d, 2H), 8.09 (s, 1H), 7.79 (d, 1H), 7.50 (d, 1H), 7.20 (dd, 1H), 7.13 (t, 1H), 6.92 (d, 1H), 6.73 (s, 2H), 6.69-6.63 (m, 2H), 6.39 (dd, 1H), 3.70 (s, 3H), 2.57 (s, 3H); LCMS: (M+1)⁺: 363.02.

### EXAMPLE 52

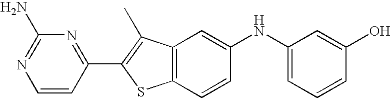

### 3-(2-(2-Aminopyrimidin-4-yl)-3-methylbenzo[b]thiophen-5-ylamino)phenol

The title compound was prepared analogously to 3-(2-(2-aminopyrimidin-4-yl)-3-methylbenzo[b]thiophen-5-yloxy)phenol, where 4-(5-(3-methoxyphenylamino)-3-methylbenzo[b]thiophen-2-yl)pyrimidin-2-amine was substituted for 4-(5-(3-methoxyphenoxy)-3-methylbenzo[b]thiophen-2-yl)pyrimidin-2-amine as described in Example 82. ¹H NMR (400 MHz, CD₃OD) δ: 8.27 (d, 1H), 7.69 (d, 1H), 7.53 (d, 1H), 7.23 (d, 1H), 7.20 (d, 1H), 7.04 (t, 1H), 6.99 (d, 1H), 6.62 (t, 1H), 6.59 (dd, 1H), 6.31 (dd, 1H), 2.65 (s, 3H). LCMS (M+1)⁺: 349.05.

### EXAMPLE 53

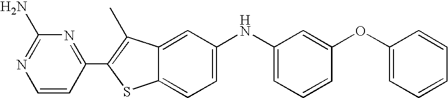

### 4-(3-Methyl-5-(3-phenoxyphenylamino)benzo[b]thiophen-2-yl)pyrimidin-2-amine

The title compound was prepared analogously to 4-(5-(3-methoxyphenylamino)-3-methylbenzo[b]thiophen-2-yl)pyrimidin-2-amine in Example 51, where 3-phenoxyaniline was substituted for 3-methoxyaniline. ¹H NMR (400 MHz, CD₃OD) δ: 8.24 (d, 1H), 7.74 (d, 1H), 7.76 (d, 1H), 7.37-7.33 (m, 2H), 7.28-7.19 (m, 3H), 7.10 (t, 1H), 7.07-7.02 (m, 2H), 6.86 (dd, 1H), 6.75 (t, 1H), 6.49 (dd, 1H), 2.72 (s, 3H). LCMS: (M+1)⁺: 425.00.

### EXAMPLE 54

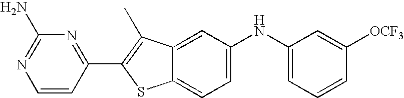

### 4-(3-Methyl-5-(3-(trifluoromethoxy)phenylamino)benzo[b]thiophen-2-yl)pyrimidin-2-amine

The title compound was prepared analogously to 4-(5-(3-methoxyphenylamino)-3-methylbenzo[b]thiophen-2-yl)pyrimidin-2-amine in Example 51, where 3-(trifluoromethoxy)aniline was substituted for 3-methoxyaniline. ¹H NMR (400 MHz, DMSO-d₆) δ: 8.63 (s, 1H), 8.34 (d, 1H), 7.88 (d, 1H), 7.57 (d, 1H), 7.32 (t, 1H), 7.24 (d, 1H), 7.09-6.96 (m, 4H), 6.72 (d, 1H), 2.61 (s, 3H). LCMS: (M+1)⁺: 416.86.

### EXAMPLE 55

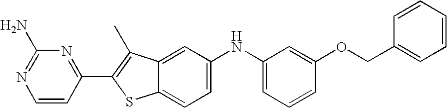

### 4-(5-(3-(Benzyloxy)phenylamino)-3-methylbenzo[b]thiophen-2-yl)pyrimidin-2-amine

The title compound was prepared analogously to 4-(5-(3-methoxyphenylamino)-3-methylbenzo[b]thiophen-2-yl)pyrimidin-2-amine in Example 51, where 3-(benzyloxy)aniline was substituted for 3-methoxyaniline. ¹H NMR (400 MHz, DMSO-d₆) δ: 8.34 (d, 1H), 7.81 (d, 1H), 7.51 (d, 1H), 7.43-7.02 (m, 9H), 6.70-6.69 (m, 2H), 6.48 (dd, 1H), 5.05 (s, 2H), 2.62 (s, 3H). LCMS: (M+1)⁺: 439.03.

### EXAMPLE 56

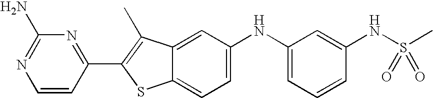

### N-(3-(2-(2-Aminopyrimidin-4-yl)-3-methylbenzo[b]thiophen-5-ylamino)phenyl)methanesulfonamide

The title compound was prepared analogously to 4-(5-(3-methoxyphenylamino)-3-methylbenzo[b]thiophen-2-yl)pyrimidin-2-amine in Example 51, where N-(3-aminophenyl)methanesulfonamide was substituted for 3-methoxyaniline. LCMS: (M+1)⁺: 425.95.

### EXAMPLE 57

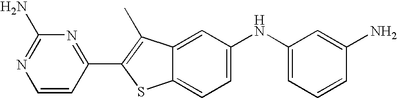

### N¹-(2-(2-Aminopyrimidin-4-yl)-3-methylbenzo[b]thiophen-5-yl)benzene-1,3-diamine

The title compound was prepared analogously to 4-(5-(3-methoxyphenylamino)-3-methylbenzo[b]thiophen-2-yl)pyrimidin-2-amine in Example 51, where benzene-1,3-diamine was substituted for 3-methoxyaniline. ¹H NMR (400 MHz, DMSO-d₆) δ: 8.51 (s, 1H), 8.34 (d, 1H), 7.87 (d, 1H), 7.54 (d, 1H), 7.25-7.22 (m, 2H), 7.02 (d, 1H), 6.94-6.92 (m, 2H), 6.60 (d, 1H), 2.63 (s, 3H). LCMS: (M+1)⁺: 348.04.

### EXAMPLE 58

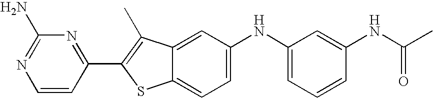

### N-(3-(2-(2-Aminopyrimidin-4-yl)-3-methylbenzo[b]thiophen-5-ylamino)phenyl)acetamide

The title compound was prepared analogously to 4-(5-(3-methoxyphenylamino)-3-methylbenzo[b]thiophen-2-yl)pyrimidin-2-amine in Example 51, where N-(3-aminophenyl)acetamide was substituted for 3-methoxyaniline. ¹H NMR (400 MHz, CD₃OD) δ: 8.22 (d, 1H), 7.74 (d, 1H), 7.66 (s, 2H), 7.31-7.29 (m, 2H), 7.18 (t, 1H), 6.86 (dd, 2H), 2.78 (s, 2H), 2.10 (s, 3H). LCMS: (M+1)⁺: 390.04.

### EXAMPLE 59

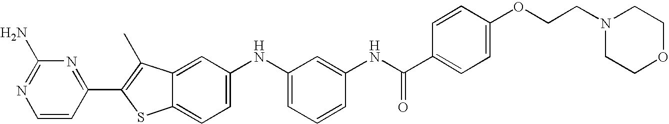

### N-(3-(2-(2-Aminopyrimidin-4-yl)-3-methylbenzo[b]thiophen-5-ylamino)phenyl)-4-(2-morpholinoethoxy)benzamide

A 4 mL screw cap vial was charged with N¹-(2-(2-aminopyrimidin-4-yl)-3-methylbenzo[b]thiophen-5-yl)benzene-1,3-diamine (0.050 g, 0.14 mmol, prepared in Example 57), 4-(2-morpholinoethoxy)benzoic acid (0.036 g, 0.14 mmol), triethylamine (0.042 g, 0.42 mmol), HATU (0.053 g, 0.13 mmol) and DMF. The reaction mixture was stirred overnight and progress was monitored by LCMS. Work-up: water was added and the mixture was extracted with EtOAc (3×25 mL). The combined organic phases were washed with water, brine, then dried over Na₂SO₄, and evaporated. The crude product was purified by C18 reverse phase semi-preparative HPLC, giving the title compound (29 mg, 35% yield) as a brown solid. ¹H NMR (400 MHz, CD₃OD) δ: 8.21 (d, 1H), 7.96-7.94 (m, 2H), 7.77-7.75 (m, 2H), 7.68 (d, 1H), 7.35 (dd, 1H), 7.32 (d, 1H), 7.24 (d, 1H), 7.13-7.05 (m, 2H), 6.92-6.89 (m, 1H), 4.48 (t, 2H), 4.06-4.05 (m, 2H), 3.82 (m, 2H), 3.67 (t, 2H), 3.59 (m, 2H), 3.32 (m, 2H), 2.80 (s, 3H); LCMS: (M+1)⁺: 581.18.

### EXAMPLE 60

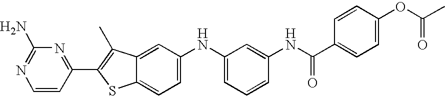

### 4-(3-(2-(2-Aminopyrimidin-4-yl)-3-methylbenzo[b]thiophen-5-ylamino)phenyl carbamoyl)phenyl acetate

The title compound was prepared analogously to N-(3-(2-(2-aminopyrimidin-4-yl)-3-methylbenzo[b]thiophen-5-ylamino)phenyl)-4-(2-morpholinoethoxy)benzamide in Example 59, where 4-acetoxybenzoic acid was substituted for 4-(2-morpholinoethoxy)benzoic acid. LCMS: (M+1)⁺: 510.05.

### EXAMPLE 61

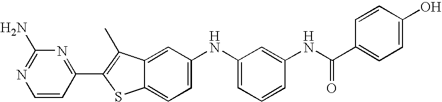

### N-(3-(2-(2-Aminopyrimidin-4-yl)-3-methylbenzo[b]thiophen-5-ylamino)phenyl)-4-hydroxybenzamide

A 4 mL screw cap vial was charged with 4-(3-(2-(2-aminopyrimidin-4-yl)-3-methylbenzo[b]thiophen-5-ylamino)phenyl carbamoyl)phenyl acetate (0.020 g, 0.039 mmol, prepared in Example 60), and methanol (0.8 mL). Aqueous NaOH (2 M, 0.03 mL) was added and the reaction mixture was stirred overnight. Work-up: the reaction concentrated and purified by C18 reverse phase semi-preparative HPLC, giving the title compound (9 mg, 49% yield) as a brown solid. ¹H NMR (400 MHz, CD₃OD) δ: 8.23 (s, 1H), 7.83-7.74 (m, 4H), 7.69 (d, 1H), 7.33 (dd, 1H), 7.28 (d, 1H), 7.22 (d, 1H), 7.06-7.04 (m, 1H), 6.90-6.84 (m, 3H), 2.80 (s, 3H); LCMS: (M+1)⁺: 468.01.

### EXAMPLE 62

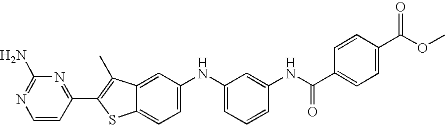

### Methyl 4-(3-(2-(2-aminopyrimidin-4-yl)-3-methylbenzo[b]thiophen-5-ylamino) phenylcarbamoyl)benzoate

The title compound was prepared analogously to N-(3-(2-(2-aminopyrimidin-4-yl)-3-methylbenzo[b]thiophen-5-ylamino)phenyl)-4-(2-morpholinoethoxy)benzamide in Example 60, where 4-(methoxycarbonyl)benzoic acid was substituted for 4-(2-morpholinoethoxy)benzoic acid. LCMS: (M+1)⁺: 510.10.

### EXAMPLE 63

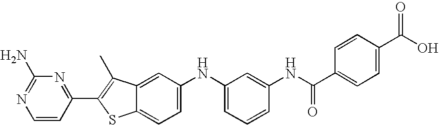

### 4-(3-(2-(2-Aminopyrimidin-4-yl)-3-methylbenzo[b]thiophen-5-ylamino) phenylcarbamoyl)benzoic acid

A 4 mL screw cap vial was charged with methyl 4-(3-(2-(2-aminopyrimidin-4-yl)-3-methylbenzo[b]thiophen-5-ylamino) phenylcarbamoyl)benzoate (0.010 g, 0.019 mmol, prepared in Example 62) and THF (0.4 mL). LiOH (0.5 mg) in water (0.1 mL) was added and the reaction mixture was stirred overnight. Work-up: after evaporation to dryness, the crude material was purified by C18 reverse phase semi-preparative HPLC, giving the title compound (4 mg, 41% yield) as an orange solid. LCMS: (M+1)⁺: 496.01.

### EXAMPLE 64

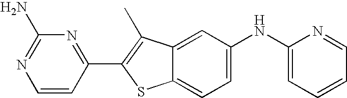

### 4-(3-Methyl-5-(pyridin-2-ylamino)benzo[b]thiophen-2-yl)pyrimidin-2-amine

The title compound was prepared analogously to 4-(5-(3-methoxyphenylamino)-3-methylbenzo[b]thiophen-2-yl)pyrimidin-2-amine in Example 51, where pyridin-2-amine was substituted for 3-methoxyaniline. ¹H NMR (400 MHz, CD₃OD) δ: 8.31 (d, 1H), 8.10-8.03 (m, 3H), 7.88 (d, 1H), 7.50 (dd, 1H), 7.32 (d, 1H), 7.24 (d, 1H), 7.05 (t, 1H), 2.84 (s, 3H). LCMS: (M+1)⁺: 334.02.

### EXAMPLE 65

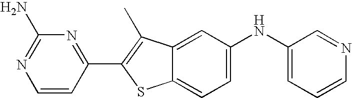

### 4-(3-Methyl-5-(pyridin-3-ylamino)benzo[b]thiophen-2-yl)pyrimidin-2-amine

The title compound was prepared analogously to 4-(5-(3-methoxyphenylamino)-3-methylbenzo[b]thiophen-2-yl)pyrimidin-2-amine in Example 51, where pyridin-3-amine was substituted for 3-methoxyaniline. ¹H NMR (400 MHz, CD₃OD) δ: 8.35 (d, 1H), 8.30 (d, 1H), 8.14 (d, 1H), 8.08 (ddd, 1H), 7.98 (d, 1H), 7.82 (s, 1H), 7.81 (dd, 1H), 7.44 (dd, 1H), 7.30 (d, 1H), 2.82 (s, 3H). LCMS: (M+1)⁺: 334.02.

### EXAMPLE 66

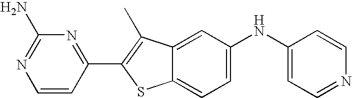

### 4-(3-Methyl-5-(pyridin-4-ylamino)benzo[b]thiophen-2-yl)pyrimidin-2-amine

The title compound was prepared analogously to 4-(5-(3-methoxyphenylamino)-3-methylbenzo[b]thiophen-2-yl)pyrimidin-2-amine in Example 51, where pyridin-4-amine was substituted for 3-methoxyaniline. ¹H NMR (400 MHz, CD₃OD) δ: 8.32 (d, 1H), 8.19-8.17 (m, 2H), 8.06 (d, 1H), 8.80 (d, 1H), 7.45 (d, 1H), 7.26 (d, 1H), 7.13 (m, 2H), 2.81 (s, 3H). LCMS: (M+1)⁺: 334.01.

### EXAMPLE 67

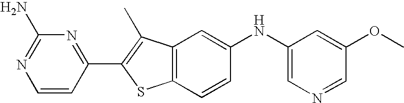

### 4-(5-(5-Methoxypyridin-3-ylamino)-3-methylbenzo[b]thiophen-2-yl)pyrimidin-2-amine

To a degassed solution of 4-(5-amino-3-methylbenzo[b]thiophen-2-yl)pyrimidin-2-amine (199 mg, 0.560 mmol, prepared as described in Example 72) in 1,4-dioxane (2 mL), was added 3-bromo-5-methoxypyridine (105 mg, 0.560 mmol), t-BuONa (269 mg, 2.80 mmol), 1,3-bis(2,6-di-i-propylphenyl)imidazolium chloride (47.6 mg, 0.112 mmol), and Pd₂(dba)₃ (32.2 mg, 0.0560 mmol), in that order. This mixture was then degassed and back filled with nitrogen three times. The resulting mixture was heated to 95° C. and stirred overnight. Upon completion as confirmed by LCMS, the reaction was cooled down to room temperature and quenched by addition of water (10 mL). This mixture was then extracted three times with ethyl acetate (100 mL), washed with water, brine and dried over Na₂SO₄. The resulting mixture was filtered, and the filtrate was concentrated, and purified by silica gel column chromatography eluted with 10% methanol and methylene chloride affording the title compound in 140.4 mg (69% yield) as a red solid. ¹H NMR (400 MHz, CD₃OD) δ: 8.28 (m, 1H), 7.91 (m, 1H), 7.78 (m, 1H), 7.68 (m, 1H), 7.57 (m, 1H), 7.26 (m, 1H), 7.08 (m, 1H), 7.00 (m, 1H), 3.83 (s, 3H), 2.66 (s, 3H). LCMS (M+1)⁺: 364.13

### EXAMPLE 68

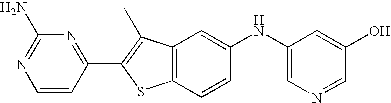

### 5-(2-(2-Aminopyrimidin-4-yl)-3-methylbenzo[b]thiophen-5-ylamino)pyridin-3-ol

The title compound was prepared analogously to 3-(2-(2-aminopyrimidin-4-yl)-3-methylbenzo[b]thiophen-5-yloxy)phenol, where 4-(5-(5-methoxypyridin-3-ylamino)-3-methylbenzo[b]thiophen-2-yl)pyrimidin-2-amine was substituted for 4-(5-(3-methoxyphenoxy)-3-methylbenzo[b]thiophen-2-yl)pyrimidin-2-amine as described in Example 82. ¹H NMR (400 MHz, CD₃OD) δ: 8.29 (m, 1H), 7.81 (m, 1H), 7.77 (s, 1H), 7.57 (m, 1H), 7.25 (m, 1H), 7.02 (m, 2H), 2.68 (s, 3H). LCMS (M+1)⁺: 350.14.

### EXAMPLE 69

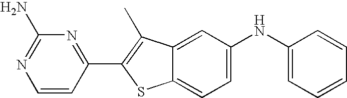

### 4-(3-Methyl-5-(phenylamino)benzo[b]thiophen-2-yl)pyrimidin-2-amine

The title compound was prepared analogously to 4-(5-(3-methoxyphenylamino)-3-methylbenzo[b]thiophen-2-yl)pyrimidin-2-amine in Example 51, where aniline was substituted for 3-methoxyaniline. ¹H NMR (400 MHz, DMSO-d₆) δ: 8.33 (d, 2H), 7.82 (d, 1H), 7.51 (d, 1H), 7.26-7.21 (m, 3H), 7.12-7.10 (m, 3H), 7.02 (m, 2H), 6.82 (t, 1H), 2.61 (s, 3H). LCMS: (M+1)⁺: 333.10.

### EXAMPLE 70

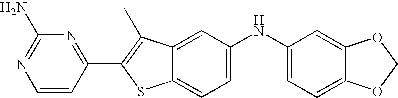

### 4-(5-(Benzo[d][1,3]dioxol-5-ylamino)-3-methylbenzo[b]thiophen-2-yl)pyrimidin-2-amine

The title compound was prepared analogously to 4-(5-(3-methoxyphenylamino)-3-methylbenzo[b]thiophen-2-yl)pyrimidin-2-amine in Example 51, where benzo[d][1,3]dioxol-5-amine was substituted for 3-methoxyaniline. ¹H NMR (400 MHz, CD₃OD) δ: 8.22 (d, 1H), 7.70 (d, 1H), 7.42 (d, 1H), 7.26 (d, 1H), 7.18 (dd, 1H), 6.67-6.62 (m, 2H), 6.64 (d, 1H), 5.91 (s, 2H), 2.74 (s, 3H). LCMS: (M+1)⁺: 377.03.

### EXAMPLE 71

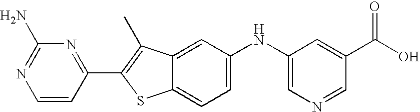

### 5-(2-(2-Aminopyrimidin-4-yl)-3-methylbenzo[b]thiophen-5-ylamino)nicotinic acid

The title compound was prepared analogously to 4-(5-(3-methoxyphenylamino)-3-methylbenzo[b]thiophen-2-yl)pyrimidin-2-amine in Example 51, where 5-aminonicotinic acid was substituted for 3-methoxyaniline. ¹H NMR (400 MHz, CD₃OD) δ: 8.60 (s, 2H), 8.51-8.46 (m, 2H), 8.29 (d, 1H), 8.01 (d, 1H), 7.88 (s, 1H), 7.49 (dd, 1H), 7.40 (d, 1H), 2.86 (s, 3H); LCMS: (M+1)⁺: 377.98.

### EXAMPLE 72

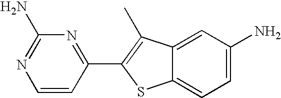

### 4-(5-Amino-3-methylbenzo[b]thiophen-2-yl)pyrimidin-2-amine

Step 1

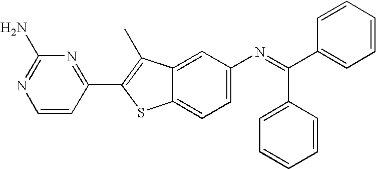

### 4-(5-(Diphenylmethyleneamino)-3-methylbenzo[b]thiophen-2-yl)pyrimidin-2-amine

A 20 mL screw cap vial was charged with 4-(5-bromo-3-methylbenzo[b]thiophen-2-yl)pyrimidin-2-amine (0.1 g, 0.3 mmol, prepared in Example 13), diphenylmethanimine (0.11 g, 0.62 mmol), Cs₂CO₃ (0.29 g, 0.9 mmol), BINAP (0.028 g, 0.045 mmol), Pd(OAc)₂ (0.010 g, 0.015 mmol) and toluene (1.5 ml). This mixture was then degassed and back filled with nitrogen three times, then heated to 100° C. overnight. Work-up: the reaction was diluted with water (10 mL) extracted with EtOAc (3×50 mL), brine, dried over Na₂SO₄, and evaporated giving the crude product which was used in the next step without further purification.
Step 2

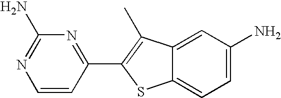

### 4-(5-Amino-3-methylbenzo[b]thiophen-2-yl)pyrimidin-2-amine

A 20 mL screw cap vial was charged with 4-(5-(diphenylmethyleneamino)-3-methylbenzo[b]thiophen-2-yl)pyrimidin-2-amine (0.1 g, 0.24 mmol), THF (2.4 mL) and aqueous HCl (1 M, 2.3 mL), then stirred for 6 h. Reaction progress was monitored by LCMS. Work-up: the reaction mixture was extracted with EtOAc (3×50 mL) and the combined organic phases were washed with water and brine, then dried over Na₂SO₄ and evaporated. The crude material was purified by C18 reverse phase semi-preparative HPLC, giving the title compound (51 mg, 83% yield) as a brown solid. LCMS: (M+1)⁺: 257.04.

### EXAMPLE 73

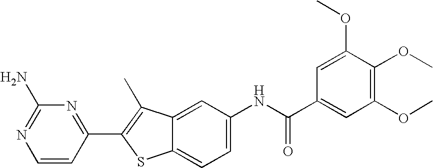

### N-(2-(2-Aminopyrimidin-4-yl)-3-methylbenzo[b]thiophen-5-yl)-3,4,5-trimethoxy benzamide

A 20 mL screw cap vial was charged with 4-(5-amino-3-methylbenzo[b]thiophen-2-yl)pyrimidin-2-amine (0.1 g, 0.39 mmol, prepared in Example 72), 3,4,5-trimethoxybenzoic acid (0.083 g, 0.39 mmol), triethylamine (0.11 g, 1.12 mmol) DMF and HATU (0.15 g, 0.39 mmol). The reaction mixture was stirred overnight and LCMS analysis showed complete conversion to product. Work-up: water was added, the mixture was extracted with EtOAc (3×25 mL) and the combined organic phases were washed with water and brine, then dried over Na₂SO₄ and evaporated. The crude material was purified by silica gel column chromatography eluting with EtOAc in hexanes to provide the title compound (0.13 g, 76% yield) as a yellow solid. ¹H NMR (400 MHz, DMSO-d₆) δ: 10.31 (s, 1H), 8.36-8.35 (m, 2H), 7.96 (d, 1H), 7.78 (dd, 1H), 7.32 (s, 2H), 7.05 (d, 1H), 3.87 (s, 6H), 3.73 (s, 3H), 2.67 (s, 3H); LCMS: (M+1)⁺: 451.06.

### EXAMPLE 74

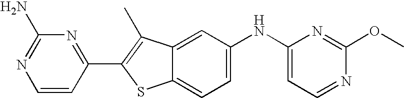

### 4-(5-(2-Methoxypyrimidin-4-ylamino)-3-methylbenzo[b]thiophen-2-yl)pyrimidin-2-amine

Step 1

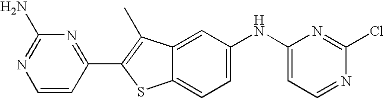

### 4-(5-(2-Chloropyrimidin-4-ylamino)-3-methylbenzo[b]thiophen-2-yl)pyrimidin-2-amine

A 50 mL round bottom flask was charged with 4-(5-amino-3-methylbenzo[b]thiophen-2-yl)pyrimidin-2-amine (0.5 g, 1.95 mmol, prepared in Example 72), 2,4-chloropyrimidine (0.29 g, 1.95 mmol), N,N-diisopropylethylamine (0.25 g, 1.95 mmol), and EtOH (6.5 mL), then heated to 80° C. for 16 h. LCMS analysis showed complete conversion to product. Work-up: after cooling to room temperature, water was added and the solid material was collected by filtration and washed with water. The crude product was recrystallized from hot isopropyl alcohol to give the title compound (0.42 g, 58% yield) as a yellow solid. LCMS: (M+1)⁺: 368.98.
Step 2

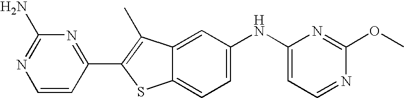

### 4-(5-(2-Methoxypyrimidin-4-ylamino)-3-methylbenzo[b]thiophen-2-yl)pyrimidin-2-amine

A 25 mL round bottom flask was charged with 4-(5-(2-chloropyrimidin-4-ylamino)-3-methylbenzo[b]thiophen-2-yl)pyrimidin-2-amine (0.1 g, 1.95 mmol), THF (1.35 mL) and NaOMe (1.35 mmol, 25% w/w solution in THF). The resulting mixture was heated to reflux overnight. Work-up: after cooling to room temperature, water was added, the mixture was extracted with EtOAc (3×25 mL), and the combined organic phases were washed with water and brine, then dried over Na₂SO₄ and evaporated. The crude material was recrystallized from hot isopropyl alcohol to provide the title compound (0.06 g, 61% yield) as a yellow solid. ¹H NMR (400 MHz, CD₃OD) δ: 8.36 (s, 1H), 8.31 (d, 1H), 8.04-7.99 (m, 2H), 7.67 (d, 1H), 7.25 (d, 1H), 6.67 (d, 1H), 4.02 (s, 3H), 2.81 (s, 3H); LCMS: (M+1)⁺: 364.99.

### EXAMPLE 75

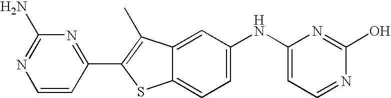

### 4-(2-(2-Aminopyrimidin-4-yl)-3-methylbenzo[b]thiophen-5-ylamino)pyrimidin-2-ol

A 20 mL screw cap vial was charged with 4-(5-(2-methoxypyrimidin-4-ylamino)-3-methylbenzo[b]thiophen-2-yl)pyrimidin-2-amine (0.05 g, 0.14 mmol, prepared in Example 74), and CH₂Cl₂ (1.4 mL), then cooled to −78° C. BBr₃ (0.31 g, 1.23 mmol) was added dropwise and the reaction mixture was allowed to warm to room temperature overnight. Work-up: the reaction mixture was quenched with aqueous NaHCO₃, then extracted with CH₂Cl₂ (3×25 mL). The combined organic phases were washed with water and brine, then dried over Na₂SO₄ and evaporated. The crude material was purified by C18 reverse phase semi-preparative HPLC, giving the title compound (0.009 g, 19% yield) as a yellow solid. LCMS: (M+1)⁺: 351.02.

### EXAMPLE 76

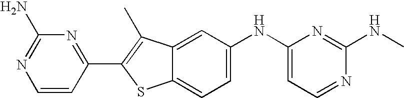

### N⁴-(2-(2-Aminopyrimidin-4-yl)-3-methylbenzo[b]thiophen-5-yl)-N²-methyl pyrimidine-2,4-diamine

A microwave vessel was charged with 4-(5-(2-chloropyrimidin-4-ylamino)-3-methylbenzo[b]thiophen-2-yl)pyrimidin-2-amine (0.1 g, 0.27 mmol, prepared in Example 74, Step 1), methanamine (2.7 mmol) and isopropyl alcohol (1.35 mL) then sealed and irradiated in a microwave at 100° C. for 10 min. Work-up: water was added, the mixture was extracted with EtOAc (3×25 mL) and the combined organic phases were washed with water and brine, then dried over Na₂SO₄ and evaporated. The crude material was purified by C18 reverse phase semi-preparative HPLC, giving the title compound (0.050 g, 51% yield) as a yellow solid. LCMS: (M+1)⁺: 364.01.

### EXAMPLE 77

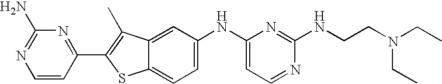

### N⁴-(2-(2-Aminopyrimidin-4-yl)-3-methylbenzo[b]thiophen-5-yl)-N²-(2-(diethylamino) ethyl)pyrimidine-2,4-diamine

The title compound was prepared analogously to N⁴-(2-(2-aminopyrimidin-4-yl)-3-methylbenzo[b]thiophen-5-yl)-N²-methylpyrimidine-2,4-diamine in Example 76, where N¹,N¹-diethylethane-1,2-diamine was substituted for methanamine. LCMS: (M+1)⁺: 449.06.

### EXAMPLE 78

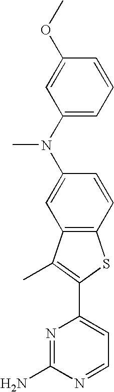

### 4-(5-((3-Methoxyphenyl)(methyl)amino)-3-methylbenzo[b]thiophen-2-yl)pyrimidin-2-amine

An 8 mL pierceable screw cap vial was charged with 4-(5-bromo-3-methylbenzo[b]thiophen-2-yl)pyrimidin-2-amine (128 mg, 0.400 mmol, prepared as described in Example 13), 3-methoxy-N-methylaniline (0.105 mL, 0.803 mmol), tris(dibenzylideneacetone)dipalladium(0) (23 mg, 0.025 mmol), 1,3-bis(2,6-diisopropylphenyl)imidazolium chloride (34 mg, 0.080 mmol), and sodium tert-butoxide (192 mg, 2.00 mmol), then evacuated and back-filled with nitrogen (3×). Dioxane (2 mL, anhydrous) was added and nitrogen was bubbled through the reaction mixture for approx. 10 min. The reaction vessel was sealed and stirred in a 95° C. oil bath for 16 h, then allowed to cool and then filtered through Celite. The filtrate was evaporated and the crude product was purified by silica gel chromatography, eluting with methanol in CH₂Cl₂ and then further purified by C18 reverse phase semi-preparative HPLC, giving the product as an orange solid (mono TFA salt, 1.1 mg, 0.6%.) ¹H NMR (400 MHz, CD₃OD) δ: 8.26 (m, 1H), 7.78 (m, 1H), 7.53 (m, 1H), 7.24 (m, 2H), 7.16 (m, 1H), 6.56 (m, 3H), 3.73 (s, 3H), 3.37 (s, 3H), 2.76 (s, 3H). LCMS (M+1⁺): 377.00.

### EXAMPLE 79

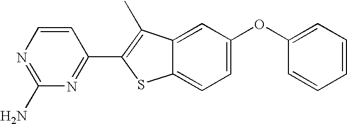

### 4-(3-Methyl-5-phenoxybenzo[b]thiophen-2-yl)pyrimidin-2-amine

Step 1:

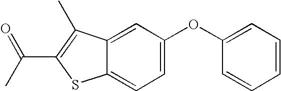

### 1-(3-Methyl-5-phenoxybenzo[b]thiophen-2-yl)ethanone

A 250 mL round bottom flask was charged with 1-(5-bromo-3-methylbenzo[b]thiophen-2-yl)ethanone (1.34 g, 4.98 mmol) prepared as described in Example 12, phenol (470 mg, 4.99 mmol), K₃PO₄ (2.12 g, 9.99 mmol), Pd(OAc)₂ (100 mg, 0.450 mmol), 2-(di-t-butylphosphino)biphenyl (220 mg, 0.740 mmol), in toluene (50 mL). The resulting mixture was stirred at reflux under nitrogen atmosphere for 24 hours, and monitored by TLC (EtOAc/petroleum ether=1/50). The reaction mixture was cooled and filtered. The filtrate was concentrated and purified by eluting through a silica gel column with EtOAc/petroleum ether (1/50) to obtain 0.32 g (23%) of the product as a white solid. ¹H NMR (300 MHz, CDCl₃) δ: 7.79-7.62 (m, 1H), 7.45-7.00 (m, 7H), 2.66 (s, 3H), 2.63 (s, 3H).
Step 2

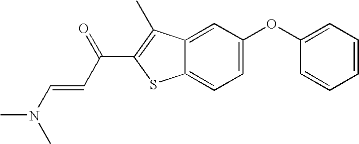

### (E)-3-(Dimethylamino)-1-(3-methyl-5-phenoxybenzo[b]thiophen-2-yl)prop-2-en-1-one

A 10 mL round bottom flask was charged with 1-(3-methyl-5-phenoxybenzo[b]thiophen-2-yl)ethanone (320 mg, 1.13 mmol), and DMFDMA (5 mL). The resulting solution was stirred at reflux for 24 hours. The residue was concentrated to afford the product as a yellow solid (350 mg). The product was used without further purification.
Step 3

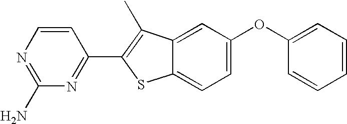

### 4-(3-Methyl-5-phenoxybenzo[b]thiophen-2-yl)pyrimidin-2-amine

A 25 mL round bottom flask was charged with a solution of freshly prepared EtONa (322 mg, 4.13 mmol), guanidine hydrochloride (396 mg, 4.15 mmol), and ethanol (5 mL). The resulting mixture was stirred for 0.5 hours at reflux, then cooled to room temperature and filtered to remove the sodium chloride. To the filtrate was added (E)-3-(dimethylamino)-1-(3-methyl-5-phenoxybenzo[b]thiophen-2-yl)prop-2-en-1-one (350 mg, 1.04 mmol), which was then stirred for 4 hours at reflux. The reaction was monitored by TLC eluted with EtOAc/TEA (1 mL/1 drop). After filtration, the reaction mixture was cooled where a solid formed. The solid was isolated by filtration and washed with ethanol (2 mL). Purification via flash chromatograph eluted with EtOAc afforded the product as a white solid (136 mg, 39%). ¹H NMR (300 MHz, DMSO-d₆) δ: 8.35 (d, 1H), 7.99 (d, 1H), 7.54-6.97 (m, 8H), 6.80 (s, 2H), 2.61 (s, 3H).). LCMS (M+1)⁺: 334.10.

### EXAMPLE 80

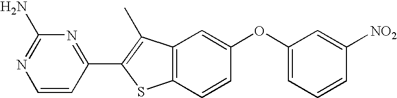

### 4-(3-Methyl-5-(3-nitrophenoxy)benzo[b]thiophen-2-yl)pyrimidin-2-amine

The title compound was prepared analogously to 4-(3-methyl-5-phenoxybenzo[b]thiophen-2-yl)pyrimidin-2-amine, where 3-nitrophenol was substituted for phenol as described in Example 79. ¹H NMR (300 MHz, CDCl₃) δ: 8.36 (m, 1H), 8.10 (m, 1H), 7.98 (m, 1H), 7.68 (m, 3H), 7.52 (m, 1H), 7.28 (m, 1H), 7.00 (m, 1H), 6.81 (s, 2H), 2.64 (s, 3H). LCMS (M+1)⁺: 379.

### EXAMPLE 81

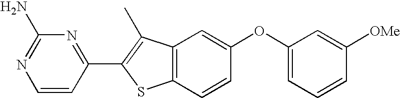

### 4-(5-(3-Methoxyphenoxy)-3-methylbenzo[b]thiophen-2-yl)pyrimidin-2-amine

The title compound was prepared analogously to 4-(3-methyl-5-phenoxybenzo[b]thiophen-2-yl)pyrimidin-2-amine, where 3-methoxyphenol was substituted for phenol as described in Example 79. ¹H NMR (300 MHz, DMSO-d₆) δ: 8.35 (d, 1H), 7.99 (d, 1H), 7.55 (d, 1H), 7.28 (t, 1H), 7.17 (dd, 1H), 6.97 (d, 1H), 6.61 (t, 1H), 6.58-6.52 (m, 1H), 3.74 (s, 3H), 2.62 (s, 3H). LCMS (M+1)⁺: 391.

### EXAMPLE 82

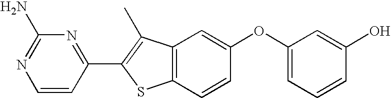

### 3-(2-(2-Aminopyrimidin-4-yl)-3-methylbenzo[b]thiophen-5-yloxy)phenol

A 5 mL round bottom flask was charged with 4-(5-(3-methoxyphenoxy)-3-methylbenzo[b]thiophen-2-yl)pyrimidin-2-amine (15.8 mg, 0.0435 mmol, prepared as described in Example 81), and methylene chloride (0.5 mL). The resulting solution was cooled to −78° C. under a nitrogen atmosphere, where BBr₃ (98.0 mg, 0.391 mmol) was added dropwise. The reaction was stirred overnight at room temperature. Work-up: the mixture was poured over ice water (25 mL), extracted three times with EtOAc (25 mL), washed with brine (50 mL), and dried over Na₂SO₄. The mixture was concentrated, and purified by SiO₂ flash chromatography, eluting with 10% methanol and methylene chloride to afford the title compound in 11.2 mg (74% yield), as an off white solid. ¹H NMR (400 MHz, DMSO-d₆) δ: 9.55 (s, 1H), 8.34 (d, 1H), 7.97 (d, 1H), 7.52 (d, 1H), 7.14 (m, 2H), 6.96 (d, 1H), 6.76 (s, 2H), 6.49 (d, 1H), 6.42 (d, 1H), 6.35 (s, 1H), 2.62 (s, 3H). LCMS (M+1)⁺: 350.01.

### EXAMPLE 83

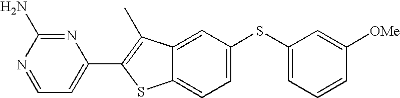

### 4-(5-(3-Methoxyphenylthio)-3-methylbenzo[b]thiophen-2-yl)pyrimidin-2-amine

A 10 mL round bottom flask under nitrogen atmosphere was charged with 4-(5-bromo-3-methylbenzo[b]thiophen-2-yl)pyrimidin-2-amine (96 mg, 0.3 mmol, described in Example 13), 3-methoxybenzenethiol (37 μL, 0.3 mmol), disopropyl ethyl amine (209 μL, 1.2 mmol), Xantphos (17 mg, 0.03 mmol), Pd₂(dba)₃ (13.7 mg, 0.015 mmol), and dioxane (1.0 mL, anhydrous). The resulting mixture was heated in a 98° C. oil bath for 3 hours. Reaction progress was monitored by LCMS. Work-up: the reaction was concentrated, and purified by flash chromatography (gradient elution, 30-50% ethyl acetate/hexanes), giving the title compound as a light yellow powder (102 mg, 90% yield). ¹H NMR (400 MHz, DMSO-d₆) δ 8.35 (d, 1H), 7.99 (m, 2H), 7.40 (d, 1H), 7.25 (t, 1H), 6.98 (d, 1H), 6.79 (m, 4H), 3.69 (s, 3H), 2.64 (s, 3H). LCMS (M+1)⁺: 379.99.

### EXAMPLE 84

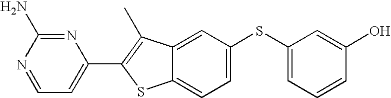

### 3-(2-(2-Aminopyrimidin-4-yl)-3-methylbenzo[b]thiophen-5-ylthio)phenol

A 10 mL round bottom flask under nitrogen atmosphere was charged with 4-(5-(3-methoxyphenylthio)-3-methylbenzo[b]thiophen-2-yl)pyrimidin-2-amine (33 mg, 0.081 mmol, described in Example 83), methylene chloride (0.36 mL), cooled to −78° C., and treated with BBr₃ (31 μL, 0.327 mmol). The resulting mixture was allowed to slowly warm to room temperature and stir overnight. Reaction progress was monitored by LCMS. Work-up: the reaction was diluted with ethyl acetate, washed with NaHCO₃ (1N aq.), concentrated, and purified by C18 semi-preparative reverse phase HPLC. The product was as a light yellow powder (4.3 mg, 33% yield). ¹H NMR (400 MHz, DMSO-d₆) δ 9.55 (bs, 1H), 8.36 (d, 1H), 8.00 (d, 1H), 7.99 (s, 1H), 7.43 (m, 1H), 7.12 (t, 2H), 6.70 (d, 1H), 6.62 (m, 1H), 6.57 (m, 1H), 2.66 (s, 3H). LCMS (M+1)⁺: 366.12.

### EXAMPLE 85

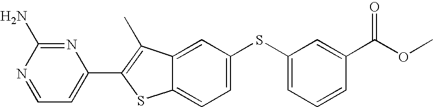

### Methyl 3-(2-(2-aminopyrimidin-4-yl)-3-methylbenzo[b]thiophen-5-ylthio)benzoate

The title compound was prepared analogously to 4-(5-(3-methoxyphenylthio)-3-methylbenzo[b]thiophen-2-yl)pyrimidin-2-amine (Example 83), where methyl 3-mercaptobenzoate was substituted for 3-methoxybenzenethiol in the final step of the sequence. ¹H NMR (400 MHz, CDCl₃) δ 8.39 (d, 1H), 7.98 (s, 1H), 7.91 (s, 1H), 7.87 (d, 1H), 7.85 (d, 1H), 7.42 (m, 2H), 7.32 (t, 1H), 7.00 (d, 1H), 5.11 (bs, 2H), 3.89 (s, 3H), 2.68 (s, 3H). LCMS (M+1)⁺: 408.15.

### EXAMPLE 86

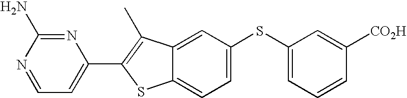

### 3-(2-(2-Aminopyrimidin-4-yl)-3-methylbenzo[b]thiophen-5-ylthio)benzoic acid

A 10 mL round bottom flask containing a solution of NaOH (5.6 mg, 0.243 mmol), water (100 μL), methanol (700 μL), and THF (700 μL) was treated with methyl 3-(2-(2-aminopyrimidin-4-yl)-3-methylbenzo[b]thiophen-5-ylthio)benzoate (33 mg, 0.081 mmol, described in Example 85). The resulting mixture was stirred at room temperature for 3 hours. Reaction progress was monitored by LCMS. Work-up: the reaction was neutralized with citric acid (1M aq.), diluted with water (1 mL), and concentrated until solid formed. The solid was isolated by filtration, rinsed with water and ether, and dried under high vacuum, giving the product as a light yellow powder (22 mg, 69% yield). ¹H NMR (400 MHz, CDCl₃) δ 13.11 (s, 1H), 8.35 (d, 1H), 8.05 (m, 2H), 7.77 (d, 1H), 7.71 (s, 1H), 7.46 (m, 3H), 6.98 (d, 1H), 6.79 (s, 2H), 2.64 (s, 3H). LCMS (M+1)⁺: 394.13.

### EXAMPLE 87

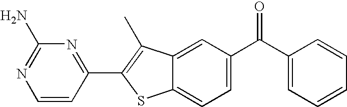

### (2-(2-Aminopyrimidin-4-yl)-3-methylbenzo[b]thiophen-5-yl)(phenyl)methanone

A 25 mL round bottom flask was charged with 2-(2-aminopyrimidin-4-yl)-3-methylbenzo[b]thiophene-5-carbonitrile (0.010 g, 0.037 mmol, prepared in Step 1 of Example 32) and THF, then cooled to 0° C. Phenyllithium (0.148 mmol) was added and the reaction mixture was stirred for 1 h. Work-up: methanol was added and the mixture was partitioned between EtOAc (2×10 mL) and brine. The combined organic phases were dried over Na₂SO₄ and evaporated. The crude product was purified by C18 reverse phase semi-preparative HPLC, giving the title compound (2 mg, 15% yield) as an off-white solid. ¹H NMR (400 MHz, CD₃OD) δ: 8.32-8.31 (m, 2H), 8.07 (dd, 1H), 7.90 (dd, 1H), 7.84-7.82 (m, 2H), 7.70-7.66 (m, 1H), 7.59-7.55 (m, 2H), 7.30 (d, 1H), 2.82 (s, 3H); LCMS: (M+1)⁺: 345.81.

### EXAMPLE 88

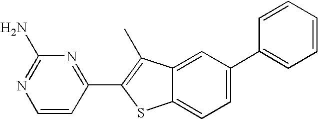

### 4-(3-Methyl-5-phenylbenzo[b]thiophen-2-yl)pyrimidin-2-amine

A microwave vessel was charged with 4-(5-bromo-3-methylbenzo[b]thiophen-2-yl)pyrimidin-2-amine (0.015 g, 0.047 mmol, prepared in Example 13), phenylboronic acid (0.0086 g, 0.07 mmol), Pd(PPh₃)₂Cl₂ (0.003 g, 0.005 mmol), aqueous Na₂CO₃ (2 M, 0.060 mL) and a 3:1 mixture of THF and water (0.47 mL). This mixture was then degassed and back filled with nitrogen three times, and then the vessel was sealed and irradiated in a microwave at 100° C. for 10 min. Reaction progress was monitored by LCMS. Work-up: water (2 mL) was added, the mixture was extracted with EtOAc (2×10 mL) and the combined organic phases were washed with water and brine, then dried over Na₂SO₄ and evaporated. The crude product was purified by C18 reverse phase semi-preparative HPLC, giving the title compound (2 mg, 41% yield) as an off-white solid. LCMS: (M+1)⁺: 317.93.

### EXAMPLE 89

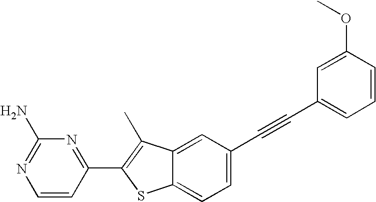

### 4-(5-((3-Methoxyphenyl)ethynyl)-3-methylbenzo[b]thiophen-2-yl)pyrimidin-2-amine

A 10 mL round bottom flask was charged with 4-(5-bromo-3-methylbenzo[b]thiophen-2-yl)pyrimidin-2-amine (0.1 g, 0.31 mmol, prepared in Example 13), Pd(PPh₃)₂Cl₂ (0.022 g, 0.031 mmol), CuI (0.012 g, 0.062 mmol), and THF (1.5 mL). This mixture was degassed three times and back filled with nitrogen, and charged with 1-ethynyl-3-methoxybenzene (0.041 g, 0.31 mmol). The reaction mixture was refluxed overnight, and reaction progress was monitored by LCMS. Work-up: diluted with water (2 mL), extracted with EtOAc (100 mL), washed with brine, dried over Na₂SO₄, and evaporated. The crude product was purified by C18 reverse phase semi-preparative HPLC, giving the title compound (52 mg, 47% yield) as an off-white solid. ¹H NMR (400 MHz, DMSO-d₆) δ: 8.37 (m, 1H), 8.13 (s, 1H), 8.04 (d, 1H), 7.60-7.58 (m, 1H), 7.36-7.32 (m, 1H), 7.16-6.98 (m, 4H), 3.79 (s, 1H), 2.52 (s, 3H); LCMS: (M+1)⁺: 372.00.

### EXAMPLE 90

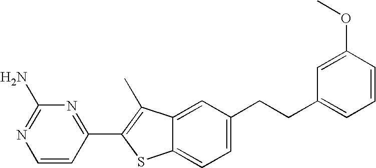

### 4-(5-(3-Methoxyphenethyl)-3-methylbenzo[b]thiophen-2-yl)pyrimidin-2-amine

A 25 mL round bottom flask was charged with 4-(5-((3-methoxyphenyl)ethynyl)-3-methylbenzo[b]thiophen-2-yl)pyrimidin-2-amine (0.02 g, 0.05 mmol, prepared in Example 89), Pd/C (0.006 g, 10% Degussa type), and methanol (5 mL). The reaction mixture was purged with nitrogen, then flushed with hydrogen, and stirred overnight. Work-up: the reaction mixture was filtered through Celite and evaporated. The crude product was purified by C18 reverse phase semi-preparative HPLC, giving the title compound (15 mg, 75% yield) as an off-white solid. LCMS: (M+1)⁺: 380.18.

### EXAMPLE 91

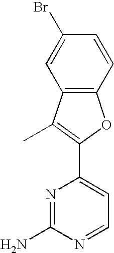

### 4-(5-Bromo-3-methylbenzofuran-2-yl)pyrimidin-2-amine

The title compound was prepared analogously to 4-(5-bromo-3-methylbenzo[b]thiophen-2-yl)pyrimidin-2-amine as described in Example 13, where 4-bromophenol was substituted for 4-bromobenzenethiol in step 1 of that sequence. ¹H NMR (400 MHz, CDCl₃) δ: 8.38 (m, 1H), 7.74 (m, 1H), 7.47 (m, 1H), 7.38 (m, 1H), 7.18 (m, 1H), 5.13 (bs, 2H), 2.68 (s, 3H). LCMS: (M+1)⁺: 304.05.

### EXAMPLE 92

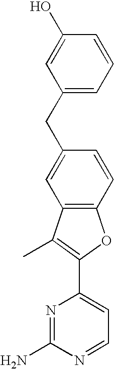

### 3-((2-(2-Aminopyrimidin-4-yl)-3-methylbenzofuran-5-yl)methyl)phenol

Step 1:

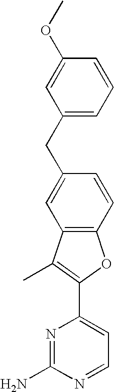

### 4-(5-(3-Methoxybenzyl)-3-methylbenzofuran-2-yl)pyrimidin-2-amine

The title compound was prepared analogously to 4-(5-benzyl-3-methylbenzo[b]thiophen-2-yl)pyrimidin-2-amine as described in Example 24, where (3-methoxybenzyl)zinc (II) chloride was substituted for benzylzinc (II) bromide and 4-(5-bromo-3-methylbenzofuran-2-yl)pyrimidin-2-amine was substituted for 4-(5-bromo-3-methylbenzo[b]thiophen-2-yl)pyrimidin-2-amine. LCMS: (M+1)⁺: 346.20.
Step 2

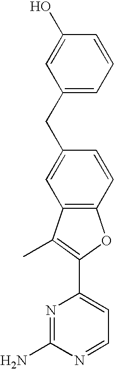

### 3-((2-(2-Aminopyrimidin-4-yl)-3-methylbenzofuran-5-yl)methyl)phenol

The title compound was prepared analogously to 3-(2-(2-aminopyrimidin-4-yl)-3-methylbenzo[b]thiophen-5-yloxy)phenol, where 4-(5-(3-methoxybenzyl)-3-methylbenzofuran-2-yl)pyrimidin-2-amine was substituted for 4-(5-(3-methoxyphenoxy)-3-methylbenzo[b]thiophen-2-yl)pyrimidin-2-amine as described in Example 82. ¹H NMR (400 MHz, DMSO-d₆) δ: 8.35 (m, 1H), 7.58 (m, 1H), 7.54 (m, 1H), 7.30 (m, 1H), 7.08 (m, 2H), 6.68 (m, 1H), 6.60 (m, 1H), 6.56 (m, 1H), 3.96 (s, 2H), 2.69 (s, 3H). LCMS: (M+1)⁺: 332.21.

### EXAMPLE 93

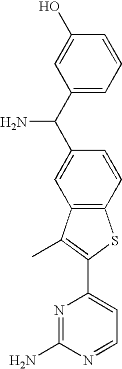

### 3-((2-(2-Aminopyrimidin-4-yl)-3-methylbenzofuran-5-yl)methyl)phenol

4-(5-(Amino(3-methoxyphenyl)methyl)-3-methylbenzo[b]thiophen-2-yl)pyrimidin-2-amine prepared as described in Example 32 was demethylated using BBr₃ as described in Example 82 to give the title compound. ¹H NMR (400 MHz, DMSO-d₆) δ: 9.64 (br, 1H), 8.92 (bm, 3H), 8.36 (m, 1H), 8.09 (m, 1H), 8.03 (m, 1H), 7.45 (m, 1H), 7.23 (m, 1H), 7.02 (m, 1H), 6.93 (m, 1H), 6.83 (m, 1H), 6.74 (m, 1H), 5.72 (m, 1H), 2.70 (s, 3H). LCMS: (M+1)⁺: 363.17.

### EXAMPLE 94

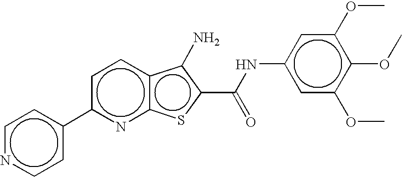

Example 93 is commercially available.

### Compounds Prepared by Parallel Synthesis

The invention is illustrated by the following Schemes:

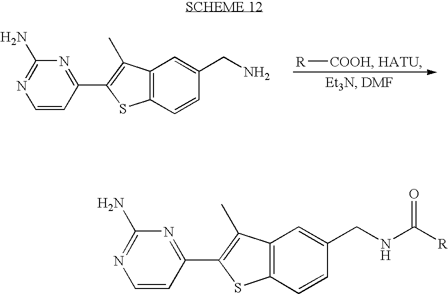

Examples 94-327 can be synthesized using the following general synthetic procedure set forth in Scheme 12.

Starting core: 4-(5-(aminomethyl)-3-methylbenzo[b]thiophen-2-yl)pyrimidin-2-amine was prepared as described in Example 33. Where R—COOH is a carboxylic acid selected to afford Examples 91-324, which were prepared by General Procedure 1.

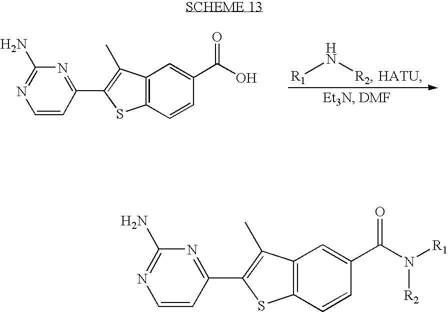

Examples 328-570 can be synthesized using the following general synthetic procedure set forth in Scheme 13.

Starting core: 2-(2-aminopyrimidin-4-yl)-3-methylbenzo[b]thiophene-5-carboxylic acid was prepared as described in Example 19. Where 1° amines, and 2° amines were selected to afford Examples 325-567, which were prepared by General Procedure 2.

General Conditions:

General Conditions 1:

Carboxylic acid monomers (4 μmol) in DMF (8 μL) were transferred to each well of 384 well plate, then treated with a solution of core (1.8 μmol) and Et₃N (6.0 μmol) in DMF (18 μL), followed by a solution HATU (2.0 μmol) in DMF (8 μL). The reaction plate was heat sealed and shaken at room temperature for 16 hours. Solvent was removed under vacuum. Products were analyzed for purity by LCMS before testing.

General Conditions 2:

Amine monomers (4 μmol) in DMF (8 μL) were transferred to each well of a 384 well plate, then treated with a solution of core (4.0 μmol) and Et₃N (8.8 μmol) in DMF (30 μL), followed by a solution HATU (4.4 μmol) in DMF (10 μL). The reaction plate was heat sealed and shaken at room temperature for 16 hours. Solvent was removed under vacuum. Products were analyzed for purity by LCMS before testing.

The invention is further illustrated by the following examples.

| | |
|---|---|
| 95 | CC(═O)NCc1ccc2sc(c(C)c2c1)-c3ccnc(N)n3 |
| 96 | Cc1c(sc2ccc(CNC(═O)C═O)cc12)-c3ccnc(N)n3 |
| 97 | Cc1c(sc2ccc(CNC(═O)CC#N)cc12)-c3ccnc(N)n3 |
| 98 | C[C](O)C(═O)NCc1ccc2sc(c(C)c2c1)-c3ccnc(N)n3 |
| 99 | CCC(═O)C(═O)NCc1ccc2sc(c(C)c2c1)-c3ccnc(N)n3 |
| 100 | Cc1c(sc2ccc(CNC(═O)C(C)(C)C)cc12)-c3ccnc(N)n3 |
| 101 | CC(C)CC(═O)NCc1ccc2sc(c(C)c2c1)-c3ccnc(N)n3 |
| 102 | Cc1c(sc2ccc(CNC(═O)c3cnc[nH]3)cc12)-c4ccnc(N)n4 |
| 103 | Cc1c(sc2ccc(CNC(═O)c3ncc[nH]3)cc12)-c4ccnc(N)n4 |
| 104 | Cc1c(sc2ccc(CNC(═O)C3═CCCC3)cc12)-c4ccnc(N)n4 |
| 105 | Cc1c(sc2ccc(CNC(═O)C(F)(F)F)cc12)-c3ccnc(N)n3 |
| 106 | Cc1c(sc2ccc(CNC(═O)C3CCCO3)cc12)-c4ccnc(N)n4 |
| 107 | CC(═O)NCC(═O)NCc1ccc2sc(c(C)c2c1)-c3ccnc(N)n3 |
| 108 | Cc1c(sc2ccc(CNC(═O)c3ccccc3)cc12)-c4ccnc(N)n4 |
| 109 | Cc1c(sc2ccc(CNC(═O)c3cccnc3)cc12)-c4ccnc(N)n4 |
| 110 | Cc1c(sc2ccc(CNC(═O)c3ccncc3)cc12)-c4ccnc(N)n4 |
| 111 | Cc1c(sc2ccc(CNC(═O)c3ccccn3)cc12)-c4ccnc(N)n4 |
| 112 | Cc1c(sc2ccc(CNC(═O)C(O)CCl)cc12)-c3ccnc(N)n3 |
| 113 | Cc1cc(n[nH]1)C(═O)NCc2ccc3sc(c(C)c3c2)-c4ccnc(N)n4 |
| 114 | Cc1c(sc2ccc(CNC(═O)c3cn(C)cn3)cc12)-c4ccnc(N)n4 |
| 115 | Cc1c(sc2ccc(CNC(═O)C3CCCCC3)cc12)-c4ccnc(N)n4 |
| 116 | Cc1c(sc2ccc(CNC(═O)c3cscn3)cc12)-c4ccnc(N)n4 |
| 117 | CCOC(═O)CC(═O)NCc1ccc2sc(c(C)c2c1)-c3ccnc(N)n3 |
| 118 | Cc1cccc(c1)C(═O)NCc2ccc3sc(c(C)c3c2)-c4ccnc(N)n4 |
| 119 | Cc1ccccc1C(═O)NCc2ccc3sc(c(C)c3c2)-c4ccnc(N)n4 |
| 120 | Cc1ccc(cc1)C(═O)NCc2ccc3sc(c(C)c3c2)-c4ccnc(N)n4 |
| 121 | Cc1c(sc2ccc(CNC(═O)c3cccc(O)c3)cc12)-c4ccnc(N)n4 |
| 122 | Cc1c(sc2ccc(CNC(═O)c3ccccc3O)cc12)-c4ccnc(N)n4 |
| 123 | Cc1c(sc2ccc(CNC(═O)c3ccc(F)cc3)cc12)-c4ccnc(N)n4 |
| 124 | Cc1c(sc2ccc(CNC(═O)c3cccc(F)c3)cc12)-c4ccnc(N)n4 |
| 125 | CCCCCCCC(═O)NCc1ccc2sc(c(C)c2c1)-c3ccnc(N)n3 |
| 126 | Cc1c(sc2ccc(CNC(═O)c3ccc(cc3)C#N)cc12)-c4ccnc(N)n4 |
| 127 | Cc1c(sc2ccc(CNC(═O)c3cccc(c3)C#N)cc12)-c4ccnc(N)n4 |
| 128 | Cc1c(sc2ccc(CNC(═O)C═Cc3ccccc3)cc12)-c4ccnc(N)n4 |
| 129 | Cc1c(sc2ccc(CNC(═O)c3ccccc3C═O)cc12)-c4ccnc(N)n4 |
| 130 | Cc1c(sc2ccc(CNC(═O)c3ccc(C═O)cc3)cc12)-c4ccnc(N)n4 |
| 131 | Cc1ccc(c(C)c1)C(═O)NCc2ccc3sc(c(C)c3c2)-c4ccnc(N)n4 |
| 132 | Cc1ccccc1CC(═O)NCc2ccc3sc(c(C)c3c2)-c4ccnc(N)n4 |
| 133 | Cc1cccc(c1C)C(═O)NCc2ccc3sc(c(C)c3c2)-c4ccnc(N)n4 |
| 134 | Cc1ccc(C)c(c1)C(═O)NCc2ccc3sc(c(C)c3c2)-c4ccnc(N)n4 |
| 135 | Cc1cc(C)cc(c1)C(═O)NCc2ccc3sc(c(C)c3c2)-c4ccnc(N)n4 |
| 136 | Cc1ccc(cc1C)C(═O)NCc2ccc3sc(c(C)c3c2)-c4ccnc(N)n4 |
| 137 | CCc1ccc(cc1)C(═O)NCc2ccc3sc(c(C)c3c2)-c4ccnc(N)n4 |
| 138 | COc1ccc(cc1)C(═O)NCc2ccc3sc(c(C)c3c2)-c4ccnc(N)n4 |
| 139 | Cc1cccc(C(═O)NCc2ccc3sc(c(C)c3c2)-c4ccnc(N)n4)c1O |
| 140 | Cc1c(sc2ccc(CNC(═O)Cc3cccc(O)c3)cc12)-c4ccnc(N)n4 |
| 141 | Cc1ccc(O)c(c1)C(═O)NCc2ccc3sc(c(C)c3c2)-c4ccnc(N)n4 |
| 142 | Cc1c(sc2ccc(CNC(═O)c3cc(O)ccc3O)cc12)-c4ccnc(N)n4 |
| 143 | Cc1c(sc2ccc(CNC(═O)c3cccc(O)c3O)cc12)-c4ccnc(N)n4 |
| 144 | Cc1c(sc2ccc(CNC(═O)c3ccc(O)cc3O)cc12)-c4ccnc(N)n4 |
| 145 | Cc1ccc(cc1F)C(═O)NCc2ccc3sc(c(C)c3c2)-c4ccnc(N)n4 |
| 146 | Cc1ccc(F)cc1C(═O)NCc2ccc3sc(c(C)c3c2)-c4ccnc(N)n4 |
| 147 | Cc1c(sc2ccc(CNC(═O)c3ccccc3Cl)cc12)-c4ccnc(N)n4 |
| 148 | Cc1c(sc2ccc(CNC(═O)c3ccc(Cl)nc3)cc12)-c4ccnc(N)n4 |
| 149 | Cc1c(sc2ccc(CNC(═O)c3cccnc3Cl)cc12)-c4ccnc(N)n4 |
| 150 | Cc1c(sc2ccc(CNC(═O)c3ccnc(Cl)c3)cc12)-c4ccnc(N)n4 |
| 151 | Cc1c(sc2ccc(CNC(═O)c3cc(Cl)ccn3)cc12)-c4ccnc(N)n4 |
| 152 | Cc1c(sc2ccc(CNC(═O)c3ccncc3Cl)cc12)-c4ccnc(N)n4 |
| 153 | Cc1c(sc2ccc(CNC(═O)c3c(F)cccc3F)cc12)-c4ccnc(N)n4 |
| 154 | Cc1c(sc2ccc(CNC(═O)c3cccc(F)c3F)cc12)-c4ccnc(N)n4 |
| 155 | Cc1c(sc2ccc(CNC(═O)c3ccc(F)cc3F)cc12)-c4ccnc(N)n4 |
| 156 | Cc1c(sc2ccc(CNC(═O)c3cc(F)cc(F)c3)cc12)-c4ccnc(N)n4 |
| 157 | Cc1c(sc2ccc(CNC(═O)c3ccc(F)c(F)c3)cc12)-c4ccnc(N)n4 |
| 158 | CC(CC(═O)NCc1ccc2sc(c(C)c2c1)-c3ccnc(N)n3)CC(C)(C)C |
| 159 | Cc1c(sc2ccc(CNC(═O)C3CCc4ccccc34)cc12)-c5ccnc(N)n5 |
| 160 | Cc1c(sc2ccc(CNC(═O)C(Cl)(Cl)Cl)cc12)-c3ccnc(N)n3 |
| 161 | Cc1c(sc2ccc(CNC(═O)C═Cc3ccccc3O)cc12)-c4ccnc(N)n4 |
| 162 | CC(═O)c1ccc(Cc1)C(═O)NCc2ccc3sc(c(C)c3c2)-c4ccnc(N)n4 |
| 163 | Cc1c(sc2ccc(CNC(═O)C═Cc3cccc(O)c3)cc12)-c4ccnc(N)n4 |
| 164 | CC(═O)c1cccc(c1)C(═O)NCc2ccc3sc(c(C)c3c2)-c4ccnc(N)n4 |
| 165 | CC(═O)c1ccccc1C(═O)NCc2ccc3sc(c(C)c3c2)-c4ccnc(N)n4 |
| 166 | CC(C)c1ccc(cc1)C(═O)NCc2ccc3sc(c(C)c3c2)-c4ccnc(N)n4 |
| 167 | Cc1ccccc1CCC(═O)NCc2ccc3sc(c(C)c3c2)-c4ccnc(N)n4 |
| 168 | Cc1cccc(CCC(═O)NCc2ccc3sc(c(C)c3c2)-c4ccnc(N)n4)c1 |
| 169 | CCC(C(═O)NCc1ccc2sc(c(C)c2c1)-c3ccnc(N)n3)c4ccccc4 |
| 170 | Cc1cc(C)c(c(C)c1)C(═O)NCc2ccc3sc(c(C)c3c2)-c4ccnc(N)n4 |
| 171 | CCCc1ccc(cc1)C(═O)NCc2ccc3sc(c(C)c3c2)-c4ccnc(N)n4 |
| 172 | Cc1c(sc2ccc(CNC(═O)c3ccc4OCOc4c3)cc12)-c5ccnc(N)n5 |
| 173 | Cc1c(sc2ccc(CNC(═O)C═Cc3ccccc3F)cc12)-c4ccnc(N)n4 |
| 174 | CCOc1ccccc1C(═O)NCc2ccc3sc(c(C)c3c2)-c4ccnc(N)n4 |
| 175 | Cc1ccccc1OCC(═O)NCc2ccc3sc(c(C)c3c2)-c4ccnc(N)n4 |
| 176 | Cc1c(sc2ccc(CNC(═O)CCc3ccccc3O)cc12)-c4ccnc(N)n4 |
| 177 | COc1ccccc1CC(═O)NCc2ccc3sc(c(C)c3c2)-c4ccnc(N)n4 |
| 178 | CC(C(═O)NCc1ccc2sc(c(C)c2c1)-c3ccnc(N)n3)c4ccc(O)cc4 |
| 179 | Cc1c(sc2ccc(CNC(═O)c3ccccc3[N+]([O−])═O)cc12)-c4ccnc(N)n4 |
| 180 | Cc1c(sc2ccc(CNC(═O)c3cccc(c3)[N+]([O−])═O)cc12)-c4ccnc(N)n4 |
| 181 | COc1cccc(O)c1C(═O)NCc2ccc3sc(c(C)c3c2)-c4ccnc(N)n4 |
| 182 | COc1ccc(O)c(c1)C(═O)NCc2ccc3sc(c(C)c3c2)-c4ccnc(N)n4 |
| 183 | COc1ccc(C(═O)NCc2ccc3sc(c(C)c3c2)-c4ccnc(N)n4)c(O)c1 |
| 184 | COc1ccc(Cc1O)C(═O)NCc2ccc3sc(c(C)c3c2)-c4ccnc(N)n4 |
| 185 | Cc1c(sc2ccc(CNC(═O)c3cc(O)c(O)c(O)c3)cc12)-c4ccnc(N)n4 |
| 186 | Cc1c(sc2ccc(CNC(═O)Cc3ccc(O)c(F)c3)cc12)-c4ccnc(N)n4 |
| 187 | Cc1cc(cc(Cl)n1)C(═O)NCc2ccc3sc(c(C)c3c2)-c4ccnc(N)n4 |
| 188 | Cc1c(sc2ccc(CNC(═O)Cc3c(F)cccc3F)cc12)-c4ccnc(N)n4 |
| 189 | Cc1c(sc2ccc(CNC(═O)c3cccc4ccccc34)cc12)-c5ccnc(N)n5 |
| 190 | Cc1c(sc2ccc(CNC(═O)c3ccc(Cl)cc3O)cc12)-c4ccnc(N)n4 |
| 191 | Cc1c(sc2ccc(CNC(═O)c3cc(Cl)ccc3O)cc12)-c4ccnc(N)n4 |
| 192 | Cc1c(sc2ccc(CNC(═O)c3ccc4ncccc4c3)cc12)-c5ccnc(N)n5 |
| 193 | Cc1c(sc2ccc(CNC(═O)c3cnc(O)c(Cl)c3)cc12)-c4ccnc(N)n4 |
| 194 | Cc1c(sc2ccc(CNC(═O)c3ccc(F)cc3Cl)cc12)-c4ccnc(N)n4 |
| 195 | Cc1c(sc2ccc(CNC(═O)c3c(F)cccc3Cl)cc12)-c4ccnc(N)n4 |
| 196 | Cc1c(sc2ccc(CNC(═O)CNC(═O)OC(C)(C)C)cc12)-c3ccnc(N)n3 |
| 197 | Cc1c(oc2ccccc12)C(═O)NCc3ccc4sc(c(C)c4c3)-c5ccnc(N)n5 |
| 198 | Cc1c(sc2ccc(CNC(═O)CC3Cc4ccccc4C3)cc12)-c5ccnc(N)n5 |
| 199 | Cc1c(sc2ccc(CNC(═O)C3CCc4ccccc4C3)cc12)-c5ccnc(N)n5 |
| 200 | COc1ccccc1C═CC(═O)NCc2ccc3sc(c(C)c3c2)-c4ccnc(N)n4 |
| 201 | Cc1c(sc2ccc(CNC(═O)c3csc4ccccc34)cc12)-c5ccnc(N)n5 |
| 202 | Cc1c(sc2ccc(CNC(═O)c3cc4ccccc4s3)cc12)-c5ccnc(N)n5 |
| 203 | CCCCc1ccc(cc1)C(═O)NCc2ccc3sc(c(C)c3c2)-c4ccnc(N)n4 |
| 204 | Cc1c(sc2ccc(CNC(═O)c3ccc(cc3)C(C)(C)C)cc12)-c4ccnc(N)n4 |
| 205 | CN(C═O)c1ccc(cc1)C(═O)NCc2ccc3sc(c(C)c3c2)-c4ccnc(N)n4 |
| 206 | CC(═O)Nc1cccc(c1)C(═O)NCc2ccc3sc(c(C)c3c2)-c4ccnc(N)n4 |
| 207 | Cc1c(sc2ccc(CNC(═O)COc3ccccc3C═O)cc12)-c4ccnc(N)n4 |
| 208 | CC(═O)Oc1ccc(cc1)C(═O)NCc2ccc3sc(c(C)c3c2)-c4ccnc(N)n4 |
| 209 | COC(═O)c1ccc(cc1)C(═O)NCc2ccc3sc(c(C)c3c2)-c4ccnc(N)n4 |
| 210 | CC(═O)Oc1cccc(c1)C(═O)NCc2ccc3sc(c(C)c3c2)-c4ccnc(N)n4 |
| 211 | CC(═O)Oc1ccccc1C(═O)NCc2ccc3sc(c(C)c3c2)-c4ccnc(N)n4 |
| 212 | Cc1c(sc2ccc(CNC(═O)Cc3ccc4OCOc4c3)cc12)-c5ccnc(N)n5 |
| 213 | CCCOc1ccc(cc1)C(═O)NCc2ccc3sc(c(C)c3c2)-c4ccnc(N)n4 |
| 214 | CC(C)Oc1ccc(cc1)C(═O)NCc2ccc3sc(c(C)c3c2)-c4ccnc(N)n4 |
| 215 | Cc1cccc(c1C(═O)NCc2ccc3sc(c(C)c3c2)-c4ccnc(N)n4)[N+]([O−])═O |
| 216 | Cc1cccc(C(═O)NCc2ccc3sc(c(C)c3c2)-c4ccnc(N)n4)c1[N+]([O−])═O |
| 217 | Cc1ccc(c(c1)C(═O)NCc2ccc3sc(c(C)c3c2)-c4ccnc(N)n4)[N+]([O−])═O |
| 218 | Cc1c(cccc1[N+]([O−])═O)C(═O)NCc2ccc3sc(c(C)c3c2)-c4ccnc(N)n4 |
| 219 | COc1ccc(C(═O)NCc2ccc3sc(c(C)c3c2)-c4ccnc(N)n4)c(OC)c1 |
| 220 | COc1cccc(OC)c1C(═O)NCc2ccc3sc(c(C)c3c2)-c4ccnc(N)n4 |
| 221 | COc1cc(OC)cc(c1)C(═O)NCc2ccc3sc(c(C)c3c2)-c4ccnc(N)n4 |
| 222 | COc1cccc(C(═O)NCc2ccc3sc(c(C)c3c2)-c4ccnc(N)n4)c1OC |
| 223 | Cc1ccc(SCC(═O)NCc2ccc3sc(c(C)c3c2)-c4ccnc(N)n4)cc1 |
| 224 | Cc1c(sc2ccc(CNC(═O)C═Cc3ccc(Cl)cc3)cc12)-c4ccnc(N)n4 |
| 225 | Cc1c(sc2ccc(CNC(═O)C═Cc3ccccc3Cl)cc12)-c4ccnc(N)n4 |
| 226 | Cc1c(sc2ccc(CNC(═O)c3ccc(c(O)c3)[N+]([O−])═O)cc12)-c4ccnc(N)n4 |
| 227 | Cc1c(sc2ccc(CNC(═O)c3cc(O)ccc3[N+]([O−])═O)cc12)-c4ccnc(N)n4 |
| 228 | Cc1c(sc2ccc(CNC(═O)c3cc(ccc3O)[N+]([O−])═O)cc12)-c4ccnc(N)n4 |
| 229 | Cc1c(sc2ccc(CNC(═O)c3cc(ccc3F)[N+]([O−])═O)cc12)-c4ccnc(N)n4 |
| 230 | Cc1c(sc2ccc(CNC(═O)c3cc(F)ccc3[N+]([O−])═O)cc12)-c4ccnc(N)n4 |
| 231 | Cc1c(sc2ccc(CNC(═O)c3ccc(F)c(c3)[N+]([O−])═O)cc12)-c4ccnc(N)n4 |
| 232 | COc1ccc(Cl)cc1C(═O)NCc2ccc3sc(c(C)c3c2)-c4ccnc(N)n4 |
| 233 | COc1cc(Cl)ccc1C(═O)NCc2ccc3sc(c(C)c3c2)-c4ccnc(N)n4 |
| 234 | Cc1c(sc2ccc(CNC(═O)Cc3ccc(O)c(Cl)c3)cc12)-c4ccnc(N)n4 |
| 235 | COc1cc(cc(Cl)n1)C(═O)NCc2ccc3sc(c(C)c3c2)-c4ccnc(N)n4 |
| 236 | Cc1c(sc2ccc(CNC(═O)c3ccc4cc(O)ccc4c3)cc12)-c5ccnc(N)n5 |
| 237 | Cc1c(sc2ccc(CNC(═O)c3c(O)ccc4ccccc34)cc12)-c5ccnc(N)n5 |
| 238 | Cc1c(sc2ccc(CNC(═O)c3cc4ccccc4cc3O)cc12)-c5ccnc(N)n5 |
| 239 | Cc1c(sc2ccc(CNC(═O)c3ccc4ccccc4c3O)cc12)-c5ccnc(N)n5 |
| 240 | Cc1c(sc2ccc(CNC(═O)c3ccc4cccc(O)c4n3)cc12)-c5ccnc(N)n5 |
| 241 | C[C](NC(═O)OC(C)(C)C)C(═O)NCc1ccc2sc(c(C)c2c1)-c3ccnc(N)n3 |
| 242 | C[C](NC(═O)OC(C)(C)C)C(═O)NCc1ccc2sc(c(C)c2c1)-c3ccnc(N)n3 |
| 243 | Cc1c(sc2ccc(CNC(═O)c3ccccc3C(F)(F)F)cc12)-c4ccnc(N)n4 |
| 244 | Cc1c(sc2ccc(CNC(═O)CCc3nc4ccccc4[nH]3)cc12)-c5ccnc(N)n5 |
| 245 | Cc1c(sc2ccc(CNC(═O)c3cccc(Cl)c3Cl)cc12)-c4ccnc(N)n4 |
| 246 | Cc1c(sc2ccc(CNC(═O)c3ccc(Cl)cc3Cl)cc12)-c4ccnc(N)n4 |
| 247 | Cc1c(sc2ccc(CNC(═O)c3cc(Cl)ccc3Cl)cc12)-c4ccnc(N)n4 |
| 248 | Cc1c(sc2ccc(CNC(═O)c3cc(F)c(F)c(O)c3F)cc12)-c4ccnc(N)n4 |
| 249 | Cc1c(sc2ccc(CNC(═O)C═Cc3ccc4OCOc4c3)cc12)-c5ccnc(N)n5 |
| 250 | Cc1ccc(cc1)C(═O)CCC(═O)NCc2ccc3sc(c(C)c3c2)-c4ccnc(N)n4 |
| 251 | CCCCCc1ccc(cc1)C(═O)NCc2ccc3sc(c(C)c3c2)-c4ccnc(N)n4 |
| 252 | Cc1c(sc2ccc(CNC(═O)c3cc(F)c(F)cc3Cl)cc12)-c4ccnc(N)n4 |
| 253 | Cc1c(sc2ccc(CNC(═O)c3cc(F)c(Cl)cc3F)cc12)-c4ccnc(N)n4 |
| 254 | CCN(CC)c1ccc(cc1)C(═O)NCc2ccc3sc(c(C)c3c2)-c4ccnc(N)n4 |
| 255 | COc1cc(C═CC(═O)NCc2ccc3sc(c(C)c3c2)-c4ccnc(N)n4)ccc1O |
| 256 | COc1ccc(C═CC(═O)NCc2ccc3sc(c(C)c3c2)-c4ccnc(N)n4)cc1O |
| 257 | CCCCOc1ccc(cc1)C(═O)NCc2ccc3sc(c(C)c3c2)-c4ccnc(N)n4 |
| 258 | Cc1c(sc2ccc(CNC(═O)c3cc4cc(Cl)ccc4o3)cc12)-c5ccnc(N)n5 |
| 259 | COc1ccc(c(c1)C(═O)NCc2ccc3sc(c(C)c3c2)-c4ccnc(N)n4)[N+]([O−])═O |
| 260 | COc1cc(cc(OC)c1O)C(═O)NCc2ccc3sc(c(C)c3c2)-c4ccnc(N)n4 |
| 261 | Cc1c(sc2ccc(CNC(═O)c3ccccc3-c4ccccc4)cc12)-c5ccnc(N)n5 |
| 262 | Cc1c(sc2ccc(CNC(═O)c3cccc(Br)c3)cc12)-c4ccnc(N)n4 |
| 263 | Cc1c(sc2ccc(CNC(═O)c3ccc(Br)cc3)cc12)-c4ccnc(N)n4 |
| 264 | Cc1c(sc2ccc(CNC(═O)c3cccc(Cl)c3[N+]([O−])═O)cc12)-c4ccnc(N)n4 |
| 265 | Cc1c(sc2ccc(CNC(═O)c3cc(ccc3Cl)[N+]([O−])═O)cc12)-c4ccnc(N)n4 |
| 266 | Cc1c(sc2ccc(CNC(═O)c3ccc(cc3Cl)[N+]([O−])═O)cc12)-c4ccnc(N)n4 |
| 267 | Cc1c(sc2ccc(CNC(═O)c3ccc(Cl)cc3[N+]([O−])═O)cc12)-c4ccnc(N)n4 |
| 268 | Cc1c(sc2ccc(CNC(═O)c3ccc(Cl)c(c3)[N+]([O−])═O)cc12)-c4ccnc(N)n4 |
| 269 | Cc1c(sc2ccc(CNC(═O)c3cc(Cl)ccc3[N+]([O−])═O)cc12)-c4ccnc(N)n4 |
| 270 | Cc1c(sc2ccc(CNC(═O)c3cccc(Br)n3)cc12)-c4ccnc(N)n4 |
| 271 | Cc1c(sc2ccc(CNC(═O)c3cncc(Br)c3)cc12)-c4ccnc(N)n4 |
| 272 | Cc1c(sc2ccc(CNC(═O)c3cc(F)c(F)cc3[N+]([O−])═O)cc12)-c4ccnc(N)n4 |
| 273 | Cc1c(sc2ccc(CNC(═O)Cc3ccc(cc3)C(F)(F)F)cc12)-c4ccnc(N)n4 |
| 274 | CC(C)Cc1ccc(cc1)C(C)C(═O)NCc2ccc3sc(c(C)c3c2)-c4ccnc(N)n4 |
| 275 | Cc1c(sc2ccc(CNC(═O)c3cc(Cl)c(O)c(Cl)c3)cc12)-c4ccnc(N)n4 |
| 276 | Cc1c(sc2ccc(CNC(═O)c3c(F)c(F)c(F)c(F)c3F)cc12)-c4ccnc(N)n4 |
| 277 | COc1cc(OC)c(C(═O)NCc2ccc3sc(c(C)c3c2)-c4ccnc(N)n4)c(OC)c1 |
| 278 | COc1cc(OC)c(cc1OC)C(═O)NCc2ccc3sc(c(C)c3c2)-c4ccnc(N)n4 |
| 279 | COc1cc(cc(OC)c1OC)C(═O)NCc2ccc3sc(c(C)c3c2)-c4ccnc(N)n4 |
| 280 | COc1ccc(C(═O)NCc2ccc3sc(c(C)c3c2)-c4ccnc(N)n4)c(OC)c1OC |
| 281 | Cc1c(sc2ccc(CNC(═O)c3ccccc3Cc4ccccc4)cc12)-c5ccnc(N)n5 |
| 282 | Cc1c(sc2ccc(CNC(═O)c3cccc(Oc4ccccc4)c3)cc12)-c5ccnc(N)n5 |
| 283 | Cc1c(sc2ccc(CNC(═O)c3ccccc3Oc4ccccc4)cc12)-c5ccnc(N)n5 |
| 284 | Cc1c(sc2ccc(CNC(═O)c3ccc(Oc4ccccc4)cc3)cc12)-c5ccnc(N)n5 |
| 285 | Cc1c(sc2ccc(CNC(═O)c3ccc(cc3)-c4ccc(O)cc4)cc12)-c5ccnc(N)n5 |
| 286 | Cc1cc(ccc1Br)C(═O)NCc2ccc3sc(c(C)c3c2)-c4ccnc(N)n4 |
| 287 | Cc1ccc(cc1Br)C(═O)NCc2ccc3sc(c(C)c3c2)-c4ccnc(N)n4 |
| 288 | Cc1c(sc2ccc(CNC(═O)Cc3ccc(Br)cc3)cc12)-c4ccnc(N)n4 |
| 289 | Cc1c(sc2ccc(CNC(═O)[C]3CCCN3C(═O)OC(C)(C)C)cc12)-c4ccnc(N)n4 |
| 290 | Cc1c(sc2ccc(CNC(═O)C═Cc3ccccc3C(F)(F)F)cc12)-c4ccnc(N)n4 |
| 291 | CCOc1ccc2ccccc2c1C(═O)NCc3ccc4sc(c(C)c4c3)-c5ccnc(N)n5 |
| 292 | CC(C)[C](NC(═O)OC(C)(C)C)C(═O)NCc1ccc2sc(c(C)c2c1)-c3ccnc(N)n3 |
| 293 | CC(C)[C](NC(═O)OC(C)(C)C)C(═O)NCc1ccc2sc(c(C)c2c1)-c3ccnc(N)n3 |
| 294 | Cc1c(sc2ccc(CNC(═O)CCc3cccc(c3)C(F)(F)F)cc12)-c4ccnc(N)n4 |
| 295 | CC1═NN(C(═O)C1)c2ccc(cc2)C(═O)NCc3ccc4sc(c(C)c4c3)-c5ccnc(N)n5 |
| 296 | Cc1nc(Br)sc1C(═O)NCc2ccc3sc(c(C)c3c2)-c4ccnc(N)n4 |
| 297 | Cc1c(sc2ccc(CNC(═O)c3c4ccccc4cc5ccccc35)cc12)-c6ccnc(N)n6 |
| 298 | CC(C)c1cc(C(C)C)c(O)c(c1)C(═O)NCc2ccc3sc(c(C)c3c2)-c4ccnc(N)n4 |
| 299 | CC(═O)N[C](Cc1ccc(O)cc1)C(═O)NCc2ccc3sc(c(C)c3c2)-c4ccnc(N)n4 |
| 300 | Cc1c(sc2ccc(CNC(═O)c3c(Cl)cc(Cl)cc3Cl)cc12)-c4ccnc(N)n4 |
| 301 | COc1cc(C(═O)NCc2ccc3sc(c(C)c3c2)-c4ccnc(N)n4)c(cc1OC)[N+]([O−])═O |
| 302 | Cc1c(sc2ccc(CNC(═O)c3ccc(OCc4ccccc4)cc3)cc12)-c5ccnc(N)n5 |
| 303 | Cc1c(sc2ccc(CNC(═O)CCc3ccccc3Br)cc12)-c4ccnc(N)n4 |
| 304 | Cc1c(sc2ccc(CNC(═O)CCc3cccc(Br)c3)cc12)-c4ccnc(N)n4 |
| 305 | Cc1c(sc2ccc(CNC(═O)[C]3CCCCN3C(═O)OC(C)(C)C)cc12)-c4ccnc(N)n4 |
| 306 | Cc1c(sc2ccc(CNC(═O)c3ccc(Oc4cccc(O)c4)cc3)cc12)-c5ccnc(N)n5 |
| 307 | COc1ccc2cc(ccc2c1)[C](C)C(═O)NCc3ccc4sc(c(C)c4c3)-c5ccnc(N)n5 |
| 308 | Cc1c(sc2ccc(CNC(═O)[C]3CN(CCN3)C(═O)OC(C)(C)C)cc12)-c4ccnc(N)n4 |
| 309 | Cc1c(sc2ccc(CNC(═O)[C]3CN(CCN3)C(═O)OC(C)(C)C)cc12)-c4ccnc(N)n4 |
| 310 | COc1ccc(Br)c(c1)C(═O)NCc2ccc3sc(c(C)c3c2)-c4ccnc(N)n4 |
| 311 | Cc1c(sc2ccc(CNC(═O)[C]3CC(O)CN3C(═O)OC(C)(C)C)cc12)-c4ccnc(N)n4 |
| 312 | CC(C)C(N(C)C(═O)OC(C)(C)C)C(═O)NCc1ccc2sc(c(C)c2c1)-c3ccnc(N)n3 |
| 313 | Cc1c(sc2ccc(CNC(═O)c3ccc(cc3[N+]([O−])═O)C(F)(F)F)cc12)-c4ccnc(N)n4 |
| 314 | Cc1c(sc2ccc(CNC(═O)c3cc(Br)ccc3Cl)cc12)-c4ccnc(N)n4 |
| 315 | Cc1c(sc2ccc(CNC(═O)c3ccc(Cl)c(Br)c3)cc12)-c4ccnc(N)n4 |
| 316 | Cc1c(sc2ccc(CNC(═O)c3ccccc3C(═O)c4ccc(O)cc4)cc12)-c5ccnc(N)n5 |
| 317 | Cc1c(sc2ccc(CNC(═O)c3ccccc3C(═O)c4ccc(F)cc4)cc12)-c5ccnc(N)n5 |
| 318 | CC(C(═O)NCc1ccc2sc(c(C)c2c1)-c3ccnc(N)n3)c4ccc(c(F)c4)-c5ccccc5 |
| 319 | Cc1c(sc2ccc(CNC(═O)c3ccc(I)cc3)cc12)-c4ccnc(N)n4 |
| 320 | Cc1c(sc2ccc(CNC(═O)c3cccc(I)c3)cc12)-c4ccnc(N)n4 |
| 321 | Cc1c(sc2ccc(CNC(═O)[C]3CCCN3C(═O)OCc4ccccc4)cc12)-c5ccnc(N)n5 |
| 322 | CCC(C)[C](NC(═O)OC(C)(C)C)C(═O)NCc1ccc2sc(c(C)c2c1)-c3ccnc(N)n3 |
| 323 | CC(C)C[C](NC(═O)OC(C)(C)C)C(═O)NCc1ccc2sc(c(C)c2c1)-c3ccnc(N)n3 |
| 324 | CC(C(═O)NCc1ccc2sc(c(C)c2c1)-c3ccnc(N)n3)c4cccc(c4)C(═O)c5ccccc5 |
| 325 | Cc1c(sc2ccc(CNC(═O)[C](Cc3ccccc3)NC(═O)OC(C)(C)C)cc12)-c4ccnc(N)n4 |
| 326 | Cc1c(sc2ccc(CNC(═O)[C](Cc3ccccn3)NC(═O)OC(C)(C)C)cc12)-c4ccnc(N)n4 |
| 327 | Cc1c(sc2ccc(CNC(═O)[C]3Cc4ccccc4CN3C(═O)OC(C)(C)C)cc12)- |
| | c5ccnc(N)n5 |
| 328 | Cc1c(sc2ccc(CNC(═O)C(Cc3ccc(O)cc3)NC(═O)OC(C)(C)C)cc12)c4ccnc(N)n4 |
| 329 | CNC(═O)c1ccc2sc(c(C)c2c1)-c3ccnc(N)n3 |
| 330 | CCNC(═O)c1ccc2sc(c(C)c2c1)-c3ccnc(N)n3 |
| 331 | Cc1c(sc2ccc(cc12)C(═O)NCC#C)-c3ccnc(N)n3 |
| 332 | Cc1c(sc2ccc(cc12)C(═O)NCC#N)-c3ccnc(N)n3 |
| 333 | Cc1c(sc2ccc(cc12)C(═O)NC3CC3)-c4ccnc(N)n4 |
| 334 | CC(C)NC(═O)c1ccc2sc(c(C)c2c1)-c3ccnc(N)n3 |
| 335 | CCCNC(═O)c1ccc2sc(c(C)c2c1)-c3ccnc(N)n3 |
| 336 | Cc1c(sc2ccc(cc12)C(═O)NCCN)-c3ccnc(N)n3 |
| 337 | Cc1c(sc2ccc(cc12)C(═O)NCCO)-c3ccnc(N)n3 |
| 338 | Cc1c(sc2ccc(cc12)C(═O)NCCC#N)-c3ccnc(N)n3 |
| 339 | Cc1c(sc2ccc(cc12)C(═O)NC3CCC3)-c4ccnc(N)n4 |
| 340 | Cc1c(sc2ccc(cc12)C(═O)NCC3CC3)-c4ccnc(N)n4 |
| 341 | CCC(C)NC(═O)c1ccc2sc(c(C)c2c1)-c3ccnc(N)n3 |
| 342 | CC(C)CNC(═O)c1ccc2sc(c(C)c2c1)-c3ccnc(N)n3 |
| 343 | Cc1c(sc2ccc(cc12)C(═O)NCCCO)-c3ccnc(N)n3 |
| 344 | CC(CO)NC(═O)c1ccc2sc(c(C)c2c1)-c3ccnc(N)n3 |
| 345 | COCCNC(═O)c1ccc2sc(c(C)c2c1)-c3ccnc(N)n3 |
| 346 | Cc1c(sc2ccc(cc12)C(═O)NC3CCCC3)-c4ccnc(N)n4 |
| 347 | CCCC(C)NC(═O)c1ccc2sc(c(C)c2c1)-c3ccnc(N)n3 |
| 348 | CCC(C)CNC(═O)c1ccc2sc(c(C)c2c1)-c3ccnc(N)n3 |
| 349 | CC(C)C(C)NC(═O)c1ccc2sc(c(C)c2c1)-c3ccnc(N)n3 |
| 350 | CC(C)CCNC(═O)c1ccc2sc(c(C)c2c1)-c3ccnc(N)n3 |
| 351 | CN(C)CCNC(═O)c1ccc2sc(c(C)c2c1)-c3ccnc(N)n3 |
| 352 | COCC(C)NC(═O)c1ccc2sc(c(C)c2c1)-c3ccnc(N)n3 |
| 353 | CCC(CO)NC(═O)c1ccc2sc(c(C)c2c1)-c3ccnc(N)n3 |
| 354 | Cc1c(sc2ccc(cc12)C(═O)NCCCCO)-c3ccnc(N)n3 |
| 355 | Cc1c(sc2ccc(cc12)C(═O)NCc3ccco3)-c4ccnc(N)n4 |
| 356 | Cc1c(sc2ccc(cc12)C(═O)NC3CCCCC3)-c4ccnc(N)n4 |
| 357 | Cc1c(sc2ccc(cc12)C(═O)NCC3CCCO3)-c4ccnc(N)n4 |
| 358 | Cc1c(sc2ccc(cc12)C(═O)NCCC(C)(C)C)-c3ccnc(N)n3 |
| 359 | CC(C)CC(C)NC(═O)c1ccc2sc(c(C)c2c1)-c3ccnc(N)n3 |
| 360 | Cc1c(sc2ccc(cc12)C(═O)NC3CONC3═O)-c4ccnc(N)n4 |
| 361 | Cc1c(sc2ccc(cc12)C(═O)NN3CCOCC3)-c4ccnc(N)n4 |
| 362 | CC(C)C(CO)NC(═O)c1ccc2sc(c(C)c2c1)-c3ccnc(N)n3 |
| 363 | Cc1c(sc2ccc(cc12)C(═O)NCc3ccccc3)-c4ccnc(N)n4 |
| 364 | Cc1c(sc2ccc(cc12)C(═O)NCc3cccnc3)-c4ccnc(N)n4 |
| 365 | Cc1c(sc2ccc(cc12)C(═O)NCc3ccccn3)-c4ccnc(N)n4 |
| 366 | Cc1c(sc2ccc(cc12)C(═O)NCc3ccncc3)-c4ccnc(N)n4 |
| 367 | Cc1c(sc2ccc(cc12)C(═O)NCc3cccs3)-c4ccnc(N)n4 |
| 368 | CC1CCC(CC1)NC(═O)c2ccc3sc(c(C)c3c2)-c4ccnc(N)n4 |
| 369 | Cc1c(sc2ccc(cc12)C(═O)NCC3CCCCC3)-c4ccnc(N)n4 |
| 370 | Cc1c(sc2ccc(cc12)C(═O)NC3CCCCCC3)-c4ccnc(N)n4 |
| 371 | CC1CCCCC1NC(═O)c2ccc3sc(c(C)c3c2)-c4ccnc(N)n4 |
| 372 | Cc1c(sc2ccc(cc12)C(═O)NCCN3CCCC3)-c4ccnc(N)n4 |
| 373 | CN1CCN(CC1)NC(═O)c2ccc3sc(c(C)c3c2)-c4ccnc(N)n4 |
| 374 | CCN(CC)CCNC(═O)c1ccc2sc(c(C)c2c1)-c3ccnc(N)n3 |
| 375 | Cc1cccc(CNC(═O)c2ccc3sc(c(C)c3c2)-c4ccnc(N)n4)c1 |
| 376 | C[C@H](NC(═O)c1ccc2sc(c(C)c2c1)-c3ccnc(N)n3)c4ccccc4 |
| 377 | C[C@@H](NC(═O)c1ccc2sc(c(C)c2c1)-c3ccnc(N)n3)c4ccccc4 |
| 378 | Cc1c(sc2ccc(cc12)C(═O)NCCc3ccccc3)-c4ccnc(N)n4 |
| 379 | Cc1ccccc1CNC(═O)c2ccc3sc(c(C)c3c2)-c4ccnc(N)n4 |
| 380 | Cc1ccc(CNC(═O)c2ccc3sc(c(C)c3c2)-c4ccnc(N)n4)cc1 |
| 381 | Cc1cnc(CNC(═O)c2ccc3sc(c(C)c3c2)-c4ccnc(N)n4)cn1 |
| 382 | Cc1c(sc2ccc(cc12)C(═O)NCc3cccc(F)c3)-c4ccnc(N)n4 |
| 383 | Cc1c(sc2ccc(cc12)C(═O)NCc3ccc(F)cc3)-c4ccnc(N)n4 |
| 384 | Cc1c(sc2ccc(cc12)C(═O)NCc3ccccc3F)-c4ccnc(N)n4 |
| 385 | Cc1c(sc2ccc(cc12)C(═O)NCCCn3ccnc3)-c4ccnc(N)n4 |
| 386 | CN1CCCC1CCNC(═O)c2ccc3sc(c(C)c3c2)-c4ccnc(N)n4 |
| 387 | Cc1c(sc2ccc(cc12)C(═O)NCCN3CCCCC3)-c4ccnc(N)n4 |
| 388 | CC(C)CCCC(C)NC(═O)c1ccc2sc(c(C)c2c1)-c3ccnc(N)n3 |
| 389 | Cc1c(sc2ccc(cc12)C(═O)NCCN3CCOCC3)-c4ccnc(N)n4 |
| 390 | CCOC(═O)CC(C)NC(═O)c1ccc2sc(c(C)c2c1)-c3ccnc(N)n3 |
| 391 | Cc1c(sc2ccc(cc12)C(═O)NC3CCc4ccccc34)-c5ccnc(N)n5 |
| 392 | Cc1ccc(CCNC(═O)c2ccc3sc(c(C)c3c2)-c4ccnc(N)n4)cc1 |
| 393 | Cc1ccc(C)c(CNC(═O)c2ccc3sc(c(C)c3c2)-c4ccnc(N)n4)c1 |
| 394 | C[C@H](NC(═O)c1ccc2sc(c(C)c2c1)-c3ccnc(N)n3)c4ccc(C)cc4 |
| 395 | C[C@@H](NC(═O)c1ccc2sc(c(C)c2c1)-c3ccnc(N)n3)c4ccc(C)cc4 |
| 396 | CC(CNC(═O)c1ccc2sc(c(C)c2c1)-c3ccnc(N)n3)c4ccccc4 |
| 397 | Cc1c(sc2ccc(cc12)C(═O)NCCCc3ccccc3)-c4ccnc(N)n4 |
| 398 | Cc1ccc(CNC(═O)c2ccc3sc(c(C)c3c2)-c4ccnc(N)n4)cc1C |
| 399 | CC(Cc1ccncc1)NC(═O)c2ccc3sc(c(C)c3c2)-c4ccnc(N)n4 |
| 400 | Cc1c(sc2ccc(cc12)C(═O)NCCc3ccc(O)cc3)-c4ccnc(N)n4 |
| 401 | Cc1c(sc2ccc(cc12)C(═O)NCCOc3ccccc3)-c4ccnc(N)n4 |
| 402 | COc1ccccc1CNC(═O)c2ccc3sc(c(C)c3c2)-c4ccnc(N)n4 |
| 403 | COc1ccc(CNC(═O)c2ccc3sc(c(C)c3c2)-c4ccnc(N)n4)cc1 |
| 404 | COc1cccc(CNC(═O)c2ccc3sc(c(C)c3c2)-c4ccnc(N)n4)c1 |
| 405 | Cc1c(sc2ccc(cc12)C(═O)NCCc3ccc(F)cc3)-c4ccnc(N)n4 |
| 406 | Cc1c(sc2ccc(cc12)C(═O)NCCc3ccccc3F)-c4ccnc(N)n4 |
| 407 | Cc1c(sc2ccc(cc12)C(═O)NCCc3cccc(F)c3)-c4ccnc(N)n4 |
| 408 | Cc1c(sc2ccc(cc12)C(═O)NCc3cccc(Cl)c3)-c4ccnc(N)n4 |
| 409 | Cc1c(sc2ccc(cc12)C(═O)NCc3ccccc3Cl)-c4ccnc(N)n4 |
| 410 | Cc1c(sc2ccc(cc12)C(═O)NCc3ccc(Cl)cc3)-c4ccnc(N)n4 |
| 411 | Cc1c(sc2ccc(cc12)C(═O)NCCCN3CCCC3═O)-c4ccnc(N)n4 |
| 412 | Cc1c(sc2ccc(cc12)C(═O)NCc3ccc(F)cc3F)-c4ccnc(N)n4 |
| 413 | Cc1c(sc2ccc(cc12)C(═O)NCc3ccc(F)c(F)c3)-c4ccnc(N)n4 |
| 414 | Cc1c(sc2ccc(cc12)C(═O)NCc3cc(F)cc(F)c3)-c4ccnc(N)n4 |
| 415 | Cc1c(sc2ccc(cc12)C(═O)NCCCN3CCOCC3)-c4ccnc(N)n4 |
| 416 | CC(C)N(CCNC(═O)c1ccc2sc(c(C)c2c1)-c3ccnc(N)n3)C(C)C |
| 417 | Cc1c(sc2ccc(cc12)C(═O)NC3CCCc4ccccc34)-c5ccnc(N)n5 |
| 418 | CC(CCc1ccccc1)NC(═O)c2ccc3sc(c(C)c3c2)-c4ccnc(N)n4 |
| 419 | CC(C)c1ccc(CNC(═O)c2ccc3sc(c(C)c3c2)-c4ccnc(N)n4)cc1 |
| 420 | Cc1c(sc2ccc(cc12)C(═O)NCc3ccc4OCOc4c3)-c5ccnc(N)n5 |
| 421 | COc1cccc(CCNC(═O)c2ccc3sc(c(C)c3c2)-c4ccnc(N)n4)c1 |
| 422 | Cc1c(sc2ccc(cc12)C(═O)N[C@H](CO)Cc3ccccc3)-c4ccnc(N)n4 |
| 423 | COc1ccc(CCNC(═O)c2ccc3sc(c(C)c3c2)-c4ccnc(N)n4)cc1 |
| 424 | COc1ccccc1CCNC(═O)c2ccc3sc(c(C)c3c2)-c4ccnc(N)n4 |
| 425 | CCOc1ccccc1CNC(═O)c2ccc3sc(c(C)c3c2)-c4ccnc(N)n4 |
| 426 | COc1ccc(NC(═O)c2ccc3sc(c(C)c3c2)-c4ccnc(N)n4)cc1OC |
| 427 | Cc1c(sc2ccc(cc12)C(═O)NCCc3ccccc3Cl)-c4ccnc(N)n4 |
| 428 | Cc1c(sc2ccc(cc12)C(═O)NCCc3ccc(Cl)cc3)-c4ccnc(N)n4 |
| 429 | Cc1c(sc2ccc(cc12)C(═O)NCCc3cccc(Cl)c3)-c4ccnc(N)n4 |
| 430 | Cc1c(sc2ccc(cc12)C(═O)NC3CC(C)(C)NC(C)(C)C3)-c4ccnc(N)n4 |
| 431 | CCN(CC)CCCC(C)NC(═O)c1ccc2sc(c(C)c2c1)-c3ccnc(N)n3 |
| 432 | Cc1c(sc2ccc(cc12)C(═O)NCc3ccc(F)c(Cl)c3)-c4ccnc(N)n4 |
| 433 | Cc1c(sc2ccc(cc12)C(═O)N(CCC#N)Cc3ccccc3)-c4ccnc(N)n4 |
| 434 | Cc1c(sc2ccc(cc12)C(═O)NCCNC(═O)OC(C)(C)C)-c3ccnc(N)n3 |
| 435 | Cc1c(sc2ccc(cc12)C(═O)NCCc3c[nH]c4ccccc34)-c5ccnc(N)n5 |
| 436 | Cc1c(sc2ccc(cc12)C(═O)NCc3ccc(cc3)C(C)(C)C)-c4ccnc(N)n4 |
| 437 | CN(CCCNC(═O)c1ccc2sc(c(C)c2c1)-c3ccnc(N)n3)c4ccccc4 |
| 438 | Cc1c(sc2ccc(cc12)C(═O)NCC3(O)CCCCC3)-c4ccnc(N)n4 |
| 439 | COc1cc(CNC(═O)c2ccc3sc(c(C)c3c2)-c4ccnc(N)n4)cc(OC)c1 |
| 440 | COc1ccc(CNC(═O)c2ccc3sc(c(C)c3c2)-c4ccnc(N)n4)cc1OC |
| 441 | Cc1c(sc2ccc(cc12)C(═O)NCC(═O)c3ccccc3)-c4ccnc(N)n4 |
| 442 | CCOC(═O)N1CCC(CC1)NC(═O)c2ccc3sc(c(C)c3c2)-c4ccnc(N)n4 |
| 443 | Cc1c(sc2ccc(cc12)C(═O)NCCCNC(═O)OC(C)(C)C)-c3ccnc(N)n3 |
| 444 | Cc1c(sc2ccc(cc12)C(═O)NCc3ccc(cc3)C(F)(F)F)-c4ccnc(N)n4 |
| 445 | Cc1c(sc2ccc(cc12)C(═O)NCc3cccc(c3)C(F)(F)F)-c4ccnc(N)n4 |
| 446 | Cc1c(sc2ccc(cc12)C(═O)NCc3ccc(Cl)c(Cl)c3)-c4ccnc(N)n4 |
| 447 | Cc1c(sc2ccc(cc12)C(═O)NCc3ccc(Cl)cc3Cl)-c4ccnc(N)n4 |
| 448 | Cc1c(sc2ccc(cc12)C(═O)NC3CCN(C3)Cc4ccccc4)-c5ccnc(N)n5 |
| 449 | COc1ccc(OC)c(CCNC(═O)c2ccc3sc(c(C)c3c2)-c4ccnc(N)n4)c1 |
| 450 | CN(C)c1ccc(CNC(═O)c2ccc3sc(c(C)c3c2)-c4ccnc(N)n4)cc1 |
| 451 | COc1ccc(CNC(═O)c2ccc3sc(c(C)c3c2)-c4ccnc(N)n4)cc1O |
| 452 | Cc1c(sc2ccc(cc12)C(═O)NC3CCN(CC3)Cc4ccccc4)-c5ccnc(N)n5 |
| 453 | Cc1c(sc2ccc(cc12)C(═O)NCc3ccccc3OC(F)(F)F)-c4ccnc(N)n4 |
| 454 | Cc1c(sc2ccc(cc12)C(═O)NCCc3ccc(cc3)S(N)(═O)═O)-c4ccnc(N)n4 |
| 455 | COc1ccc(cc1)C(═O)CNC(═O)c2ccc3sc(c(C)c3c2)-c4ccnc(N)n4 |
| 456 | Cc1c(sc2ccc(cc12)C(═O)NCCc3ccc4OCOc4c3)-c5ccnc(N)n5 |
| 457 | Cc1c(sc2ccc(cc12)C(═O)NCCC(c3ccccc3)c4ccccc4)-c5ccnc(N)n5 |
| 458 | Cc1ccc(cc1)S(═O)(═O)NCCNC(═O)c2ccc3sc(c(C)c3c2)-c4ccnc(N)n4 |
| 459 | CN(C)C(═O)c1ccc2sc(c(C)c2c1)-c3ccnc(N)n3 |
| 460 | CCN(C)C(═O)c1ccc2sc(c(C)c2c1)-c3ccnc(N)n3 |
| 461 | CN(CC#C)C(═O)c1ccc2sc(c(C)c2c1)-c3ccnc(N)n3 |
| 462 | CCN(CC)C(═O)c1ccc2sc(c(C)c2c1)-c3ccnc(N)n3 |
| 463 | CCCN(C)C(═O)c1ccc2sc(c(C)c2c1)-c3ccnc(N)n3 |
| 464 | CN(CCO)C(═O)c1ccc2sc(c(C)c2c1)-c3ccnc(N)n3 |
| 465 | CN(CCC#N)C(═O)c1ccc2sc(c(C)c2c1)-c3ccnc(N)n3 |
| 466 | Cc1c(sc2ccc(cc12)C(═O)N3CCOCC3)-c4ccnc(N)n4 |
| 467 | CC(C)CN(C)C(═O)c1ccc2sc(c(C)c2c1)-c3ccnc(N)n3 |
| 468 | CCN(C(C)C)C(═O)c1ccc2sc(c(C)c2c1)-c3ccnc(N)n3 |
| 469 | CCCCN(C)C(═O)c1ccc2sc(c(C)c2c1)-c3ccnc(N)n3 |
| 470 | CCN(CCO)C(═O)c1ccc2sc(c(C)c2c1)-c3ccnc(N)n3 |
| 471 | Cc1c(sc2ccc(cc12)C(═O)N3CCSC3)-c4ccnc(N)n4 |
| 472 | CC1CCCCN1C(═O)c2ccc3sc(c(C)c3c2)-c4ccnc(N)n4 |
| 473 | CC1CCN(CC1)C(═O)c2ccc3sc(c(C)c3c2)-c4ccnc(N)n4 |
| 474 | CN1CCN(CC1)C(═O)c2ccc3sc(c(C)c3c2)-c4ccnc(N)n4 |
| 475 | Cc1c(sc2ccc(cc12)C(═O)N3CCCC3CO)-c4ccnc(N)n4 |
| 476 | Cc1c(sc2ccc(cc12)C(═O)N3CCC[C@@H]3CO)-c4ccnc(N)n4 |
| 477 | CCCCN(CC)C(═O)c1ccc2sc(c(C)c2c1)-c3ccnc(N)n3 |
| 478 | CCCN(CCC)C(═O)c1ccc2sc(c(C)c2c1)-c3ccnc(N)n3 |
| 479 | Cc1c(sc2ccc(cc12)C(═O)N3CCSCC3)-c4ccnc(N)n4 |
| 480 | Cc1c(sc2ccc(cc12)C(═O)N(CCO)CCO)-c3ccnc(N)n3 |
| 481 | CC1CC(C)CN(C1)C(═O)c2ccc3sc(c(C)c3c2)-c4ccnc(N)n4 |
| 482 | CN(C1CCCCC1)C(═O)c2ccc3sc(c(C)c3c2)-c4ccnc(N)n4 |
| 483 | CC1CCCC(C)N1C(═O)c2ccc3sc(c(C)c3c2)-c4ccnc(N)n4 |
| 484 | CN(C)C1CCN(C1)C(═O)c2ccc3sc(c(C)c3c2)-c4ccnc(N)n4 |
| 485 | C[C@@H]1CN(C[C@H](C)N1)C(═O)c2ccc3sc(c(C)c3c2)-c4ccnc(N)n4 |
| 486 | Cc1c(sc2ccc(cc12)C(═O)N3CCCC(CO)C3)-c4ccnc(N)n4 |
| 487 | COC[C@@H]1CCCN1C(═O)c2ccc3sc(c(C)c3c2)-c4ccnc(N)n4 |
| 488 | CC1CN(CC(C)O1)C(═O)c2ccc3sc(c(C)c3c2)-c4ccnc(N)n4 |
| 489 | Cc1c(sc2ccc(cc12)C(═O)N3CCCCC3CO)-c4ccnc(N)n4 |
| 490 | CCN(CCCCO)C(═O)c1ccc2sc(c(C)c2c1)-c3ccnc(N)n3 |
| 491 | Cc1c(sc2ccc(cc12)C(═O)N3Cc4ccccc4C3)-c5ccnc(N)n5 |
| 492 | CN(Cc1ccccc1)C(═O)c2ccc3sc(c(C)c3c2)-c4ccnc(N)n4 |
| 493 | CCN(C1CCCCC1)C(═O)c2ccc3sc(c(C)c3c2)-c4ccnc(N)n4 |
| 494 | Cc1c(sc2ccc(cc12)C(═O)N3CCC(CC3)C(N)═O)-c4ccnc(N)n4 |
| 495 | CC(═O)N[C@H]1CCN(C1)C(═O)c2ccc3sc(c(C)c3c2)-c4ccnc(N)n4 |
| 496 | Cc1c(sc2ccc(cc12)C(═O)N3CCCC(C3)C(N)═O)-c4ccnc(N)n4 |
| 497 | Cc1c(sc2ccc(cc12)C(═O)N3CCCCC3CCO)-c4ccnc(N)n4 |
| 498 | Cc1c(sc2ccc(cc12)C(═O)N3CCc4ccccc4C3)-c5ccnc(N)n5 |
| 499 | CCN(Cc1ccccc1)C(═O)c2ccc3sc(c(C)c3c2)-c4ccnc(N)n4 |
| 500 | CN(CCc1ccccc1)C(═O)c2ccc3sc(c(C)c3c2)-c4ccnc(N)n4 |
| 501 | CN(CCc1ccccn1)C(═O)c2ccc3sc(c(C)c3c2)-c4ccnc(N)n4 |
| 502 | Cc1c(sc2ccc(cc12)C(═O)N3CCCC4CCCCC34)-c5ccnc(N)n5 |
| 503 | Cc1c(sc2ccc(cc12)C(═O)N(CC═C)C3CCCCC3)-c4ccnc(N)n4 |
| 504 | COC(═O)C1CCN(CC1)C(═O)c2ccc3sc(c(C)c3c2)-c4ccnc(N)n4 |
| 505 | CC(C)N(Cc1ccccc1)C(═O)c2ccc3sc(c(C)c3c2)-c4ccnc(N)n4 |
| 506 | Cc1c(sc2ccc(cc12)C(═O)N(CCO)Cc3ccccc3)-c4ccnc(N)n4 |
| 507 | CN(CC(O)c1ccccc1)C(═O)c2ccc3sc(c(C)c3c2)-c4ccnc(N)n4 |
| 508 | Cc1c(sc2ccc(cc12)C(═O)N3CCC(O)(O)CC3)-c4ccnc(N)n4 |
| 509 | CCOC(═O)C1CCN(CC1)C(═O)c2ccc3sc(c(C)c3c2)-c4ccnc(N)n4 |
| 510 | CCOC(═O)C1CCCN(C1)C(═O)c2ccc3sc(c(C)c3c2)-c4ccnc(N)n4 |
| 511 | CCOC(═O)N1CCN(CC1)C(═O)c2ccc3sc(c(C)c3c2)-c4ccnc(N)n4 |
| 512 | Cc1c(sc2ccc(cc12)C(═O)N(CCC#N)Cc3cccnc3)-c4ccnc(N)n4 |
| 513 | Cc1c(sc2ccc(cc12)C(═O)N3CCN(CC3)c4ccccc4)-c5ccnc(N)n5 |
| 514 | Cc1c(sc2ccc(cc12)C(═O)N3CCN(CC3)c4ccccn4)-c5ccnc(N)n5 |
| 515 | CCCCN(Cc1ccccc1)C(═O)c2ccc3sc(c(C)c3c2)-c4ccnc(N)n4 |
| 516 | C[C@@H](N(CCO)C(═O)c1ccc2sc(c(C)c2c1)-c3ccnc(N)n3)c4ccccc4 |
| 517 | Cc1c(sc2ccc(cc12)C(═O)N(CCCO)Cc3ccccn3)-c4ccnc(N)n4 |
| 518 | CN(CC(O)c1ccc(O)cc1)C(═O)c2ccc3sc(c(C)c3c2)-c4ccnc(N)n4 |
| 519 | Cc1c(sc2ccc(cc12)C(═O)N3CCN(CC3)C4CCCCC4)-c5ccnc(N)n5 |
| 520 | Cc1c(sc2ccc(cc12)C(═O)N3CCC(CC3)Cc4ccccc4)-c5ccnc(N)n5 |
| 521 | Cc1c(sc2ccc(cc12)C(═O)N3CCN(CC3)Cc4ccccc4)-c5ccnc(N)n5 |
| 522 | Cc1c(sc2ccc(cc12)C(═O)N3CCCN(CC3)Cc4ccccc4)-c5ccnc(N)n5 |
| 523 | Cc1c(sc2ccc(cc12)C(═O)N3CCN(CC3)c4ccc(O)cc4)-c5ccnc(N)n5 |
| 524 | CN(C)CCN(Cc1ccccc1)C(═O)c2ccc3sc(c(C)c3c2)-c4ccnc(N)n4 |
| 525 | Cc1c(sc2ccc(cc12)C(═O)N3CCN(CC3)c4ccccc4F)-c5ccnc(N)n5 |
| 526 | Cc1c(sc2ccc(cc12)C(═O)N3CCN(CC3)c4ccc(F)cc4)-c5ccnc(N)n5 |
| 527 | Cc1c(sc2ccc(cc12)C(═O)N3CCN(CC3)CC4CCCCC4)-c5ccnc(N)n5 |
| 528 | CN(C[C@H](O)c1ccc(O)c(O)c1)C(═O)c2ccc3sc(c(C)c3c2)-c4ccnc(N)n4 |
| 529 | CCOC(═O)CC1N(CCNC1═O)C(═O)c2ccc3sc(c(C)c3c2)-c4ccnc(N)n4 |
| 530 | Cc1c(sc2ccc(cc12)C(═O)N3CCN(CC3)C(═O)OC(C)(C)C)-c4ccnc(N)n4 |
| 531 | Cc1c(sc2ccc(cc12)C(═O)N3CCC(C3)NC(═O)OC(C)(C)C)-c4ccnc(N)n4 |
| 532 | Cc1c(sc2ccc(cc12)C(═O)N3CCN(CC3)c4ccccc4C#N)-c5ccnc(N)n5 |
| 533 | Cc1c(sc2ccc(cc12)C(═O)N3CCN(CC3)c4ccc(cn4)C#N)-c5ccnc(N)n5 |
| 534 | Cc1cccc(N2CCN(CC2)C(═O)c3ccc4sc(c(C)c4c3)-c5ccnc(N)n5)c1C |
| 535 | CC1CN(CCN1c2cccc(C)c2)C(═O)c3ccc4sc(c(C)c4c3)-c5ccnc(N)n5 |
| 536 | Cc1c(sc2ccc(cc12)C(═O)N3CCN(CC3)CCc4ccccc4)-c5ccnc(N)n5 |
| 537 | Cc1ccc(cc1C)N2CCN(CC2)C(═O)c3ccc4sc(c(C)c4c3)-c5ccnc(N)n5 |
| 538 | Cc1ccc(N2CCN(CC2)C(═O)c3ccc4sc(c(C)c4c3)-c5ccnc(N)n5)c(C)c1 |
| 539 | Cc1ccc(C)c(c1)N2CCN(CC2)C(═O)c3ccc4sc(c(C)c4c3)-c5ccnc(N)n5 |
| 540 | COc1ccc(cc1)N2CCN(CC2)C(═O)c3ccc4sc(c(C)c4c3)-c5ccnc(N)n5 |
| 541 | COc1ccccc1N2CCN(CC2)C(═O)c3ccc4sc(c(C)c4c3)-c5ccnc(N)n5 |
| 542 | COc1cccc(c1)N2CCN(CC2)C(═O)c3ccc4sc(c(C)c4c3)-c5ccnc(N)n5 |
| 543 | COc1ccc(CCN(C)C(═O)c2ccc3sc(c(C)c3c2)-c4ccnc(N)n4)cc1OC |
| 544 | Cc1c(sc2ccc(cc12)C(═O)N3CCN(CC3)c4cccc(Cl)c4)-c5ccnc(N)n5 |
| 545 | Cc1c(sc2ccc(cc12)C(═O)N3CCN(CC3)c4ccc(Cl)cc4)-c5ccnc(N)n5 |
| 546 | Cc1c(sc2ccc(cc12)C(═O)N3CCN(CC3)c4ccc(F)cc4F)-c5ccnc(N)n5 |
| 547 | Cc1c(sc2ccc(cc12)C(═O)N(Cc3cccnc3)Cc4cccnc4)-c5ccnc(N)n5 |
| 548 | Cc1c(sc2ccc(cc12)C(═O)N3CCN(CCN4CCOCC4)CC3)-c5ccnc(N)n5 |
| 549 | CN(C1CCN(C1)C(═O)c2ccc3sc(c(C)c3c2)-c4ccnc(N)n4)C(═O)OC(C)(C)C |
| 550 | CC(═O)c1ccc(cc1)N2CCN(CC2)C(═O)c3ccc4sc(c(C)c4c3)-c5ccnc(N)n5 |
| 551 | CCN(CC)CCN(Cc1ccccc1)C(═O)c2ccc3sc(c(C)c3c2)-c4ccnc(N)n4 |
| 552 | Cc1c(sc2ccc(cc12)C(═O)N3CCN(CC3)c4ccc(cc4)[N+]([O−])═O)-c5ccnc(N)n5 |
| 553 | CN(Cc1cccc2ccccc12)C(═O)c3ccc4sc(c(C)c4c3)-c5ccnc(N)n5 |
| 554 | Cc1ccc(Cl)cc1N2CCN(CC2)C(═O)c3ccc4sc(c(C)c4c3)-c5ccnc(N)n5 |
| 555 | COc1cc(CN(C)C(═O)c2ccc3sc(c(C)c3c2)-c4ccnc(N)n4)cc(OC)c1OC |
| 556 | Cc1c(sc2ccc(cc12)C(═O)N(CCc3ccccc3)Cc4ccccc4)-c5ccnc(N)n5 |
| 557 | CN(C(Cc1ccccc1)c2ccccc2)C(═O)c3ccc4sc(c(C)c4c3)-c5ccnc(N)n5 |
| 558 | Cc1c(sc2ccc(cc12)C(═O)N3CCC(O)(CC3)c4ccc(Cl)cc4)-c5ccnc(N)n5 |
| 559 | Cc1c(sc2ccc(cc12)C(═O)N(CC#C)Cc3ccc(Cl)cc3Cl)-c4ccnc(N)n4 |
| 560 | CCN(C(Cc1ccccc1)c2ccco2)C(═O)c3ccc4sc(c(C)c4c3)-c5ccnc(N)n5 |
| 561 | Cc1c(sc2ccc(cc12)C(═O)N3CCN(C[C@H]3CO)C(═O)OC(C)(C)C)- |
| | c4ccnc(N)n4 |
| 562 | Cc1c(sc2ccc(cc12)C(═O)N3CCN(CC3)c4ccc(cc4)C(C)(C)C)-c5ccnc(N)n5 |
| 563 | Cc1c(sc2ccc(cc12)C(═O)N3CCN(CC3)Cc4ccc5OCOc5c4)-c6ccnc(N)n6 |
| 564 | COc1cc2CCN(Cc2cc1OC)C(═O)c3ccc4sc(c(C)c4c3)-c5ccnc(N)n5 |
| 565 | Cc1c(sc2ccc(cc12)C(═O)N3CCN(CC3)c4ccc(cc4)C(F)(F)F)-c5ccnc(N)n5 |
| 566 | Cc1c(sc2ccc(cc12)C(═O)N3CCN(CC3)c4cccc(c4)C(F)(F)F)-c5ccnc(N)n5 |
| 567 | Cc1c(sc2ccc(cc12)C(═O)N3CCN(CC3)c4ccc(Cl)c(Cl)c4)-c5ccnc(N)n5 |
| 568 | Cc1c(sc2ccc(cc12)C(═O)N3CCN(CC3)c4ccc(cn4)C(F)(F)F)-c5ccnc(N)n5 |
| 569 | Cc1c(sc2ccc(cc12)C(═O)N3CCN(CC3)c4ccccc4Cl)-c5ccnc(N)n5 |
| 570 | COc1ccc(cc1OC)N2CCN(CC2)C(═O)c3ccc4sc(c(C)c4c3)-c5ccnc(N)n5 |
| 571 | Cc1c(sc2ccc(cc12)C(═O)N3CCCC(C3)c4ccc(cc4)C(F)(F)F)-c5ccnc(N)n5 |

The following compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36. All SMILES strings used herein, as well as many IUPAC names, were generated using CambridgeSoft's ChemDraw 10.0.

The following compounds can generally be made using the methods described above. It is expected that these compounds when made will have activity similar to those that have been made in the examples above.

CC1=C(C2=NC(N)═NC═C2)N═C3C═NC═CN31

CC4=C(C5=NON═C5N)N═C6C═NC═CN64

CC1=C(C2=NC(N)═NC═C2)N═C3C═CC═CN31

CC4=C(C5=NON═C5N)N═C6C═CC═CN64

NC1=NC═CC(C2=C(CC)N3C═CN═CC3=N2)=N1

NC4=NON═C4C5=C(CC)N6C═CN═CC6=N5

NC1=NC═CC(C2=C(CC)N3C═CC═CC3=N2)=N1

NC4=NON═C4C5=C(CC)N6C═CC═CC6=N5

CC1=C2C═CN═CN2N═C1C3=NC(N)═NC═C3

CC4=C5C═CN═CN5N═C4C6=NON═C6N

CC1=C2C═CN═CN2C═C1C3=NC(N)═NC═C3

CC4=C5C═CN═CN5C═C4C6=NON═C6N

NC1=NC═CC(C2=CN3C═NC═CC3=C2CC)═N1

NC4=NON═C4C5=CN6C═NC═CC6=C5CC

CC1=C2C═CC═CN2N═C1C3=NC(N)═NC═C3

CC4=C5C═CC═CN5N═C4C6=NON═C6N

NC1=NC═CC(C2=NN3C═CC═CC3=C2CC)═N1

NC4=NON═C4C5=NN6C═CC═CC6=C5CC

CC1=C(C2=NC(N)═NC═C2)N═C3C═NC(OC)═CN31

CC4=C(C5=NON═C5N)N═C6C═NC(OC)═CN64

CC1=C(C2=NC(N)═NC═C2)N═C3C═CC(OC)═CN31

CC4=C(C5=NON═C5N)N═C6C═CC(OC)═CN64

CC1=C2C═C(OC)N═CN2N═C1C3=NC(N)═NC═C3

CC4=C5C═C(OC)N═CN5N═C4C6=NON═C6N

CC1=C2C═C(OC)C═CN2N═C1C3=NC(N)═NC═C3

CC4=C5C═C(OC)C═CN5N═C4C6=NON═C6N

CC1=C(C2=NC(N)═NC═C2)N═C3C═NC(NC)═CN31

CC4=C(C5=NON═C5N)N═C6C═NC(NC)═CN64

CC1=C2C═C(NC)N═CN2N═C1C3=NC(N)═NC═C3

CC4=C5C═C(NC)N═CN5N═C4C6=NON═C6N

CC1=C(C2=CC═NC(N)═N2)OC3=CN═CC═C31

CC4=C(C5=NON═C5N)OC6=CN═CC═C64

NC1=NC(C2=C(CC)C3=CC═NC═C3O2)=CC═N1

NC4=NON═C4C5=C(CC)C6=CC═NC═C6O5

CC1=C(C2=CC═NC(N)═N2)SC3=CN═CN═C31

CC4=C(C5=NON═C5N)SC6=CN═CN═C64

CC1=C(C2=CC═NC(N)═N2)SC3=CC═CN═C31

CC4=C(C5=NON═C5N)SC6=CC═CN═C64

CC1=C(C2=CC═NC(N)═N2)SC3=CC═NC═C31

CC4=C(C5=NON═C5N)SC6=CC═NC═C64

CC1=C(C2=CC═NC(N)═N2)SC3=NC═CC═C31

CC4=C(C5=NON═C5N)SC6=NC═CC═C64

NC1=NC(C2=C(CC)C3=NC═NC═C3S2)=CC═N1

NC4=NON═C4C5=C(CC)C6=NC═NC═C6S5

NC1=NC(C2=C(CC)C3=CN═CN═C3S2)=CC═N1

NC4=NON═C4C5=C(CC)C6=CN═CN═C6S5

CC1=C(C2=NC(N)═NC═C2)N═C3C═NC(CC4=CC═CC═C4)=CN31

CC5=C(C6=NON═C6N)N═C7C═NC(CC8⋆CC═CC═C8)=CN75

CC1=C(C2=NC(N)═NC═C2)N═C3C═NC(OC4=CC═CC═C4)=CN31

CC5=C(C6=NON═C6N)N═C7C═NC(OC8═CC═CC═C8)=CN75

CC1=C(C2=NC(N)═NC═C2)N═C3C═NC(SC4=CC═CC═C4)=CN31

CC5=C(C6=NON═C6N)N═C7C═NC(SC8═CC═CC═C8)=CN75

CC1=C(C2=NC(N)═NC═C2)N═C3C═NC(S(C4=CC═CC═C4)(═O)═O)═CN31

CC5=C(C6=NON═C6N)N═C7C═NC(S(C8=CC═CC═C8)(═O)═O)═CN75

CC1=C(C2=NC(N)═NC═C2)N═C3C═NC(NC4=CC═CC═C4)=CN31

CC5=C(C6=NON═C6N)N═C7C═NC(NC8=CC═CC═C8)=CN75

CC1=C2C═C(CC3=CC═CC═C3)N═CN2N═C1C4=NC(N)═NC═C4

CC5=C6C═C(CC7=CC═CC═C7)N═CN6N═C5C8=NON═C8N

CC1=C2C═C(OC3=CC═CC═C3)N═CN2N=C1C4=NC(N)═NC═C4

CC5=C6C═C(OC7=CC═CC═C7)N═CN6N═C5C8=NON═C8N

CC1=C2C═C(SC3=CC═CC═C3)N═CN2N═C1C4=NC(N)═NC═C4

CC5=C6C═C(SC7=CC═CC═C7)N═CN6N═C5C8=NON═C8N

CC1=C2C═C(S(C3=CC═CC═C3)(═O)═O)N═CN2N═C1C4=NC(N)═NC═C4

CC5=C6C═C(S(C7=CC═CC═C7)(═O)═O)N═CN6N═C5C8=NON═C8N

CC1=C2C═C(NC3=CC═CC═C3)N═CN2N═C1C4=NC(N)═NC═C4

CC5=C6C═C(NC7=CC═CC═C7)N═CN6N═C5C8=NON═C8N

CC1=C(C2=NC(N)═NC═C2)N═C3C=NC(CC4=CC═CC(O)═C4)=CN31.CC5=C(C6=NON═C6N)N═C7C═NC(CC8=CC═CC(O)═C8)=CN75

CC1=C(C2=NC(N)═NC═C2)N═C3C═NC(OC4=CC═CC(O)═C4)=CN31

CC5=C(C6=NON═C6N)N═C7C═NC(OC8=CC═CC(O)═C8)=CN75

CC1=C(C2=NC(N)═NC═C2)N═C3C═NC(SC4=CC═CC(O)═C4)=CN31

CC5=C(C6=NON═C6N)N═C7C═NC(SC8=CC═CC(O)═C8)=CN75

CC1=C(C2=NC(N)═NC═C2)N═C3C═NC(S(C4=CC═CC(O)═C4)(=O)═O)═CN31

CC5=C(C6=NON═C6N)N═C7C═NC(S(C8=CC═CC(O)═C8)(=O)═O)═CN75

CC1=C(C2=NC(N)═NC═C2)N═C3C═NC(NC4=CC═CC(O)═C4)=CN31

CC5=C(C6=NON═C6N)N═C7C═NC(NC8=CC═CC(O)═C8)=CN75

CC1=C(C2=NC(N)═NC═C2)N═C3C═NC(CC4=CC═CC(C(O)═O)═C4)=CN31

CC5=C(C6=NON═C6N)N═C7C═NC(CC8=CC═CC(C(O)═O)═C8)=CN75

CC1=C(C2=NC(N)═NC═C2)N═C3C═NC(OC4=CC═CC(C(O)═O)═C4)=CN31

CC5=C(C6=NON═C6N)N═C7C═NC(OC8=CC═CC(C(O)═O)═C8)=CN75

CC1=C(C2=NC(N)═NC═C2)N═C3C═NC(SC4=CC═CC(C(O)═O)═C4)=CN31

CC5=C(C6=NON═C6N)N═C7C═NC(SC8=CC═CC(C(O)═O)═C8)=CN75

CC1=C(C2=NC(N)═NC═C2)N═C3C═NC(NC4=CC═CC(C(O)═O)═C4)=CN31

CC5=C(C6=NON═C6N)N═C7C═NC(NC8=CC═CC(C(O)═O)═C8)=CN75

CC1=C(C2=NC(N)═NC═C2)N═C3C═NC(CC4=CC═CC(C(NC)═O)═C4)=CN31

CC5=C(C6=NON═C6N)N═C7C═NC(CC8=CC═CC(C(NC)═O)═C8)=CN75

CC1=C(C2=NC(N)═NC═C2)N═C3C═NC(OC4=CC═CC(C(NC)═O)═C4)=CN31

CC5=C(C6=NON═C6N)N═C7C═NC(OC8=CC═CC(C(NC)═O)═C8)=CN75

CC1=C(C2=NC(N)═NC═C2)N═C3C═NC(SC4=CC═CC(C(NC)═O)═C4)=CN31

CC5=C(C6=NON═C6N)N═C7C═NC(SC8=CC═CC(C(NC)═O)═C8)=CN75

CC1=C(C2=NC(N)═NC═C2)N═C3C═NC(NC4=CC═CC(C(NC)═O)═C4)=CN31

CC5=C(C6=NON═C6N)N═C7C═NC(NC8=CC═CC(C(NC)═O)═C8)=CN75

CC1=C2C═C(CC3=CC═CC(O)═C3)N═CN2N═C1C4=NC(N)═NC═C4

CC5=C6C═C(CC7=CC═CC(O)═C7)N═CN6N═C5C8=NON═C8N

CC1=C2C═C(OC3=CC═CC(O)═C3)N═CN2N═C1C4=NC(N)═NC═C4

CC5=C6C═C(OC7=CC═CC(O)═C7)N═CN6N═C5C8=NON═C8N

CC1=C2C═C(SC3=CC═CC(O)═C3)N═CN2N═C1C4=NC(N)═NC═C4

CC5=C6C═C(SC7=CC═CC(O)═C7)N═CN6N═C5C8=NON═C8N

CC1=C2C═C(NC3=CC═CC(O)═C3)N═CN2N═C1C4=NC(N)═NC═C4

CC5=C6C═C(NC7=CC═CC(O)═C7)N═CN6N═C5C8=NON═C8N

CC1=C(C2=NC(N)═NC═C2)N═C3C═NC(CC4=CN═CC(O)═C4)=CN31

CC5=C(C6=NON═C6N)N═C7C═NC(CC8=CN═CC(O)═C8)=CN75

CC1=C(C2=NC(N)═NC═C2)N═C3C═NC(OC4=CN═CC(O)═C4)=CN31

CC5=C(C6=NON═C6N)N═C7C═NC(OC8=CN═CC(O)═C8)=CN75

CC1=C(C2=NC(N)═NC═C2)N═C3C═NC(SC4=CN═CC(O)═C4)=CN31

CC5=C(C6=NON═C6N)N═C7C═NC(SC8=CN═CC(O)═C8)=CN75

CC1=C(C2=NC(N)═NC═C2)N═C3C═NC(S(C4=CN═CC(O)═C4)(=O)═O)═CN31

CC5=C(C6=NON═C6N)N═C7C═NC(S(C8=CN═CC(O)═C8)(=O)═O)═CN75

CC1=C(C2=NC(N)═NC═C2)N═C3C═NC(NC4=CN═CC(O)═C4)=CN31

CC5=C(C6=NON═C6N)N═C7C═NC(NC8=CN═CC(O)═C8)=CN75

CC1=C(C2=NC(N)═NC═C2)N═C3C═NC(NC4=CN═CC═C4)=CN31

CC5=C(C6=NON═C6N)N═C7C═NC(NC8=CN═CC═C8)=CN75

CC1=C(C2=CC═NC(N)═N2)SC3=CN═C(CC4=CC═CC(C(O)═O)═C4)C═C31

CC5=C(C6=CC═NC(N)═N6)SC7=CN═C(CC8=CC═CC(OC)═C8)C═C75

CC1=C(C2=CC═NC(N)═N2)SC3=CC═C(CC4=CN═CC(C(O)═O)═C4)C═C31

CC5=C(C6=CC═NC(N)═N6)SC7=CC═C(CC8=CN═CC(OC)═C8)C═C75

CC1=C(C2=CC═NC(N)═N2)SC3=CN═C(CC4=CN═CC(C(O)═O)═C4)C═C31

CC5=C(C6=CC═NC(N)═N6)SC7=CN═C(CC8=CN═CC(OC)═C8)C═C75

CC1=C(C2=CC═NC(N)═N2)SC3=CN═C(NC4=CC═CC(C(O)═O)═C4)C═C31

CC5=C(C6=CC═NC(N)═N6)SC7=CN═C(NC8=CC═CC(OC)═C8)C═C75

CC1=C(C2=CC═NC(N)═N2)SC3=CC═C(CC4=CN═CC(O)═C4)C═C31

CC5=C(C6=CC═NC(N)═N6)SC7=CN═C(CC8=CC═CC(O)═C8)C═C75

CC1=C(C2=CC═NC(N)═N2)SC3=CN═C(CC4=CN═CC(O)═C4)C═C31

CC5=C(C6=CC═NC(N)═N6)SC7=CN═C(NC8=CC═CC(O)═C8)C═C75

CC1=C(C2=CC═NC(N)═N2)SC3=CC═C(NC4=CNC═N4)C═C31

CC5=C(C6=CC═NC(N)═N6)SC7=CC═C(CC8=CNN═C8)C═C75

NC1=NC═CC(C2=C(CC)N3C═C(NC4=CNN═C4)N═CC3=N2)=N1

NC5=NON═C5C6=C(CC)N7C═C(NC8=CNC═N8)N═CC7=N6

CC1=C(C2=NC(N)═NC═C2)N═C3C═NC(NCCCO)═CN31

CC4=C(C5=NON═C5N)N═C6C═NC(NCCCO)═CN64

CC1=C2C═C(NCCCO)N═CN2N═C1C3=NC(N)═NC═C3

CC4=C5C═C(NCCCO)N═CN5N═C4C6=NON═C6N

CC1=C(C2=NC(N)═NC═C2)N═C3C═NC(OCCCO)═CN31

CC4=C(C5=NON═C5N)N═C6C═NC(OCCCO)═CN64

NC1=NC═CC(C2=C(CC)N3C═C(OCCO)N═CC3=N2)=N1

NC4=NON═C4C5=C(CC)N6C═C(OCCO)N═CC6=N5

NC1=NC═CC(C2=C(CC)N3C═C(NCCO)N═CC3=N2)=N1

NC4=NON═C4C5=C(CC)N6C═C(NCCO)N═CC6=N5

CC1=C2C═C(NCCO)N═CN2N═C1C3=NC(N)═NC═C3

CC4=C5C═C(NCCO)N═CN5N═C4C6=NON═C6N

CC1=C2C═C(OCCCO)N═CN2N═C1C3=NC(N)═NC═C3

CC4=C5C═C(OCCCO)N═CN5N═C4C6=NON═C6N

CC1=C2C═C(OCCO)N═CN2N═C1C3=NC(N)═NC═C3

CC4=C5C═C(OCCO)N═CN5N═C4C6=NON═C6N

CC1=C(C2=NC(N)═NC═C2)N═C3C═NC(NCC(N)CO)═CN31

CC4=C(C5=NON═C5N)N═C6C═NC(NCC(N)CO)═CN64

CC1=C2C═C(NCC(N)CO)N═CN2N═C1C3=NC(N)═NC═C3

CC4=C5C═C(NCC(N)CO)N═CN5N═C4C6=NON═C6N

CC1=C(C2=CC═NC(N)═N2)OC3=CN═C(OC4=CC═CC(O)═C4)C═C31

CC5=C(C6=NON═C6N)OC7=CN═C(OC8=CC═CC(O)═C8)C═C75

CC1=C(C2=CC═NC(N)═N2)OC3=CN═C(OC4=CN═CC(O)═C4)C═C31

CC5=C(C6=NON═C6N)OC7=CN═C(OC8=CN═CC(O)═C8)C═C75

CC1=C(C2=CC═NC(N)═N2)OC3=CN═C(NCCO)C═C31

CC4=C(C5=NON═C5N)OC6=CN═C(NCCO)C═C64

CC1=C(C2=CC═NC(N)═N2)OC3=CN═C(NCCCO)C═C31

CC4=C(C5=NON═C5N)OC6=CN═C(NCCCO)C═C64

NC1=NC(C2=C(CC)C3=NC(OC4=CC═CC(O)═C4)=NC═C3S2)=CC═N1

NC5=NON═C5C6=C(CC)C7=NC(OC8=CC═CC(O)═C8)=NC═C7S6

NC1=NC(C2=C(CC)C3=NC(OCCCO)═NC═C3S2)=CC═N1

NC4=NON═C4C5=C(CC)C6=NC(OCCCO)═NC═C6S5

NC1=NC(C2=C(CC)C3=CC(C(N)C4=CC═CC(OC)═C4)=CC═C3S2)=CC═N1

NC5=NON═C5C6=C(CC)C7=CC(C(N)C8=CC═CC(OC)═C8)=CC═C7S6

NC1=NC(C2=C(CC)C3=CC(C(N)C4=CC═CC(O)═C4)=CC═C3S2)=CC═N1

NC5=NON═C5C6=C(CC)C7=CC(C(N)C8=CC═CC(O)═C8)=CC═C7S6

NC1=NC(C2=C(CC)C3=CC(C(N)C4=CC═CC(OC)═C4)=NC═C3S2)=CC═N1

NC5=NON═C5C6=C(CC)C7=CC(C(N)C8=CC═CC(OC)═C8)=NC═C7S6

NC1=NC(C2=C(CC)C3=CC(C(N)C4=CC═CC(O)═C4)=NC═C3S2)=CC═N1

NC5=NON═C5C6=C(CC)C7=CC(C(N)C8=CC═CC(O)═C8)=NC═C7S6

CC1=C(C2=CC═NC(N)═N2)SC3=CN═C(C(N)C4=CC═CC(O)═C4)C═C31

CC5=C(C6=NON═C6N)SC7=CN═C(C(N)C8=CC═CC(O)═C8)C═C75

NC1=NC(C2=C(CC)C3=CC(C(N)C4=CC═CC(O)═C4)=CC═C3O2)=CC═N1

NC5=NON═C5C6=C(CC)C7=CC(C(N)C8=CC═CC(O)═C8)=CC═C7O6

NC1=NC(C2=C(CC)C3=CC(C(N(C)C)C4=CC═CC(O)═C4)=CC═C3S2)=CC═N1

NC5=NON═C5C6=C(CC)C7=CC(C(N(C)C)C8=CC═CC(O)═C8)=CC═C7S6

CC1=C(C2=CC═NC(N)═N2)SC3=CN═C(C(N4CCOCC4)C5=CC═CC(O)═C5)C═C31

CC6=C(C7=NON═C7N)SC8=CN═C(C(N9CCOCC9)C % 10=CC═CC(O)═C % 10)C═C 86

CC1=C(C2=CC═NC(N)═N2)SC3=CC═C(C(N4CCNCC4)C5=CC═CC(O)═C5)C═C31

CC6=C(C7=NON═C7N)SC8=CC═C(C(N9CCNCC9)C % 10=CC═CC(O)═C % 10)C═C 86

The activity of the compounds in Examples 1-570 as Rho kinase inhibitor is illustrated in the following assay. The other compounds listed above, which have not yet been made or tested, are predicted to have activity in this assay as well.

### Biological Activity Assay

In Vitro Rho Kinase Assay

Rho kinase biochemical assays described below depend on firefly luciferase-based, indirect measurement of total ATP consumption by the kinase following incubation with substrate and ATP. 25 μl of Rho kinase assay buffer (20 mM Tris-HCL [pH 7.5], 1 mM MgCl₂, 0.4 mM CaCl₂, 0.15 mM EGTA, 0.1 mg/ml bovine serum albumin) containing 0.82 μg/ml of recombinant N-terminal GST-tagged human Rho kinase 1 (ROCK1, amino acids 1-535, Invitrogen Inc., cat. #PV-3691) or recombinant N-terminal GST-tagged human Rho kinase 2 (ROCK2, amino acids 1-552, Invitrogen Inc., cat #PV3759), 100 μg/ml S6 peptide substrate (related to amino acids 218-249 of the human 40S ribosomal protein S6, and suitable for ROCK1 or ROCK2, e.g. Upstate/Millipore Inc., cat #12-420), and 3 M ATP are dispensed to wells of a 384 multi-well opaque plate. The plate is centrifuged for 30 seconds at approximately 200×g. 240 nl of test compound in DMSO is dispensed to each well by passive pin transfer. The lag phase of this in vitro kinase reaction permits addition of compounds soon after the reaction initiates. The reaction is allowed to incubate at 30° C. for 2 hours. The assay plates are sealed and maintained in a humidified environment. After 2 hours, 25 μl of easylite protein kinase assay reagent (Perkin-Elmer, Inc.) is dispensed. After an additional 10 minute incubation at room temperature (about 22° C.), luminescence activity is measured on a Molecular Devices Analyst multi-mode plate reader or other suitable plate reader. Kinase inhibition results in less ATP consumption, and therefore increased luminescence signal. Negative control activity is measured with DMSO lacking any test compound. The positive control is 2-methyl-1-(4-methylisoquinolin-5-ylsulfonyl)perhydro-1,4-diazepine hydrochloride (aka H-1152P, HCl salt). Efficacy is measured as a percentage of positive control activity. 50% inhibitory concentration of compound (IC50) is measured by assay in dose response. In some cases, kinase reactions and compound testing are performed in 1536 multi-well plates under similar conditions, with assay volumes appropriately scaled. The designation NT means the cited example was not tested.

TABLE 1
Biological Activity

| | ROCK1 IC₅₀ | ROCK2 IC₅₀ |
|---|---|---|
| | + indicates | + indicates |
| | ≦5 uM | ≦5 uM |
| | − indicates | − indicates |
| Example | >5 uM | >5 uM |
| 1 | + | + |
| 2 | + | + |
| 3 | + | + |
| 4 | − | − |
| 5 | − | − |
| 6 | − | − |
| 7 | − | − |
| 8 | − | − |
| 9 | − | − |
| 10 | + | + |
| 11 | − | − |
| 12 | − | − |
| 13 | + | + |
| 14 | + | + |
| 15 | + | + |
| 16 | + | + |
| 17 | − | − |
| 18 | + | + |
| 19 | + | − |
| 20 | + | + |
| 21 | + | + |
| 22 | − | − |
| 23 | + | + |
| 24 | + | + |
| 25 | − | − |
| 26 | + | + |
| 27 | + | + |
| 28 | + | + |
| 29 | + | + |
| 30 | + | + |
| 31 | + | + |
| 32 | + | + |
| 33 | + | + |
| 34 | + | + |
| 35 | + | + |
| 36 | + | + |
| 37 | − | − |
| 38 | − | − |
| 39 | − | − |
| 40 | + | + |
| 41 | + | + |
| 42 | − | + |
| 43 | + | + |
| 44 | + | + |
| 45 | + | + |
| 46 | + | + |
| 47 | + | + |
| 48 | + | + |
| 49 | + | + |
| 50 | + | + |
| 51 | + | + |
| 52 | + | + |
| 53 | + | + |
| 54 | − | − |
| 55 | + | + |
| 56 | + | + |
| 57 | + | + |
| 58 | + | + |
| 59 | + | + |
| 60 | + | + |
| 61 | + | + |
| 62 | + | + |
| 63 | + | + |
| 64 | + | + |
| 65 | + | + |
| 66 | + | + |
| 67 | + | + |
| 68 | + | + |
| 69 | + | + |
| 70 | + | + |
| 71 | + | + |
| 72 | + | + |
| 73 | − | − |
| 74 | + | + |
| 75 | + | + |
| 76 | + | + |
| 77 | + | + |
| 78 | + | + |
| 79 | + | + |
| 80 | + | + |
| 81 | + | + |
| 82 | + | + |
| 83 | + | + |
| 84 | + | + |
| 85 | + | + |
| 86 | + | + |
| 87 | − | + |
| 88 | + | − |
| 89 | − | − |
| 90 | − | − |
| 91 | + | + |
| 92 | + | + |
| 93 | + | + |
| 94 | + | + |
| 95 | − | − |
| 96 | − | − |
| 97 | + | + |
| 98 | − | + |
| 99 | + | + |
| 100 | + | + |
| 101 | + | + |
| 102 | + | + |
| 103 | + | + |
| 104 | + | + |
| 105 | − | + |
| 106 | − | + |
| 107 | − | + |
| 108 | + | − |
| 109 | − | + |
| 110 | − | + |
| 111 | − | + |
| 112 | − | + |
| 113 | − | + |
| 114 | − | + |
| 115 | − | + |
| 116 | − | + |
| 117 | − | + |
| 118 | − | − |
| 119 | − | + |
| 120 | − | + |
| 121 | + | + |
| 122 | − | + |
| 123 | − | + |
| 124 | − | + |
| 125 | − | + |
| 126 | + | + |
| 127 | + | + |
| 128 | + | + |
| 129 | + | + |
| 130 | + | + |
| 131 | + | + |
| 132 | + | + |
| 133 | + | + |
| 134 | + | + |
| 135 | − | − |
| 136 | − | − |
| 137 | − | − |
| 138 | − | + |
| 139 | − | + |
| 140 | − | + |
| 141 | − | + |
| 142 | − | + |
| 143 | − | + |
| 144 | − | + |
| 145 | − | + |
| 146 | − | + |
| 147 | − | + |
| 148 | − | + |
| 149 | − | + |
| 150 | + | + |
| 151 | + | + |
| 152 | + | + |
| 153 | + | + |
| 154 | − | + |
| 155 | − | + |
| 156 | + | + |
| 157 | − | + |
| 158 | − | − |
| 159 | + | + |
| 160 | + | + |
| 161 | + | + |
| 162 | − | + |
| 163 | + | + |
| 164 | − | + |
| 165 | − | + |
| 166 | − | − |
| 167 | − | + |
| 168 | + | + |
| 169 | + | + |
| 170 | − | + |
| 171 | − | − |
| 172 | + | + |
| 173 | − | + |
| 174 | − | − |
| 175 | − | + |
| 176 | − | + |
| 177 | − | + |
| 178 | + | + |
| 179 | − | + |
| 180 | + | + |
| 181 | + | + |
| 182 | − | + |
| 183 | − | + |
| 184 | − | + |
| 185 | − | + |
| 186 | + | + |
| 187 | + | + |
| 188 | + | + |
| 189 | + | + |
| 190 | + | + |
| 191 | − | + |
| 192 | − | + |
| 193 | + | − |
| 194 | − | + |
| 195 | + | + |
| 196 | + | + |
| 197 | + | − |
| 198 | − | + |
| 199 | − | + |
| 200 | + | + |
| 201 | − | + |
| 202 | + | + |
| 203 | − | − |
| 204 | + | + |
| 205 | + | + |
| 206 | + | + |
| 207 | + | + |
| 208 | + | + |
| 209 | + | + |
| 210 | + | + |
| 211 | + | + |
| 212 | + | + |
| 213 | + | − |
| 214 | − | − |
| 215 | − | + |
| 216 | − | + |
| 217 | − | + |
| 218 | − | + |
| 219 | − | + |
| 220 | − | + |
| 221 | − | + |
| 222 | − | + |
| 223 | − | + |
| 224 | − | + |
| 225 | − | + |
| 226 | − | + |
| 227 | − | + |
| 228 | − | + |
| 229 | + | + |
| 230 | + | + |
| 231 | + | + |
| 232 | − | + |
| 233 | + | + |
| 234 | + | + |
| 235 | + | + |
| 236 | + | + |
| 237 | + | + |
| 238 | + | + |
| 239 | − | + |
| 240 | + | + |
| 241 | + | − |
| 242 | + | − |
| 243 | + | + |
| 244 | + | + |
| 245 | − | + |
| 246 | + | + |
| 247 | + | + |
| 248 | + | + |
| 249 | − | + |
| 250 | − | + |
| 251 | + | − |
| 252 | + | + |
| 253 | + | + |
| 254 | − | + |
| 255 | + | + |
| 256 | + | + |
| 257 | − | − |
| 258 | + | + |
| 259 | + | + |
| 260 | − | + |
| 261 | − | + |
| 262 | − | + |
| 263 | − | + |
| 264 | − | + |
| 265 | − | + |
| 266 | − | + |
| 267 | − | + |
| 268 | − | + |
| 269 | + | + |
| 270 | + | + |
| 271 | − | + |
| 272 | − | + |
| 273 | + | + |
| 274 | − | − |
| 275 | + | − |
| 276 | + | + |
| 277 | + | + |
| 278 | + | + |
| 279 | − | + |
| 280 | − | + |
| 281 | − | + |
| 282 | − | − |
| 283 | + | − |
| 284 | − | + |
| 285 | − | + |
| 286 | − | − |
| 287 | − | + |
| 288 | + | + |
| 289 | + | − |
| 290 | − | − |
| 291 | − | + |
| 292 | + | − |
| 293 | − | − |
| 294 | + | + |
| 295 | + | − |
| 296 | − | + |
| 297 | + | + |
| 298 | + | + |
| 299 | − | − |
| 300 | − | + |
| 301 | − | + |
| 302 | − | − |
| 303 | − | − |
| 304 | − | + |
| 305 | − | − |
| 306 | − | + |
| 307 | + | − |
| 308 | − | − |
| 309 | − | − |
| 310 | − | + |
| 311 | − | − |
| 312 | − | − |
| 313 | + | + |
| 314 | + | + |
| 315 | + | + |
| 316 | + | + |
| 317 | + | − |
| 318 | − | + |
| 319 | − | + |
| 320 | − | + |
| 321 | + | − |
| 322 | + | − |
| 323 | + | − |
| 324 | − | − |
| 325 | + | − |
| 326 | + | − |
| 327 | + | − |
| 328 | + | + |
| 329 | NT | + |
| 330 | NT | + |
| 331 | NT | + |
| 332 | NT | + |
| 333 | NT | + |
| 334 | NT | − |
| 335 | NT | + |
| 336 | NT | + |
| 337 | NT | + |
| 338 | NT | + |
| 339 | NT | + |
| 340 | NT | + |
| 341 | NT | + |
| 342 | NT | + |
| 343 | NT | + |
| 344 | NT | + |
| 345 | NT | + |
| 346 | NT | + |
| 347 | NT | + |
| 348 | NT | + |
| 349 | NT | − |
| 350 | NT | + |
| 351 | NT | + |
| 352 | NT | − |
| 353 | NT | + |
| 354 | NT | + |
| 355 | NT | + |
| 356 | NT | − |
| 357 | NT | + |
| 358 | NT | + |
| 359 | NT | − |
| 360 | NT | + |
| 361 | NT | + |
| 362 | NT | + |
| 363 | NT | + |
| 364 | NT | + |
| 365 | NT | + |
| 366 | NT | + |
| 367 | NT | + |
| 368 | NT | − |
| 369 | NT | + |
| 370 | NT | − |
| 371 | NT | − |
| 372 | NT | + |
| 373 | NT | + |
| 374 | NT | + |
| 375 | NT | + |
| 376 | NT | + |
| 377 | NT | + |
| 378 | NT | + |
| 379 | NT | + |
| 380 | NT | + |
| 381 | NT | + |
| 382 | NT | + |
| 383 | NT | + |
| 384 | NT | + |
| 385 | NT | + |
| 386 | NT | + |
| 387 | NT | + |
| 388 | NT | − |
| 389 | NT | + |
| 390 | NT | + |
| 391 | NT | + |
| 392 | NT | − |
| 393 | NT | + |
| 394 | NT | − |
| 395 | NT | − |
| 396 | NT | + |
| 397 | NT | + |
| 398 | NT | − |
| 399 | NT | + |
| 400 | NT | + |
| 401 | NT | + |
| 402 | NT | + |
| 403 | NT | + |
| 404 | NT | + |
| 405 | NT | + |
| 406 | NT | + |
| 407 | NT | + |
| 408 | NT | + |
| 409 | NT | + |
| 410 | NT | + |
| 411 | NT | − |
| 412 | NT | + |
| 413 | NT | + |
| 414 | NT | + |
| 415 | NT | + |
| 416 | NT | + |
| 417 | NT | + |
| 418 | NT | − |
| 419 | NT | + |
| 420 | NT | + |
| 421 | NT | + |
| 422 | NT | + |
| 423 | NT | + |
| 424 | NT | + |
| 425 | NT | + |
| 426 | NT | + |
| 427 | NT | + |
| 428 | NT | + |
| 429 | NT | + |
| 430 | NT | + |
| 431 | NT | − |
| 432 | NT | + |
| 433 | NT | + |
| 434 | NT | + |
| 435 | NT | + |
| 436 | NT | − |
| 437 | NT | + |
| 438 | NT | + |
| 439 | NT | + |
| 440 | NT | + |
| 441 | NT | NA |
| 442 | NT | + |
| 443 | NT | + |
| 444 | NT | − |
| 445 | NT | + |
| 446 | NT | − |
| 447 | NT | − |
| 448 | NT | + |
| 449 | NT | + |
| 450 | NT | − |
| 451 | NT | + |
| 452 | NT | + |
| 453 | NT | − |
| 454 | NT | + |
| 455 | NT | + |
| 456 | NT | + |
| 457 | NT | − |
| 458 | NT | + |
| 459 | NT | + |
| 460 | NT | + |
| 461 | NT | + |
| 462 | NT | + |
| 463 | NT | + |
| 464 | NT | + |
| 465 | NT | + |
| 466 | NT | + |
| 467 | NT | + |
| 468 | NT | + |
| 469 | NT | + |
| 470 | NT | + |
| 471 | NT | + |
| 472 | NT | + |
| 473 | NT | + |
| 474 | NT | + |
| 475 | NT | + |
| 476 | NT | + |
| 477 | NT | + |
| 478 | NT | + |
| 479 | NT | + |
| 480 | NT | + |
| 481 | NT | + |
| 482 | NT | + |
| 483 | NT | − |
| 484 | NT | + |
| 485 | NT | + |
| 486 | NT | + |
| 487 | NT | − |
| 488 | NT | + |
| 489 | NT | + |
| 490 | NT | + |
| 491 | NT | + |
| 492 | NT | + |
| 493 | NT | + |
| 494 | NT | − |
| 495 | NT | − |
| 496 | NT | + |
| 497 | NT | − |
| 498 | NT | + |
| 499 | NT | + |
| 500 | NT | + |
| 501 | NT | − |
| 502 | NT | − |
| 503 | NT | + |
| 504 | NT | − |
| 505 | NT | + |
| 506 | NT | + |
| 507 | NT | + |
| 508 | NT | + |
| 509 | NT | − |
| 510 | NT | + |
| 511 | NT | − |
| 512 | NT | + |
| 513 | NT | + |
| 514 | NT | − |
| 515 | NT | − |
| 516 | NT | + |
| 517 | NT | + |
| 518 | NT | + |
| 519 | NT | − |
| 520 | NT | − |
| 521 | NT | − |
| 522 | NT | + |
| 523 | NT | + |
| 524 | NT | + |
| 525 | NT | − |
| 526 | NT | + |
| 527 | NT | − |
| 528 | NT | + |
| 529 | NT | + |
| 530 | NT | − |
| 531 | NT | + |
| 532 | NT | − |
| 533 | NT | + |
| 534 | NT | − |
| 535 | NT | − |
| 536 | NT | + |
| 537 | NT | − |
| 538 | NT | − |
| 539 | NT | − |
| 540 | NT | + |
| 541 | NT | − |
| 542 | NT | − |
| 543 | NT | − |
| 544 | NT | + |
| 545 | NT | − |
| 546 | NT | − |
| 547 | NT | + |
| 548 | NT | − |
| 549 | NT | − |
| 550 | NT | − |
| 551 | NT | + |
| 552 | NT | + |
| 553 | NT | − |
| 554 | NT | + |
| 555 | NT | − |
| 556 | NT | − |
| 557 | NT | − |
| 558 | NT | − |
| 559 | NT | − |
| 560 | NT | + |
| 561 | NT | − |
| 562 | NT | + |
| 563 | NT | + |
| 564 | NT | + |
| 565 | NT | − |
| 566 | NT | − |
| 567 | NT | − |
| 568 | NT | − |
| 569 | NT | − |
| 570 | NT | − |
| 571 | NT | − |

### In Vivo Assay

Acute IOP Response in Lasered (Hypertensive) Eyes of Conscious Cynomolgus Monkeys

Intraocular pressure (IOP) can be determined with an Alcon Pneumatonometer after light corneal anesthesia with 0.1% proparacaine. Eyes are washed with saline after each measurement. After a baseline IOP measurement, test compound is instilled in one 30 pL aliquot to the right eyes only of nine cynomolgus monkeys. Vehicle is instilled in the right eyes of six additional animals. Subsequent IOP measurements are taken at 1, 3, and 6 hours, and peak reduction in IOP is reported below in Table 2 as percent of IOP lowering versus the control for each of the given concentrations of compound. NT indicates that the compound was not tested at a given concentration.

TABLE 2

Peak % Lowering
of IOP vs.
Control

| Example No. | at 0.3% | at 1.0% |
|---|---|---|
| 26 | 9.8 | NT |
| 27 | 3.6 | NT |
| 51 | 9.5 | 18.6 |

A more detailed description of the assay used herein may be found in May et al., “Evaluation of the Ocular Hypotensive Response of Serotonin 5-HT_{1A} and 5-HT₂ Receptor Ligands in Conscious Ocular Hypertensive Cynomolgus Monkeys,” J. of Pharmacology and Experimental Therapeutics, vol. 306(1), pp. 301-309 (2003), the disclosure of which is hereby incorporated by reference as if written herein in its entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of inhibition of Rho kinase comprising contacting Rho kinase with a compound of structural Formula I 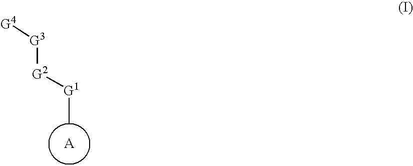
or a salt, ester, or prodrug thereof, wherein:
A is optionally substituted heteroaryl;
G¹ is optionally substituted fused bicyclic heteroaryl;
G² is selected from the group consisting of (CR^{a}R^{b})ₘZ(CR^{c}R^{d})ₚ and null;
m and p are independently 0, 1, 2, 3, or 4;
Z is selected from the group consisting of O, N(R¹), S(O)ₙ, N(R^{e})CO, CON(R^{e}), N(R^{e})SO₂, SO₂N(R^{e}), C(O), optionally substituted cycloalkyl, and null;
R^{e} is selected from the group consisting of hydrogen and optionally substituted C₁-C₄ alkyl;
n is 0, 1 or 2;
R^{a}, R^{b}, R^{c}, and R^{d} are independently selected from the group consisting of hydrogen, alkyl, amino, aminoalkyl, amidoalkyl, aminoalkylcarboxyl, carboxylalkyl, halo, heterocycloalkyl, heterocycloalkylalkyl, hydroxyalkyl, heteroarylalkyl and heterocycloalkylalkylcarboxyl;
G³ is selected from the group consisting of lower alkyl, cycloalkyl, aryl, arylalkyl, heterocycloalkyl, heteroaryl, lower alkoxy, lower alkylthio, acyl, carboxyl, sulfonamide, hydroxy and null, any of which may be optionally substituted;
G⁴ is selected from the group consisting of hydrogen, halogen, alkyl, alkoxy, amino, aminoalkyl, amido, amidoalkyl, alkylamido, aminoalkylcarboxyl, carboxyl, alkylcarboxyl, cycloalkyl, heterocycloalkyl, heterocycloalkylcarbonyl, heterocycloalkylalkyl, heterocycloalkylalkoxy, heterocycloalkylalkylcarboxy, heterocycloalkylalkylamido, aryl, arylalkoxy, arylamido, arylalkyl, arylacyl, arylcarboxy, heteroarylalkyl, and urea, any of which may be optionally substituted; and
R¹ is selected from the group consisting of alkyl, alkylcarbonyl, alkylene, alkynyl, amino, alkylamino, carbonyl, cycloalkyl, ester, heterocycloalkyl, heterocycloalkylalkyl, heteroalkyl, and hydrogen, any of which may be optionally substituted.

2. A method of inhibition of Rho kinase comprising contacting Rho kinase with a compound selected from the group consisting of Examples 1 to 571.

3. A method of treatment of a Rho kinase-mediated disease, in a patient in need of such treatment, comprising the administration of a therapeutically effective amount of a compound of structural Formula I 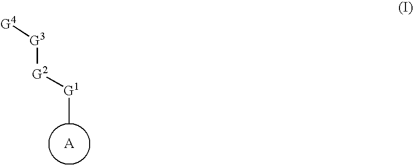
or a salt, ester, or prodrug thereof, wherein:
A is optionally substituted heteroaryl;
G¹ is optionally substituted fused bicyclic heteroaryl;
G² is selected from the group consisting of (CR^{a}R^{b})ₘZ(CR^{c}R^{d})ₚ and null;
m and p are independently 0, 1, 2, 3, or 4;
Z is selected from the group consisting of O, N(R¹), S(O)ₙ, N(R^{e})CO, CON(R^{e}), N(R^{e})SO₂, SO₂N(R^{e}), C(O), optionally substituted cycloalkyl, and null;
R^{e} is selected from the group consisting of hydrogen and optionally substituted C₁-C₄ alkyl;
n is 0, 1 or 2;
R^{a}, R^{b}, R^{c}, and R^{d} are independently selected from the group consisting of hydrogen, alkyl, amino, aminoalkyl, amidoalkyl, aminoalkylcarboxyl, carboxylalkyl, halo, heterocycloalkyl, heterocycloalkylalkyl, hydroxyalkyl, heteroarylalkyl and heterocycloalkylalkylcarboxyl;
G³ is selected from the group consisting of lower alkyl, cycloalkyl, aryl, arylalkyl, heterocycloalkyl, heteroaryl, lower alkoxy, lower alkylthio, acyl, carboxyl, sulfonamide, hydroxy and null, any of which may be optionally substituted;
G⁴ is selected from the group consisting of hydrogen, halogen, alkyl, alkoxy, amino, aminoalkyl, amido, amidoalkyl, alkylamido, aminoalkylcarboxyl, carboxyl, alkylcarboxyl, cycloalkyl, heterocycloalkyl, heterocycloalkylcarbonyl, heterocycloalkylalkyl, heterocycloalkylalkoxy, heterocycloalkylalkylcarboxy, heterocycloalkylalkylamido, aryl, arylalkoxy, arylamido, arylalkyl, arylacyl, arylcarboxy, heteroarylalkyl, and urea, any of which may be optionally substituted; and
R¹ is selected from the group consisting of alkyl, alkylcarbonyl, alkylene, alkynyl, amino, alkylamino, carbonyl, cycloalkyl, ester, heterocycloalkyl, heterocycloalkylalkyl, heteroalkyl, and hydrogen, any of which may be optionally substituted.

4. The method as recited in claim 3 wherein said Rho kinase-mediated disease is selected from the group consisting of angina, coronary artery vasospasm, myocardial infarction, coronary ischemia, congestive heart failure, cardiac allograft vasculopathy, vein graft disease and vascular restenosis, ischemic reperfusion injury, transplant reperfusion injury, cerebral artery vasospasm, stroke, cerebral ischemia, essential hypertension, pulmonary hypertension, renal hypertension, a secondary hypertensive disorder, atherosclerosis, bronchial asthma, an acute or chronic obstructive pulmonary disease, an acute or chronic pulmonary inflammatory disease, erectile dysfunction, a neurodegenerative disorder, Alzheimer's disease, multiple sclerosis, brain or spinal cord injury, a disease or trauma-related neuropathy, neuropathic pain, an autoimmune disease, a chronic musculoskeletal inflammatory disease, rheumatoid arthritis, osteoarthritis, a chronic inflammatory bowel disease, Crohn's disease, ulcerative colitis, acute or chronic inflammatory pain, osteoporosis, a bone disorder, cancer, a disease of pathological angiogenesis, and an ophthalmic disease.

5. The method as recited in claim 4, wherein said Rho kinase-mediated disease is an ophthalmic disease.

6. The method as recited in claim 5, wherein said ophthalmic disease is selected from the group consisting of elevated intraocular pressure and glaucoma.

7. A method of treatment of a Rho kinase-mediated disease, in a patient in need of such treatment, comprising the administration of a therapeutically effective amount of a compound selected from the group consisting of Examples 1 to 571.

8. A method of treatment of a Rho kinase-mediated disease comprising the administration of
a. a therapeutically effective amount of a compound of structural Formula I 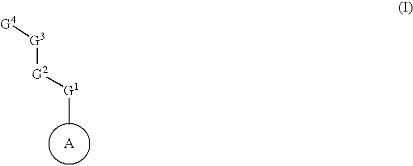
or a salt, ester, or prodrug thereof, wherein:
A is optionally substituted heteroaryl;
G¹ is optionally substituted fused bicyclic heteroaryl;
G² is selected from the group consisting of (CR^{a}R^{b})ₘZ(CR^{c}R^{d})ₚ and null;
m and p are independently 0, 1, 2, 3, or 4;
Z is selected from the group consisting of O, N(R¹), S(O)ₙ, N(R^{e})CO, CON(R^{e}), N(R^{e})SO₂, SO₂N(R^{e}), C(O), optionally substituted cycloalkyl, and null;
R^{e} is selected from the group consisting of hydrogen and optionally substituted C₁-C₄ alkyl;
n is 0, 1 or 2;
R^{a}, R^{b}, R^{c}, and R^{d} are independently selected from the group consisting of hydrogen, alkyl, amino, aminoalkyl, amidoalkyl, aminoalkylcarboxyl, carboxylalkyl, halo, heterocycloalkyl, heterocycloalkylalkyl, hydroxyalkyl, heteroarylalkyl and heterocycloalkylalkylcarboxyl;
G³ is selected from the group consisting of lower alkyl, cycloalkyl, aryl, arylalkyl, heterocycloalkyl, heteroaryl, lower alkoxy, lower alkylthio, acyl, carboxyl, sulfonamide, hydroxy, and null, any of which may be optionally substituted;
G⁴ is selected from the group consisting of hydrogen, halogen, alkyl, alkoxy, amino, aminoalkyl, amido, amidoalkyl, alkylamido, aminoalkylcarboxyl, carboxyl, alkylcarboxyl, cycloalkyl, heterocycloalkyl, heterocycloalkylcarbonyl, heterocycloalkylalkyl, heterocycloalkylalkoxy, heterocycloalkylalkylcarboxy, heterocycloalkylalkylamido, aryl, arylalkoxy, arylamido, arylalkyl, arylacyl, arylcarboxy, heteroarylalkyl, and urea, any of which may be optionally substituted; and
R¹ is selected from the group consisting of alkyl, alkylcarbonyl, alkylene, alkynyl, amino, alkylamino, carbonyl, cycloalkyl, ester, heterocycloalkyl, heterocycloalkylalkyl, heteroalkyl, and hydrogen, any of which may be optionally substituted; and
b. another therapeutic agent.

9. A method for:
a. reducing apoptosis of human embryonic stem cells;
b. increasing survival of human embryonic stem cells;
c. increasing cloning efficiency of human embryonic stem cells after gene transfer; and
d. enhancing differentiation of cultured human embryonic stem cells any one of said methods comprising the contacting of at least one human embryonic stem cell with an effective amount of a compound of structural Formula I 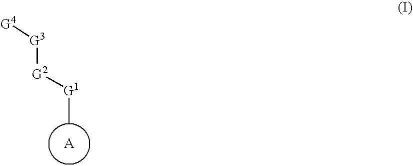
or a salt, ester, or prodrug thereof, wherein:
A is optionally substituted heteroaryl;
G¹ is optionally substituted fused bicyclic heteroaryl;
G² is selected from the group consisting of (CR^{a}R^{b})ₘZ(CR^{c}R^{d})ₚ and null;
m and p are independently 0, 1, 2, 3, or 4;
Z is selected from the group consisting of O, N(R¹), S(O)ₙ, N(R^{e})CO, CON(R^{e}), N(R^{e})SO₂, SO₂N(R^{e}), C(O), optionally substituted cycloalkyl, and null;
R^{e} is selected from the group consisting of hydrogen and optionally substituted C₁-C₄ alkyl;
n is 0, 1 or 2;
R^{a}, R^{b}, R^{c}, and R^{d} are independently selected from the group consisting of hydrogen, alkyl, amino, aminoalkyl, amidoalkyl, aminoalkylcarboxyl, carboxylalkyl, halo, heterocycloalkyl, heterocycloalkylalkyl, hydroxyalkyl, heteroarylalkyl and heterocycloalkylalkylcarboxyl;
G³ is selected from the group consisting of lower alkyl, cycloalkyl, aryl, arylalkyl, heterocycloalkyl, heteroaryl, lower alkoxy, lower alkylthio, acyl, carboxyl, sulfonamide, hydroxy, and null, any of which may be optionally substituted;
G⁴ is selected from the group consisting of hydrogen, halogen, alkyl, alkoxy, amino, aminoalkyl, amido, amidoalkyl, alkylamido, aminoalkylcarboxyl, carboxyl, alkylcarboxyl, cycloalkyl, heterocycloalkyl, heterocycloalkylcarbonyl, heterocycloalkylalkyl, heterocycloalkylalkoxy, heterocycloalkylalkylcarboxy, heterocycloalkylalkylamido, aryl, arylalkoxy, arylamido, arylalkyl, arylacyl, arylcarboxy, heteroarylalkyl, and urea, any of which may be optionally substituted; and
R¹ is selected from the group consisting of alkyl, alkylcarbonyl, alkylene, alkynyl, amino, alkylamino, carbonyl, cycloalkyl, ester, heterocycloalkyl, heterocycloalkylalkyl, heteroalkyl, and hydrogen, any of which may be optionally substituted.

10. A compound of structural Formula I: 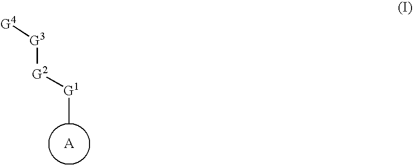
or a salt, ester, or prodrug thereof, wherein:
A is optionally substituted heteroaryl;
G¹ is optionally substituted fused bicyclic heteroaryl;
G² is selected from the group consisting of (CR^{a}R^{b})ₘZ(CR^{c}R^{d})ₚ and null;
m and p are independently 0, 1, 2, 3, or 4;
Z is selected from the group consisting of O, N(R¹), S(O)ₙ, N(R^{e})CO, CON(R^{e}), N(R^{e})SO₂, SO₂N(R^{e}), C(O), optionally substituted cycloalkyl, and null;
R^{e} is selected from the group consisting of hydrogen and optionally substituted C₁-C₄ alkyl;
n is 0, 1 or 2;
R^{a}, R^{b}, R^{c}, and R^{d} are independently selected from the group consisting of hydrogen, alkyl, amino, aminoalkyl, amidoalkyl, aminoalkylcarboxyl, carboxylalkyl, halo, heterocycloalkyl, heterocycloalkylalkyl, hydroxyalkyl, heteroarylalkyl and heterocycloalkylalkylcarboxyl;
G³ is selected from the group consisting of lower alkyl, cycloalkyl, aryl, arylalkyl, heterocycloalkyl, heteroaryl, lower alkoxy, lower alkylthio, acyl, carboxyl, sulfonamide, hydroxy, and null, any of which may be optionally substituted;
G⁴ is selected from the group consisting of hydrogen, halogen, alkyl, alkoxy, amino, aminoalkyl, amido, amidoalkyl, alkylamido, aminoalkylcarboxyl, carboxyl, alkylcarboxyl, cycloalkyl, heterocycloalkyl, heterocycloalkylcarbonyl, heterocycloalkylalkyl, heterocycloalkylalkoxy, heterocycloalkylalkylcarboxy, heterocycloalkylalkylamido, aryl, arylalkoxy, arylamido, arylalkyl, arylacyl, arylcarboxy, heteroarylalkyl, and urea, any of which may be optionally substituted; and
R¹ is selected from the group consisting of alkyl, alkylcarbonyl, alkylene, alkynyl, amino, alkylamino, carbonyl, cycloalkyl, ester, heterocycloalkyl, heterocycloalkylalkyl, heteroalkyl, and hydrogen, any of which may be optionally substituted.

11. The compound as recited in claim 10, or a salt, ester, or prodrug thereof, wherein:
A is selected from the group consisting of optionally substituted monocyclic 5 to 6 membered heteroaryl containing at least one ring nitrogen, or an optionally substituted bicyclic heteroaryl which comprises a five-membered ring fused to a six-membered ring and which contains at least one ring nitrogen.

12. The compound as recited in claim 11, or a salt, ester, or prodrug thereof, wherein G¹ is selected from the group consisting of: 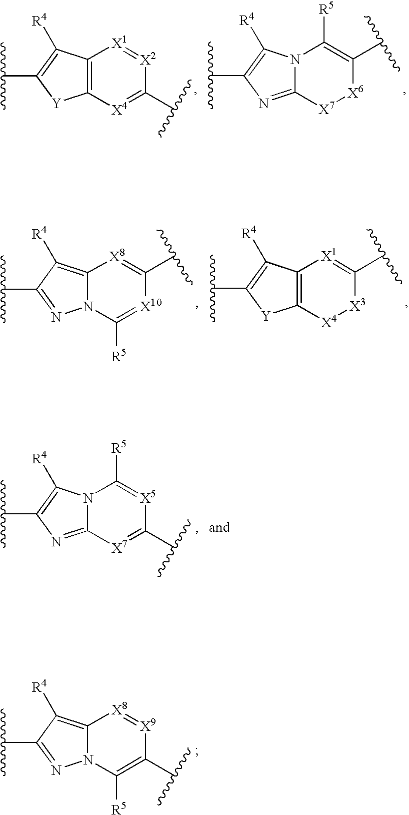
X¹ is N or C(R⁶);
X² is N or C(R⁷);
X³ is N or C(R⁸);
X⁴ is N or C(R⁹);
X⁵ is N or C(R¹⁰);
X⁶ is N or C(R¹¹);
X⁷ is N or C(R¹²);
X⁸ is N or C(R¹³);
X⁹ is N or C(R¹⁴);
X¹⁰ is N or C(R¹⁵);
Y is O or S; and
R⁴-R¹⁵ are independently selected from the group consisting of hydrogen, halogen, lower alkyl, cycloalkyl, aryl, heterocycloalkyl, heteroaryl, lower alkoxy, lower alkylthio, lower haloalkyl, acyl, amino, carboxyl, cyano, and nitro, any of which may be optionally substituted.

13. The compound as recited in claim 12, or a salt, ester, or prodrug thereof, wherein A is selected from the group consisting of 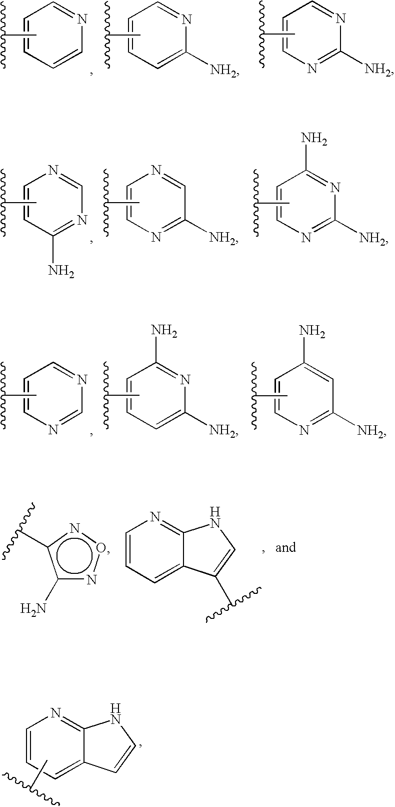
any of which may be optionally substituted.

14. The compound as recited in claim 13, or a salt, ester, or prodrug thereof, wherein
G² is (CR^{a}R^{b})ₘZ(CR^{c}R^{d})ₚ;
m and p are independently 0, 1, or 2;
Z is selected from the group consisting of O, N(R¹), S(O)ₙ, N(R^{e})CO, CON(R^{e}), C(O), and null;
R^{e} is selected from the group consisting of hydrogen and optionally substituted C₁-C₄ alkyl; and
n is 0 or 2.

15. The compound as recited in claim 14, or a salt, ester, or prodrug thereof, wherein G¹ is:

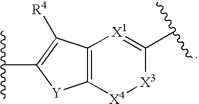

16. The compound as recited in claim 15, or a salt, ester, or prodrug thereof, wherein A is selected from the group consisting of

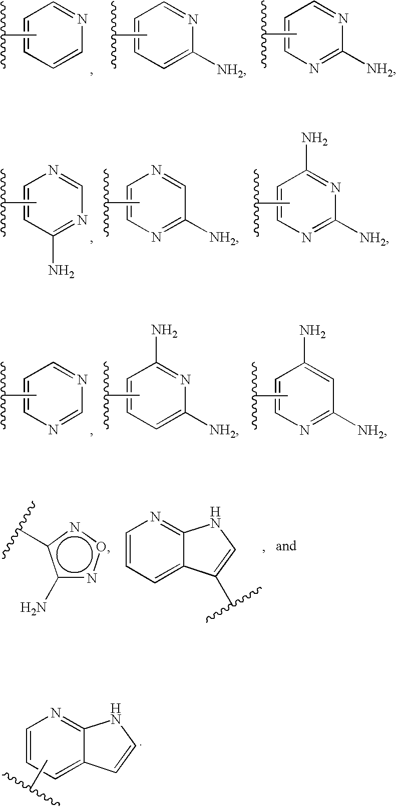

17. The compound as recited in claim 16, or a salt, ester, or prodrug thereof, having structural Formula II 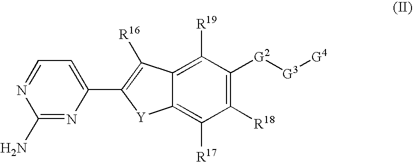
or a salt, ester, or prodrug thereof, wherein:
Y is O or S;
G² is (CR^{a}R^{b})ₘZ(CR^{c}R^{d})ₚ;
m and p are independently 0, 1, or 2;
Z is selected from the group consisting of O, N(R¹), S(O)ₙ, N(R^{e})CO, CON(R^{e}), C(O), and null;
R^{e} is selected from the group consisting of hydrogen and optionally substituted C₁-C₄ alkyl; and
n is 0 or 2;
G³ is selected from the group consisting of lower alkyl, cycloalkyl, aryl, arylalkyl, heterocycloalkyl, heteroaryl, lower alkoxy, lower alkylthio, acyl, carboxyl, sulfonamide, hydroxy, and null, any of which may be optionally substituted;
G⁴ is selected from the group consisting of hydrogen, halogen, alkyl, alkoxy, amino, aminoalkyl, amido, amidoalkyl, alkylamido, aminoalkylcarboxyl, carboxyl, alkylcarboxyl, cycloalkyl, heterocycloalkyl, heterocycloalkylcarbonyl, heterocycloalkylalkyl, heterocycloalkylalkoxy, heterocycloalkylalkylcarboxy, heterocycloalkylalkylamido, aryl, arylalkoxy, arylamido, arylalkyl, arylacyl, arylcarboxy, heteroarylalkyl, and urea, any of which may be optionally substituted;
R¹⁶ is selected from the group consisting of lower alkenyl, alkynyl, lower alkyl, alkylthio, haloalkyl, heteroalkyl, hydroxyalkyl, halogen, and hydrogen; and
R¹⁷-R¹⁹ are independently selected from the group consisting of acyl, lower alkenyl, alkynyl, lower alkoxy, lower alkoxyalkyl, lower alkyl, alkylthio, amido, amino, aminoalkyl, aminocarbonyl, carboxyl, haloalkyl, hydroxyalkyl and hydrogen, any of which may be optionally substituted.

18. The compound as recited in claim 17, or a salt, ester, or prodrug thereof, wherein:
Y is S;
R¹⁶ is selected from the group consisting of lower alkyl and hydrogen; and
R¹⁷-R¹⁹ are all hydrogen.

19. The compound as recited in claim 18, or a salt, ester, or prodrug thereof, wherein G³ is selected from the group consisting of aryl, heterocycloalkyl, heteroaryl, any of which may be optionally substituted.

20. The compound as recited in claim 19, or a salt, ester, or prodrug thereof, wherein either
m and p are both 0; and
Z is selected from the group consisting of O, NH, S, and C(O); or
m is 1;
Z is null; and
p is 0.

21. The compound as recited in claim 20, or a salt, ester, or prodrug thereof, wherein R¹⁶ is selected from the group consisting of methyl, ethyl, heteroalkyl, and halogen.

22. The compound as recited in claim 21, or a salt, ester, or prodrug thereof, wherein G⁴ is selected from the group consisting of hydrogen, halogen, alkoxy, amino, alkylamido, carboxyl, alkylcarboxyl, heterocycloalkylalkyl, heterocycloalkylalkoxy, heterocycloalkylalkylcarboxy, and heterocycloalkylalkylamido, any of which may be optionally substituted.

23. A compound selected from the group consisting of Examples 3-93 and 95-571.

24. A compound as recited in claim 10 for use as a medicament.

25. A compound as recited in claim 10 for use in the manufacture of a medicament for the prevention or treatment of a disease or condition ameliorated by the inhibition of Rho kinase.

26. A pharmaceutical composition comprising a compound as recited in claim 10 together with a pharmaceutically acceptable carrier.
